(19) United States
(12) Patent Application Publication
(10) Pub. No.: US 2025/0222102 A1
(43) Pub. Date: Jul. 10, 2025

(54) NK CELLS EXPRESSING IL-15 AND CHECKPOINT INHIBITORS FOR THE TREATMENT OF CANCER

(71) Applicant: City of Hope, Duarte, CA (US)

(72) Inventors: Jianhua Yu, Duarte, CA (US); Michael A. Caligiuri, Duarte, CA (US)

(21) Appl. No.: 18/685,854

(22) PCT Filed: Aug. 24, 2022
(86) PCT No.: PCT/US2022/075428

§ 371 Date: Feb. 22, 2024

Related U.S. Application Data
(60) Provisional application No. 63/236,627, filed on Aug. 24, 2021.

(51) Int. Cl. A61K 40/15; A61K 40/35; A61K 40/42; A61P 35/00; C07K 14/54

(52) U.S. Cl. CPC A61K 40/15; A61K 40/35; A61K 40/4204; A61K 40/421; A61K 40/4234; A61P 35/00; C07K 14/5443; A61K 2239/31; A61K 2239/38

(57) ABSTRACT

Provided herein, inter alia, are PD-L1(+) natural killer cells that express soluble IL-15, PD-L1(+) natural killer cells that express soluble IL-15 and truncated EGFR, and methods of treating cancer using the PD-L1(+) natural killer cells. In an aspect is provided a method of treating cancer in a patient in need thereof comprising administering to the patient an effective amount of PD-L1(+) natural killer cells that express soluble IL-15.

### CROSS-REFERENCE TO RELATED APPLICATIONS

This application claims the benefit of priority to U.S. Application No. 63/236,627 filed Aug. 24, 2021, the disclosure of which is incorporated by reference herein in its entirety.

### SEQUENCE LISTING

The material in the accompanying Sequence Listing is hereby incorporated by reference in its entirety. The accompanying file, named “048440-820001WO_SL_ST26.xml” was created on Aug. 24, 2022 and is 7,248 bytes. The file can be accessed using Microsoft Word on a computer that uses Windows OS.

### BACKGROUND

Inhibition of the programmed death-1/programmed death ligand-1 (PD-1/PD-L1) pathway has become a very powerful therapeutic strategy for patients with cancer, and has shown unprecedented clinical responses in advanced liquid and solid tumors. PD-1 monoclonal antibodies (mAbs) pembrolizumab (KEYTRUDA®) and nivolumab (OPDIVO®) are FDA-approved to treat melanoma, kidney cancer, head and neck cancers, and Hodgkin's lymphoma. Three PD-L1 mAbs, atezolizumab (TECENTRIQ®), avelumab (BAVENCIO®), and durvalumab (IMFINZI®), are FDA-approved to treat non-small cell lung cancer (NSCLC), bladder cancer, and Merkel cell carcinoma of the skin. However, the overall response rate to anti-PD-L1 therapy is still very low in patients with melanoma (26%), NSCLC (21%), and renal cell carcinoma (13%). In addition, anti-PD-L1 therapy can also show an unexplained clinical response in the absence of PD-L1 expression on the tumor cells.

Tumor cells in the tumor microenvironment (TME) can upregulate PD-L1 after encountering activated T cells via their secretion of interferon gamma (IFN-γ). Upon binding to PD-1, PD-L1 delivers a suppressive signal to T cells and an anti-apoptotic signal to tumor cells, leading to T cell dysfunction and tumor survival. Therefore, anti-PD-1/PD-L1 therapy aims to remove this immune suppression and activate the T cell response against cancer. It has been reported that PD-L1 is not only expressed on tumor cells but is also found on immune cells such as T cells, natural killer (NK) cells and macrophages within the TME. However, the function and the mechanism of action of PD-L1 on NK cells remain unexplored. It is also unknown as to whether and how anti-PD-L1 mAbs can modulate the function of these NK cells expressing PD-L1. Unraveling these mechanisms will likely play an important role in the clinical effectiveness of anti-PD-1/PD-L1 therapy.

NK cells comprise a group of innate cytolytic effector cells that participate in immune surveillance against cancer and viral infection. NK cells become cytolytic without prior activation especially when they encounter cells lacking self-MHC Class I molecules. Downregulation of MHC occurs in the setting of cancer, allowing NK cells to recognize and lyse malignant cells. Activated NK cells exert strong cytotoxic effects via multiple mechanisms involving perform, granzyme B, TRAIL, or FASL. NK cells also produce IFN-γ, which not only directly affects target cells, but also activates macrophages and T cells to kill tumor cells or enhance the antitumor activity of other immune cells.

Provided herein are, inter alia, solutions to these and other problems in the art.

### BRIEF SUMMARY

In an aspect is provided PD-L1(+) natural killer cells that express soluble IL-15. In embodiments, the PD-L1(+) natural killer cells express soluble IL-15 and truncated EGFR.

In an aspect is provided a method of treating cancer in a patient in need thereof comprising administering to the patient an effective amount of PD-L1(+) natural killer cells that express soluble IL-15. In embodiments, the PD-L1(+) natural killer cells express soluble IL-15 and truncated EGFR. The method optionally further includes administering to the patient an effective amount of a checkpoint inhibitor, such as a PD-1 inhibitor or a PD-L1 inhibitor.

In an aspect is provided a nucleic acid encoding a soluble IL-15 protein, wherein the soluble IL-15 protein includes an IL-2 signal peptide and an IL-15 protein.

In an aspect is provided a recombinant soluble IL-15 protein, wherein the soluble IL-15 protein includes an IL-2 signal peptide and an IL-15 protein.

These and other aspects and embodiments are described in detail herein.

### BRIEF DESCRIPTION OF THE DRAWINGS

FIGS. 1A-1C show expression of truncated (t) EGFR, soluble IL-15 (sIL15), and PD-L1 on activated expanded transduced cord blood natural killer (CB-NK) cells at day of harvest (Day 17). Transduction efficiency as assessed by flow cytometry of the CB-NK cells with the vector containing only tEGFR (FIG. 1A) or a vector containing tEGFR and sIL15 (FIG. 1B). FIG. 1C shows the expression of PD-L1 on the same CB-NK cells on Day 17, having been incubated overnight in culture with IL-12 and IL-18.

FIG. 2 shows the cytotoxicity of CB-NK cells expressing soluble IL-15 and PD-L1. Frozen COH06 product was tested for cytotoxicity against the human non-small cell lung cancer (NSCLC) cell line A549. Cytotoxicity was tested after co-culture of NK cells for four hours with A549 cells at different ratios and compared to cytotoxicity of CB-NK cells transduced only with tEGFR without overnight incubation in IL-12 plus IL-18.

FIG. 3 shows IL-15 production by COH06 (shown as sIL15 PD-L1, i.e., the bar to the right of each point on the x-axis). IL-15 was quantified from the supernatant of culture media at Day 17 before harvest from four samples. NT=non-transduced CB-NK cells and serve as a negative control. The x-axis shows identification numbers for four different CB donors.

FIGS. 4A-4B show quantified bio-layer interferometry (BLI) data of A549-tumor bearing-mice treated with COH06 at various doses without atezolizumab (FIG. 4A) and with atezolizumab (FIG. 4B). A549 tumor cells express a luciferase gene. Note: 5e6=5 million; 10e6=10 million; 20e6 is 20 million).

FIGS. 5A-5E show bio-layer interferometry (BLI) images of A549-tumor bearing-mice treated with COH06 at various doses with or without atezolizumab. A549 tumor cells express a luciferase gene.

FIGS. 6A-6D provide a dose-finding study using a weekly schedule of COH06 administration, as is planned in the clinical study. Following A549 non-small cell lung cancer (NSCLC) inoculation, a weekly administration of COH06 was undertaken as shown in FIG. 6A. The cohorts are shown in FIG. 6B. FIGS. 6C-6D show the anti-tumor efficacy as quantified by bioluminescence. The study demonstrated in vivo efficacy of treatment with 10 million cryopreserved COH06 cells in 2 of the 3 mice, and complete efficacy with treatment of 20 million cryopreserved, transduced, expanded umbilical cord blood NK cells from donor #18 expressing sIL-15 and PD-L1 following ex vivo activation with IL-12 and IL-18 (COH06). Histologic analysis at day 35 shows no evidence of toxicity (data not shown).

FIGS. 7A-7G show the characterization of sIL15-PDL1⁺ TRACK NK cells using the methods provided herein including embodiments thereof. FIG. 7A shows the fold change in sIL15-PDL1⁺ TRACK NK cells following expansion. FIG. 7B shows the purity of the sIL15-PDL1⁺ TRACK NK cells compared to non-transduced (NT) NK cells. FIG. 7C shows the percentage of sIL15-PDL1⁺ TRACK NK cells that expressed PD-L1 following overnight incubation with IL-12 and IL-18. FIG. 7D shows sIL15 secretion data from sIL15-PDL1⁺ TRACK NK cells compared to NT NK cells. FIG. 7E shows the vector copy number of sIL15-PDL1⁺ TRACK NK cell product. FIG. 7F shows the recovery and viability of sIL15-PDL1⁺ TRACK NK cells following cryopreservation. FIG. 7G shows quantified autonomous or dysregulated growth data of sIL15-PDL1⁺ TRACK NK cells compared to NT NK cells.

FIGS. 8A-8E show in vitro functional activity of sIL15-PDL1⁺ TRACK NK cells. FIGS. 8A-8B show the cytotoxicity of sIL15-PDL1⁺ TRACK NK cells against NSCLC cell lines using the Real-Time Cell Lysis Assay (RTCA). FIG. 8C shows tumor cell growth when A549 or H460 cells were cocultured with sIL15-PDL1⁺ TRACK NK cells. FIG. 8D shows secretion of IFN-γ, TNF-α, and granzyme B (GZMB) from sIL15-PDL1⁺ TRACK NK cells compared to NT NK cells. FIG. 8E shows expression of activation receptors (e.g. CD69, CD25, and TRAIL) and the inhibitory NKG2A receptor on sIL15-PDL1⁺ TRACK NK cells compared to NT NK cells.

FIGS. 9A-9H show in vivo functional activity of sIL15-PDL1⁺ TRACK NK cells. FIG. 9A shows the experimental timeline for treating a model of A549 NSCLC with sIL15-PDL1⁺ TRACK NK cells. FIGS. 9B-9C show quantified bio-layer interferometry (BLI) data of A549-tumor bearing-mice treated with sIL15-PDL1⁺ TRACK NK cells, sIL15 NK cells, or NT NK cells. FIG. 9D shows the experimental timeline for treating a model of A549 NSCLC with sIL15-PDL1⁺ TRACK NK cells at various doses. FIGS. 9E-9F show quantified bio-layer interferometry (BLI) data of A549-tumor bearing-mice treated with sIL15-PDL1⁺ TRACK NK cells at three different doses. FIG. 9G shows quantified data of the number of metastatic tumor nodules found in the lungs of A549-tumor bearing mice treated with sIL15-PDL1⁺ TRACK NK cells at three different doses. FIG. 9H shows quantified data for human NK cell persistence in vivo.

FIGS. 10A-10G show in vivo functional activity of sIL15-PDL1⁺ TRACK NK cells combined with atezolizumab. FIG. 10A shows the experimental timeline for treating a model of H460 NSCLC with sIL15-PDL1⁺ TRACK NK cells. FIGS. 10B-C show quantified bio-layer interferometry (BLI) data of H460-tumor bearing-mice treated with sIL15-PDL1⁺ TRACK NK cells. FIG. 10D shows the survival curve of H460-tumor bearing mice treated with sIL15-PDL1⁺ TRACK NK cells or vehicle. FIG. 10E shows the experimental timeline for treating a model of A549NSCLC with a combination of sIL15-PDL1⁺ TRACK NK cells and atezolizumab. FIGS. 10F-10G show quantified bio-layer interferometry (BLI) data of A549-tumor bearing-mice treated with sIL15-PDL1⁺ TRACK NK cells and atezolizumab.

FIGS. 11A-11G show safety evaluation data of sIL15-PDL1⁺ NK cells. FIG. 11A shows the experimental timeline for sIL15-PDL1⁺ NK cell treatment, blood sampling, and measurements of body weight and body temperature. FIG. 11B shows quantified body weight and body temperature data of animals undergoing sIL15-PDL1⁺ NK cell treatment. FIG. 11C shows quantified data of the percentage of hCD45⁺hCD56⁺ cell population in the blood of mice. FIG. 11D shows quantified data of the absolute numbers of hCD45⁺hCD56⁺ cells in 1 mL of mouse blood. FIG. 11E shows quantified data of complete blood counts (CBCs) from mice receiving sIL15-PDL1⁺ NK cell treatment. FIG. 11F shows quantified data of liver function (ALT, AST, ALP) and kidney function parameters (CK, ALB, BUN) from mice receiving sIL15-PDL1⁺ NK cell treatment. FIG. 11G shows quantified data from the cytokine profiling assessment in mice receiving sIL15-PDL1⁺ NK cell treatment.

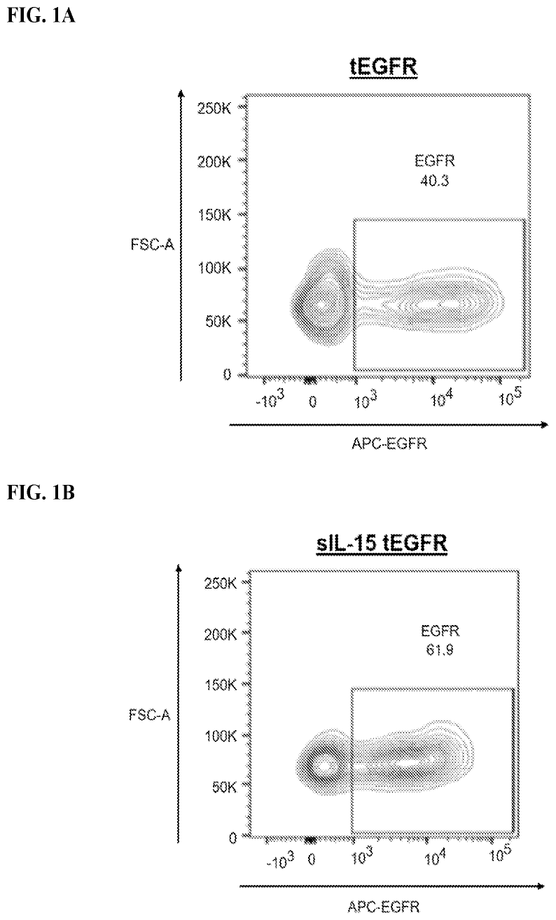

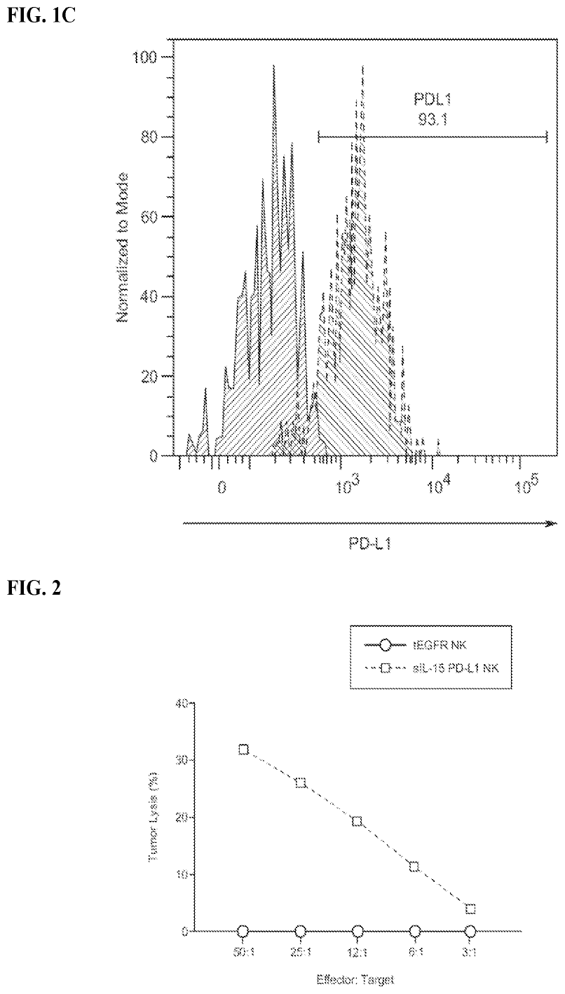

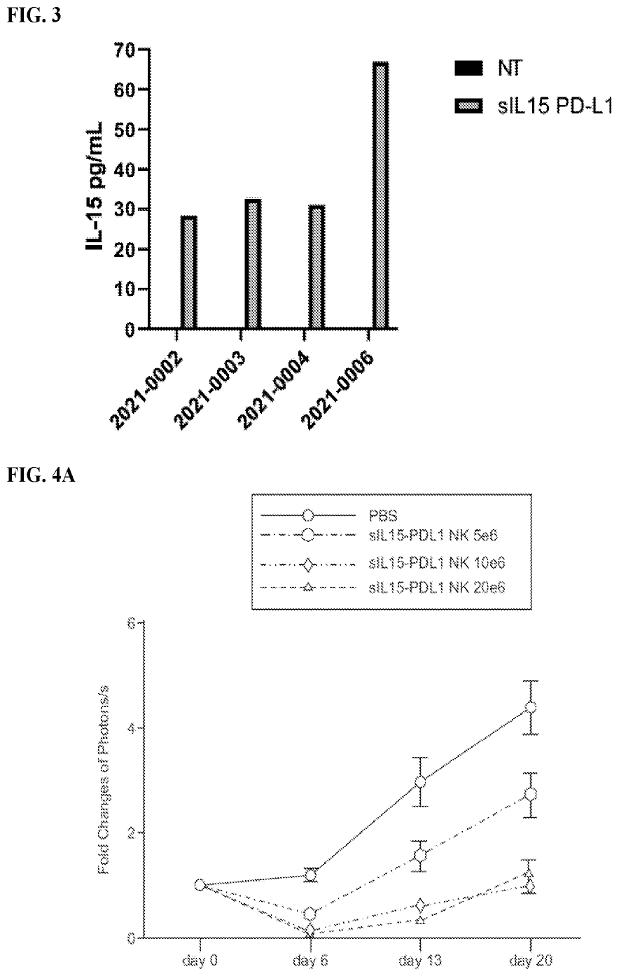

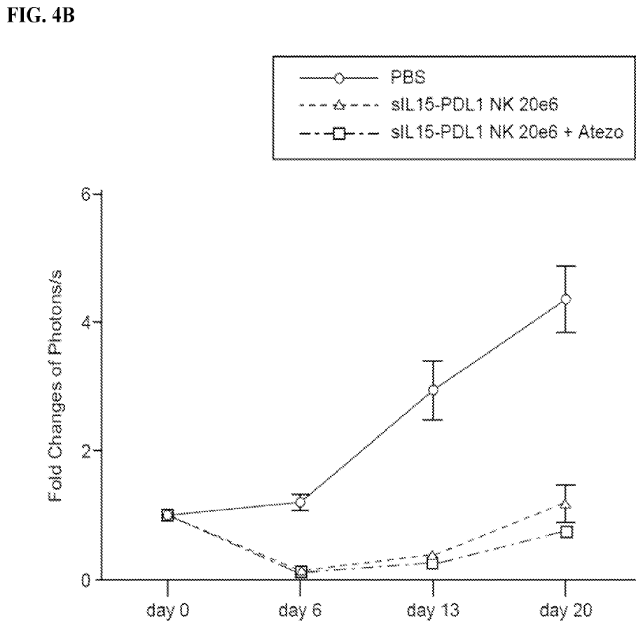

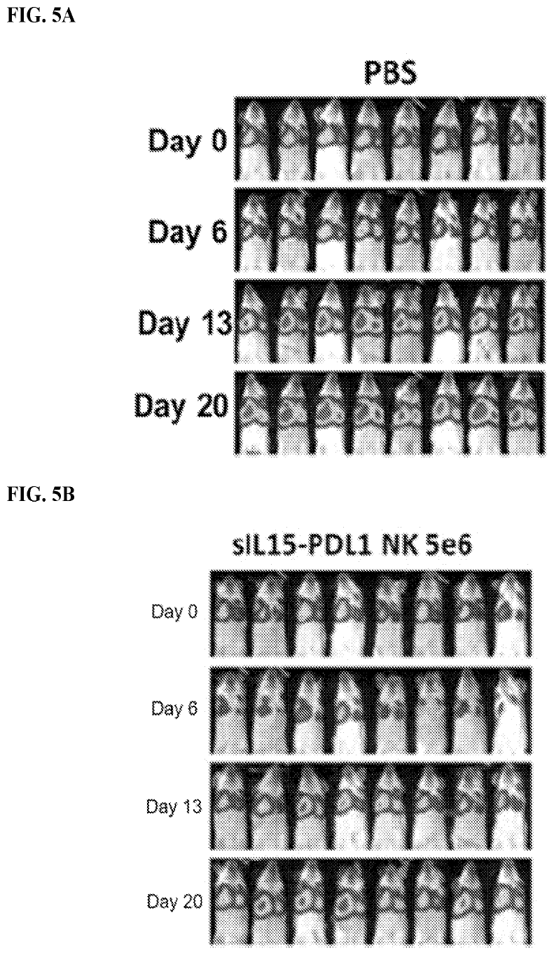

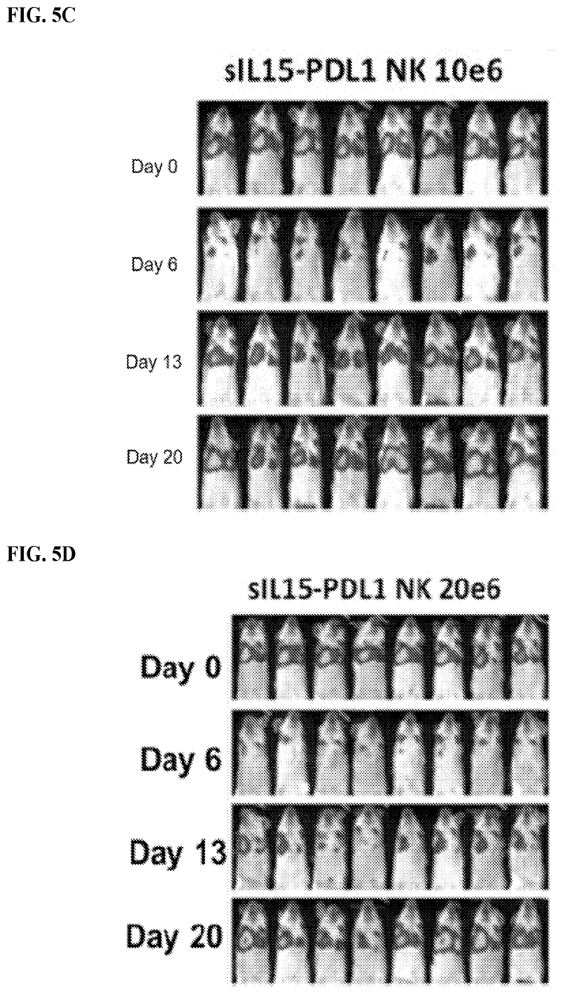

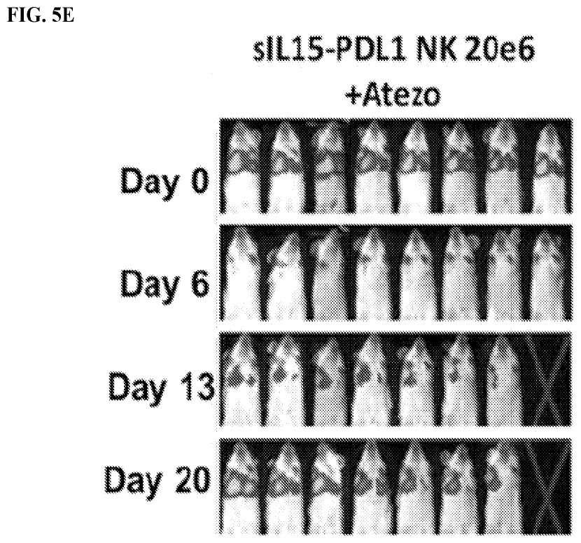

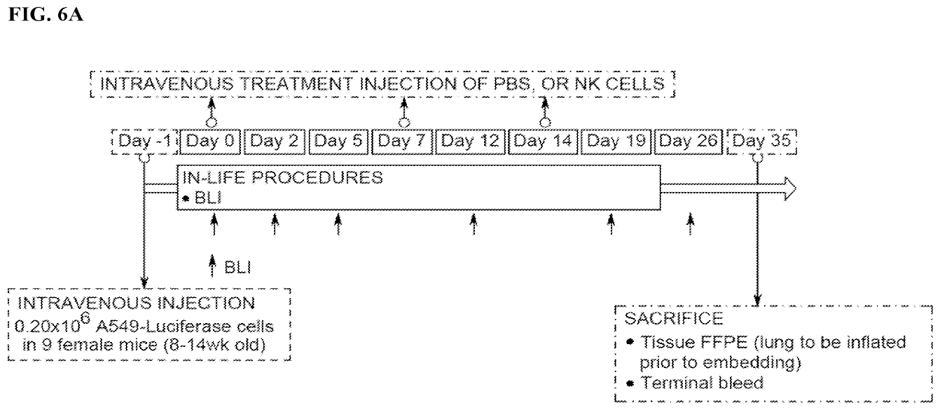

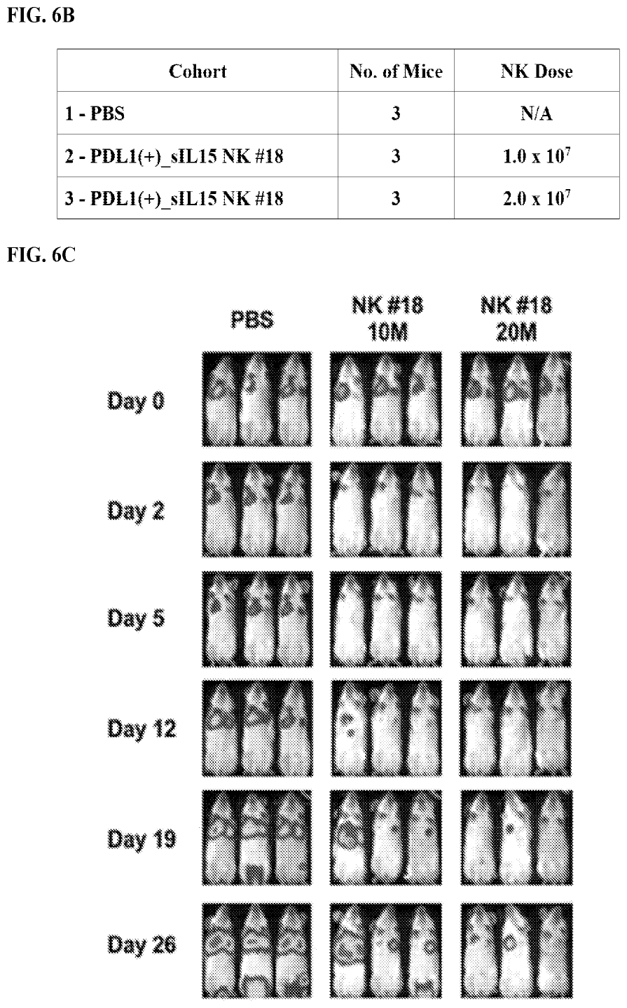

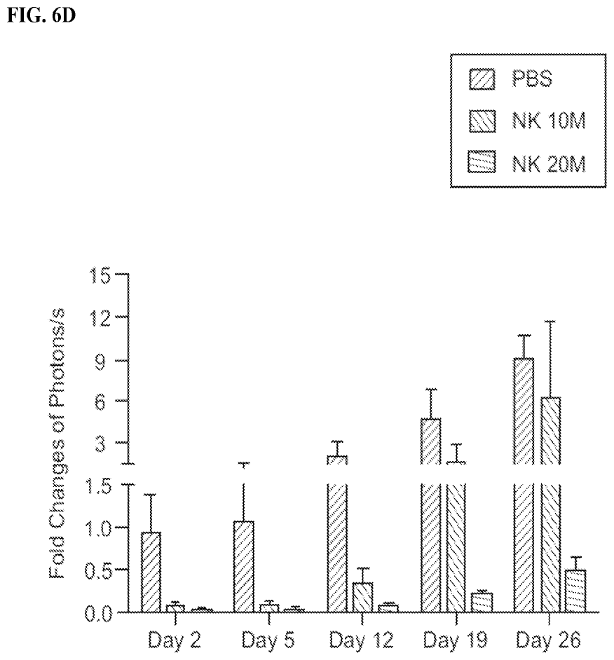

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). All publications mentioned herein are incorporated herein by reference to disclose and describe the methods and/or materials in connection with which the publications are cited.

The singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a,” “an,” “one or more,” and “at least one” can be used interchangeably.

The term “about” means a range of values including the specified value, which a person of ordinary skill in the art would consider reasonably similar to the specified value. In embodiments, “about” means within a standard deviation using measurements generally acceptable in the art. In embodiments, “about” means a range extending to +/−10% of the specified value. In embodiments, about means the specified value. When used with reference to days or weeks, “about” refers to +/−2 days or +/−1 day.

The term “recombinant” when used with reference, e.g., to a natural killer cell, indicates that the natural killer cell has been modified by the introduction of a heterologous nucleic acid or protein or the alteration of a native nucleic acid or protein, or that the cell is derived from a cell so modified. Thus, for example, recombinant natural killer cells express genes that are not found within the native (non-recombinant) form of the cell or express native genes that are otherwise abnormally expressed, under expressed or not expressed at all. Thus, for example, PD-L1(+) natural killer cells that express soluble IL-15 are recombinant PD-L1(+) natural killer cells. Similarly, the PD-L1(+) natural killer cells that express soluble IL-15 and truncated EGFR are recombinant PD-L1(+) natural killer cells.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The terms “natural killer cells” and “NK cells” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a faster immune reaction.

The term “PD-L1(+) natural killer (NK) cell” is a natural killer cell that expresses PD-L1 protein. In embodiments, the PD-L1(+) natural killer cell expresses cell surface PD-L1. In embodiments, the PD-L1(+) natural killer cell is a recombinant PD-L1(+) natural killer cell.

The term “population of PD-L1(+) natural killer (NK) cells” refers to a plurality of PD-L1(+) natural killer (NK) cells.

The terms “COH06”, “sIL15-PDL1⁺ TRACK NK cells” or “enhanced CB-NK cells” refer to activated PD-L1(+) cord blood (CB) NK cells successfully transduced with a retrovirus encoding the sIL-15 gene and the tEGFR gene. Following treatment with the retrovirus, RRV-sIL-15 tEGFR, the natural killer (NK) cells were stimulated with cytokines IL-12 and IL-18 and following expansion, cryopreserved in CryoStor® CS5 (STEMCELL®) using a controlled rate freezer. The product administered to patients also contains PD-L1(+) CB-NK cells not transduced with sIL-15 or tEGFR. We refer to these cells as activated PD-L1 (+) untransduced NK cells. Manufacture of the investigational NK cell product will be conducted under cGMP at the Center for Biomedicine and Genetics (CBG) located at the City of Hope. Cord blood donors are screened in accordance with 21 CFR 1270. The NK cells are purified from cord blood using a RosetteSep Human NK Cell Enrichment Cocktail followed by centrifugation through a Ficoll-Paque gradient to develop an umbilical cord NK cell bank. The enriched NK cells are cryopreserved in CryoStor CS5; one million cells are frozen separately to be used in the assessment of the cells' proliferation capacity (17 days expansion at small scale in the presence of IL-2 and irradiated K562 feeders). NK cells that have demonstrated sufficient expansion capacity from the 1×10⁶ aliquot such that the remaining cells would be capable of generating at least 2×10⁹ cells post full-scale expansion are subsequently thawed and will be used for cell therapy productions. The NK cells will then be co-cultured with the irradiated K562 feeder cells expressing membrane-bound (IL-21) and CD-137L and exogenous IL-2. On day 7, expanded NK cells are transduced with the retroviral vector (RRV_sIL-15_tEGFR) carrying the human IL-15 gene and the truncated EGFR. Following transduction, the cells are further expanded with additional irradiated K562 feeder cells. On day 16, the cytokines IL-18 and IL-12 are added to the cell culture to harvest to upregulate endogenous expression of PD-L1 on the sIL15+ NK cells. On day 17, the cells are harvested and cryopreserved. The Release Testing for product COH06 is shown in Table 1. PD-L1(+) natural killer cells are described in WO 2020/264043, the disclosure of which is incorporated by reference herein in its entirety.

TABLE 1

| Assay | Testing Lab | Specification |
|---|---|---|
| Sterility USP | BioReliance | No Growth |
| Mycolasma USP | BioReliance | Negative |
| Endotoxin | BioReliance | <10 EU/mL |
| Purity | CBG COH QC | CD56(+) ≥80%; |
| | | CD3(+) ≤10% |
| Viability at Thaw | CBG COH QC | ≥70% |
| Viable Cell Concentration | CBG COH QC | Report Result |
| at Thaw | | |
| Replication Competent | Indiana | Not Detected |
| Retrovirus (for RD114 | University | |
| pseudotyped) | | |
| VCN Assay | COH APCF Lab | ≤5.0 Copies Per Cell |
| PDL1 Expression (FACS | CBG COH QC | ≥10% |
| Assay) | | |
| tEGFR Expression (FACS | CBG COH QC | ≥10% |
| Assay) | | |
| CD137/IL21 Feeder Cell | CBG COH QC | Residual K562 |
| Residual Assay | | Feeder Cells <10% |
| (FACS Assay) | | |
| IL-15 ELISA | CBG COH QC | Detectable IL-15, >1 |
| | | pg/ml |

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

“Truncated epidermal growth factor” or “truncated EGFR” or “tEGFR” refers to epidermal growth factor that is devoid of intracellular receptor tyrosine kinase activity. In embodiments, tEGFR is devoid of extracellular N-terminal ligand binding domains and intracellular receptor tyrosine kinase activity. In embodiments, tEGFR is devoid of extracellular N-terminal ligand binding domains and intracellular receptor tyrosine kinase activity, but retains the native amino acid sequence, type I transmembrane cell surface localization, and a conformationally intact binding epitope. In embodiments, tEGFR comprises EGFR Domain III, the EGFR transmembrane domain, and the EGFR Domain IV. In embodiments, tEGFR has at least 85% sequence identity to the protein identified by UniProt reference number Q9H3C8. In embodiments, tEGFR has at least 90% sequence identity to the protein identified by UniProt reference number Q9H3C8. In embodiments, tEGFR has at least 95% sequence identity to the protein identified by UniProt reference number Q9H3C8. In embodiments, tEGFR has the sequence set for the protein identified by UniProt reference number Q9H3C8.

A “PD-L1 protein” or “PD-L1” as referred to herein includes any of the recombinant or naturally-occurring forms of the programmed death-ligand 1 (PD-L1) also known as cluster of differentiation 274 (CD 274) or variants or homologs thereof that maintain PD-L1 protein activity (e.g. within at least 50%, 80%, 90%, 95%, 96%, 97%, 98%, 99% or 100% activity compared to PD-L1 protein). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto. In embodiments, the PD-L1 protein is substantially identical to the protein identified by the UniProt reference number Q9EP73 or a variant or homolog having substantial identity thereto.

“PD-1 pathway inhibitor” as provided herein refers to a substance capable of detectably lowering expression of or activity level of the PD-1 signaling pathway compared to a control. The inhibited expression or activity of the PD-1 signaling pathway can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “inhibitor” is a compound or small molecule that inhibits the PD-1 signaling pathway e.g., by binding, partially or totally blocking stimulation of the PD-1 pathway, decrease, prevent, or delay activation of the PD-1 pathway, or inactivate, desensitize, or down-regulate signal transduction, gene expression or enzymatic activity of the PD-1 pathway. In embodiments, the PD-1 pathway inhibitor inhibits PD-1 activity or expression. In embodiments, the PD-1 pathway inhibitor is a compound or a small molecule. In embodiments, the PD-1 pathway inhibitor is an antibody. In embodiments, the PD-1 pathway inhibitor is a programmed death-ligand 1 (PD-L1) inhibitor or a PD-1 inhibitor. A PD-L1 inhibitor as provided herein is a substance that, at least in part, partially or totally blocks stimulation, decreases, prevents, or delays activation, or inactivates, desensitizes, or down-regulates signal transduction of PD-1. A PD-1 inhibitor as provided herein is a substance that, at least in part, partially or totally blocks stimulation, decreases, prevents, or delays activation, or inactivates, desensitizes, or down-regulates signal transduction of PD-1.

“Anti-PD-1 refractory subject” or “refractory subject” refer to cancer patients who are unresponsive to PD-1 pathway inhibitor therapy, such as treatment with PD-1 inhibitors and/or PD-L1 inhibitors.

The term “cancer” is used in accordance with its plain ordinary meaning and refers to all types of cancer, neoplasm or malignant tumors found in mammals (e.g. humans), including leukemias, lymphomas, carcinomas and sarcomas. Examples of cancers that may be treated with a compound, composition, or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is lung cancer. In embodiments, the cancer is leukemia.

The term “leukemia” is used in accordance with its plain ordinary meaning and refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Examples of leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia. In embodiments, the cancer is acute myeloid leukemia.

The term “patient” or “subject in need thereof” is used in accordance with its plain ordinary meaning and refers to a living organism suffering from or prone to a disease or condition that can be treated by administration of a composition, compound, or method as provided herein. Non-limiting examples include humans, other mammals, bovines, rats, mice, dogs, cats, monkeys, goat, sheep, cows, and other non-mammalian animals. In embodiments, a patient is human. In embodiments, the subject has, had, or is suspected of having cancer.

The terms “control” or “control experiment” are used in accordance with its plain ordinary meaning and refer to an experiment in which the subjects or reagents of the experiment are treated as in a parallel experiment except for omission of a procedure, reagent, or variable of the experiment. In some instances, the control is used as a standard of comparison in evaluating experimental effects. In embodiments, a control is the measurement of the activity of a protein in the absence of a compound as described herein (including embodiments and examples).

The terms “treating” or “treatment” are used in accordance with its plain ordinary meaning and refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination. Treating does not include preventing.

By “an effective amount,” “a therapeutically effective amount,” “therapeutically effective dose or amount” and the like is intended an amount of cells, agents, or compounds described herein that brings about a positive therapeutic response in a subject in need of, such as an amount that restores function and/or results in the elimination and/or reduction of tumor and/or cancer cells. The exact amount (of cells or agents) required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein. A “combined therapeutically effective amount” or “combined therapeutically effective dose or amount dose” refers a combination of therapies that together brings about a positive therapeutic response in a subject in need of, such as an amount that restores function and/or results in the elimination and/or reduction of tumor and/or cancer cells.

The term “immune response” is used in accordance with its plain ordinary meaning and refers to a response by an organism that protects against disease. The response can be mounted by the innate immune system or by the adaptive immune system.

The term “T cells” or “T lymphocytes” are used in accordance with their plain ordinary meaning and refer to a type of lymphocyte (a subtype of white blood cell) involved in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

The terms “tumor microenvironment”, “TME”, and “cancer microenvironment” are used in accordance with its plain ordinary meaning and refer to the non-neoplastic cellular environment of a tumor, including blood vessels, immune cells, fibroblasts, cytokines, chemokines, non-cancerous cells present in the tumor, and proteins produced

The terms “activation”, “activate”, “activating”, “activator” and the like are used in accordance with its plain ordinary meaning and refer to an interaction that positively affects (e.g. increasing) the activity or function of a protein or cell relative to the activity or function of the protein or cell in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein that is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. are used in accordance with its plain ordinary meaning and refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In embodiments, expression or activity is 1.5-fold, 2-fold, 3-fold, 4-fold, 5-fold, 10-fold or higher than the expression or activity in the absence of the agonist.

The terms “inhibition,” “inhibit,” “inhibiting,” and the like are used in accordance with its plain ordinary meaning and refer to an interaction that negatively affecting (e.g. decreasing) the activity or function of the protein or cell relative to the activity or function of the protein or cell in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein or cell from an indirect interaction (e.g. an inhibitor binds to a protein that activates the target protein, thereby preventing target protein activation or cell activations).

The terms “inhibitor,” “repressor” or “antagonist” or “downregulator” are used in accordance with its plain ordinary meaning and refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In embodiments, expression or activity is 1.5-fold, 2-fold, 3-fold, 4-fold, 5-fold, 10-fold or lower than the expression or activity in the absence of the antagonist.

The term “expression” is used in accordance with its plain ordinary meaning and refers to any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “signaling pathway” is used in accordance with its plain ordinary meaning and refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “cytokine” is used in accordance with its plain ordinary meaning and refers to a broad category of small proteins that are important in cell signaling. Cytokines are peptides, and cannot cross the lipid bilayer of cells to enter the cytoplasm. Cytokines are involved in autocrine signaling, paracrine signaling and endocrine signaling as immunomodulating agents. Cytokines include chemnokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell.

The terms “IFN-γ” and “interferon gamma” are used herein according to its plain and ordinary meaning and refer to a dimerized soluble cytokine that is the only member of the type II class of interferons. It plays a role in innate and adaptive immunity against viral, some bacterial and protozoal infections. IFNγ is an important activator of macrophages and inducer of Class II major histocompatibility complex (MHC) molecule expression. The importance of IFNγ in the immune system stems in part from its ability to inhibit viral replication directly and from its immunostimulatory and immunomodulatory effects. IFNγ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops.

The terms “CD107α,” “CD107-alpha,” “lysosomal-associated membrane protein 1,” “LAMP-1,” and “lysosome-associated glycoprotein 1” are used in accordance with their plain ordinary meaning and refer to a glycoprotein from a family of lysosome-associated membrane glycoproteins. CD107α is a type I transmembrane protein which is expressed at high or medium levels in at least 76 different normal tissue cell types. It resides primarily across lysosomal membranes, and functions to provide selectins with carbohydrate ligands. CD107α has also been shown to be a marker of degranulation on lymphocytes such as CD8+ and NK cells.

The terms “IL-12,” “IL12,” and “interleukin-12” are used in accordance with their plain ordinary meaning and refer to an interleukin that is naturally produced by dendritic cells, macrophages, neutrophils, and human B-lymphoblastoid cells in response to antigenic stimulation plays an important role in the activities of natural killer cells and T lymphocytes. IL-12 mediates enhancement of the cytotoxic activity of NK cells and CD8+ cytotoxic T lymphocytes. There may be a link between IL-2 and the signal transduction of IL-12 in NK cells. IL-2 stimulates the expression of two IL-12 receptors, IL-12R-01 and IL-12R-02, maintaining the expression of a critical protein involved in IL-12 signaling in NK cells. Enhanced functional response is demonstrated by IFN-γ production and killing of target cells.

The terms “IL-15” “interleukin-15,” and “IL15” are used in accordance with their plain ordinary meaning and refer to a cytokine with structural similarity to interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells. As a pleiotropic cytokine, it plays an important role in innate and adaptive immunity.

The terms “soluble IL-15”, “sIL-15”, and “sIL15” refer to an IL-15 protein capable of being secreted by an NK cell. In embodiments, the soluble IL-15 includes an IL-2 amino acid signal sequence to facilitate NK cell secretion and/or increase NK cell secretion of the IL-15 relative to the absence of the IL-2 amino acid signal sequence. In embodiments, the soluble IL-15 includes an IL-2 amino acid signal sequence encoded by a nucleic acid including the nucleotide sequence of SEQ ID NO: 1. In embodiments, the soluble IL-15 includes an IL-2 amino acid signal sequence encoded by a nucleic acid that is the nucleotide sequence of SEQ ID NO:1. In embodiments, the soluble IL-15 includes an IL-2 amino acid signal sequence encoded by a nucleic acid that has at least 90%, 95%, 96%, 97%, 98%, 99%, or 100% nucleotide sequence identity across the whole nucleic acid sequence or a portion of the nucleic acid sequence (e.g. a 10, 20, or 30 continuous nucleotide portion) compared to a naturally occurring nucleic acid encoding an IL-2 signaling sequence. In embodiments, the soluble IL-15 includes an IL-2 amino acid signal sequence encoded by a nucleic acid that has at least 90%, 95%, 96%, 97%, 98%, 99%, or 100% nucleotide sequence identity across the whole nucleic acid or a portion of the nucleic acid sequence (e.g. a 10, 20, or 30 continuous nucleotide portion) compared to the nucleic acid sequence of SEQ ID NO:1. In embodiments, the soluble IL-15 includes an IL-15 amino acid signal sequence to facilitate NK cell secretion. In embodiments, the soluble IL-15 includes an IL-15 amino acid protein sequence. In embodiments, the soluble IL-15 includes an IL-15 amino acid protein sequence encoded by a nucleic acid including the nucleotide sequence of SEQ ID NO:2. In embodiments, the soluble IL-15 includes an IL-15 amino acid protein sequence encoded by a nucleic acid that is the nucleotide sequence of SEQ ID NO:2. In embodiments, the soluble IL-15 includes an IL-15 amino acid protein sequence encoded by a nucleic acid that has at least 90%, 95%, 96%, 97%, 98%, 99%, or 100% nucleotide sequence identity across the whole nucleic acid or a portion of the nucleic acid sequence (e.g. a 10, 20, or 30 continuous nucleotide portion) compared to the nucleic acid sequence of SEQ ID NO:2. In embodiments, the soluble IL-15 is encoded by a nucleic acid including the nucleotide sequence of SEQ ID NO:3. In embodiments, the soluble IL-15 is encoded by a nucleic acid that is the nucleotide sequence of SEQ ID NO:3. In embodiments, the soluble IL-15 is encoded by a nucleic acid that has at least 90%, 95%, 96%, 97%, 98%, 99%, or 100% nucleotide sequence identity across the whole nucleic acid or a portion of the nucleic acid sequence (e.g. a 10, 20, or 30 continuous nucleotide portion) compared to the nucleic acid sequence of SEQ ID NO:3.

In embodiments, the soluble IL-15 includes an IL-2 amino acid signal sequence including the amino acid sequence of SEQ ID NO:4. In embodiments, the soluble IL-15 includes an IL-2 amino acid signal sequence that is the amino acid sequence of SEQ ID NO:4. In embodiments, the soluble IL-15 includes an IL-2 amino acid signal sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to a naturally occurring IL-2 signaling sequence. In embodiments, the soluble IL-15 includes an IL-2 amino acid signal sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to the amino acid sequence of SEQ ID NO:4. In embodiments, the soluble IL-15 includes an IL-15 amino acid protein sequence including the amino acid sequence of SEQ ID NO:5. In embodiments, the soluble IL-15 includes an IL-15 amino acid protein sequence that is the amino acid sequence of SEQ ID NO:5. In embodiments, the soluble IL-15 includes an IL-15 amino acid protein sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to a naturally occurring IL-15 protein sequence. In embodiments, the soluble IL-15 includes an IL-15 amino acid protein sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to the amino acid sequence of SEQ ID NO:5. In embodiments, the soluble IL-15 includes the amino acid sequence of SEQ ID NO:6. In embodiments, the soluble IL-15 is the amino acid sequence of SEQ ID NO:6. In embodiments, the soluble IL-15 includes an amino acid sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to the amino acid sequence of SEQ ID NO:6.

The terms “IL-18,” “interleukin-18,” “IL18,” and “interferon-gamma inducing factor” are used in accordance with their plain ordinary meaning and refer to a proinflammatory cytokine that belongs to the IL-1 superfamily and is produced by macrophages and other cells. IL-18 works by binding to the interleukin-18 receptor, and together with IL-12, it induces cell-mediated immunity following infection with microbial products like lipopolysaccharide (LPS). After stimulation with IL-18, natural killer (NK) cells and certain T cells release another important cytokine called interferon-γ (IFN-γ) or type II interferon that plays an important role in activating the macrophages or other cells.

The term “immunotherapy,” “immunotherapeutic,” and “immunotherapeutic agent” are used in accordance with their plain ordinary meaning and refer to the treatment of disease by activating or suppressing the immune system. Immunotherapies designed to elicit or amplify an immune response are classified as activation immunotherapies, while immunotherapies that reduce or suppress are classified as suppression immunotherapies. Such immunotherapeutic agents include antibodies and cell therapy.

The term “checkpoint inhibitor” is used in accordance with its plain ordinary meaning and refers to a drug, often made of antibodies, that unleashes an immune system attack on cancer cells. An important part of the immune system is its ability to tell between normal cells in the body and those it sees as “foreign.” This lets the immune system attack the foreign cells while leaving the normal cells alone. To do this, it uses “checkpoints” which are molecules on certain immune cells that need to be activated (or inactivated) to start an immune response. Cancer cells sometimes find ways to use these checkpoints to avoid being attacked by the immune system. Drugs that target these checkpoints are known as checkpoint inhibitors.

The term “feeder cell” or “feeders” are used in accordance with their plain ordinary meaning and refer to adherent growth-arrested, but viable and bioactive, cells. These cells may be used as a substratum to condition the medium on which other cells, particularly at low or clonal density, are grown. In embodiments, the cells of the feeder layer are irradiated or otherwise treated so that they will not proliferate.

The terms “K562 cell” and “K562 cell line” are used in accordance with their plain ordinary meaning and refer to a human immortalized myelogenous leukemia cell line derived from a 53-year-old female chronic myelogenous leukemia patient in blast crisis. K562 cells are of the erythroleukemia type. The cells are non-adherent and rounded, are positive for the BCR:ABL fusion gene, and bear some proteomic resemblance to both undifferentiated granulocytes and erythrocytes.

The term “anticancer agent” and “anticancer therapy” are used in accordance with their plain ordinary meaning and refer to a molecule or composition (e.g. compound, peptide, protein, nucleic acid, drug, antagonist, inhibitor, modulator) or regimen used to treat cancer through destruction or inhibition of cancer cells or tissues. Anticancer therapy includes chemotherapy, radiation therapy, surgery, targeted therapy, immunotherapy, and cell therapy Anticancer agents and/or anticancer therapy may be selective for certain cancers or certain tissues. In embodiments, an anti-cancer therapy is an immunotherapy. In embodiments, anticancer agent or therapy may include a checkpoint inhibitor (e.g. administration of an effective amount of a checkpoint inhibitor). In embodiments, the anti-cancer agent or therapy is a cell therapy.

In embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD-184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD-184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stemcell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; elfomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL.sub.2), interferon alfa-2a; interferon alfa-2b; interferon alfa-n1; interferon alfa-n3; interferon beta-1a; interferon gamma-1b; iproplatin; irnnotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol (i.e. paclitaxel), Taxotere, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD-153035, BMS-599626), sorafenib, imatinib, sunitinib, dasatinib, pembrolizumab nivolumab, atezolizumab, avelumab, durvalumab or the like.

The terms “cell therapy” and “cellular therapy” are used in accordance with their plain ordinary meaning and refer to therapy in which cellular material such as for example cells is injected, grafted or implanted into a patient. The cells may be living cells. In embodiments, the cells are NK cells expressing PD-L1 protein.

The terms “HLA,” “HLA type,” “human leukocyte antigen system,” and “human leukocyte antigen complex” are used in accordance with their plain ordinary meaning and refer to a gene complex encoding the major histocompatibility complex (MHC) proteins in humans. These cell-surface proteins are responsible for the regulation of the immune system in humans.

### PD-L1(+) Natural Killer Cells

Provided herein are PD-L1(+) natural killer cells. In embodiments, the PD-L1(+) natural killer cells express cell surface PD-L1. In embodiments, the PD-L1(+) natural killer cells express soluble IL-15. In embodiments, the PD-L1(+) natural killer cells include a nucleic acid encoding the soluble IL-15. In embodiments, the nucleic acid includes the nucleotide sequence of SEQ ID NO: 1. In embodiments, the nucleic acid includes the nucleotide sequence of SEQ ID NO:2. In embodiments, the nucleic acid includes the nucleotide sequence of SEQ ID NO:3. In embodiments, the nucleic acid is the nucleotide sequence of SEQ ID NO:3. In embodiments, the soluble IL-15 includes the amino acid sequence of SEQ ID NO:4. In embodiments, the soluble IL-15 includes the amino acid sequence of SEQ ID NO:5. In embodiments, the soluble IL-15 includes the amino acid sequence of SEQ ID NO:6. In embodiments, the soluble IL-15 is the amino acid sequence of SEQ ID NO:6. In embodiments, the PD-L1(+) natural killer cells express truncated EGFR. In embodiments, the PD-L1(+) natural killer cells express soluble IL-15 and truncated EGFR. In embodiments, the PD-L1(+) natural killer cells express cell surface PD-L1 and soluble IL-15. In embodiments, the PD-L1(+) natural killer cells express cell surface PD-L1 and truncated EGFR. In embodiments, the PD-L1(+) natural killer cells express cell surface PD-L1, soluble IL-15, and truncated EGFR. In embodiments, the PD-L1(+) natural killer cells constitutively express cell surface PD-L1, soluble IL-15, and truncated EGFR. In embodiments, the PD-L1(+) natural killer cells do not express a CD19 chimeric antigen receptor. In embodiments, the PD-L1(+) natural killer cells were derived from umbilical cord blood natural killer cells. In embodiments, the umbilical cord blood natural killer cells were incubated with IL-12 and IL-18. In embodiments, the PD-L1(+) natural killer cells do not express a CD19 chimeric antigen receptor. In embodiments, the umbilical cord blood natural killer cells were incubated with IL-12 and IL-18, and do not express a CD19 chimeric antigen receptor. In embodiments, the PD-L1(+) natural killer cells are recombinant PD-L1(+) natural killer cells.

Provided herein are a population of PD-L1(+) natural killer cells. In embodiments, the population of PD-L1(+) natural killer cells express cell surface PD-L1. In embodiments, the population of PD-L1(+) natural killer cells express soluble IL-15. In embodiments, the population of PD-L1(+) natural killer cells include a nucleic acid encoding the soluble IL-15. In embodiments, the nucleic acid includes the nucleotide sequence of SEQ ID NO:1. In embodiments, the nucleic acid includes the nucleotide sequence of SEQ ID NO:2. In embodiments, the nucleic acid includes the nucleotide sequence of SEQ ID NO:3. In embodiments, the nucleic acid is the nucleotide sequence of SEQ ID NO:3. In embodiments, the soluble IL-15 includes the amino acid sequence of SEQ ID NO:4 In embodiments, the soluble IL-15 includes the amino acid sequence of SEQ ID NO:5. In embodiments, the soluble IL-15 includes the amino acid sequence of SEQ ID NO:6. In embodiments, the soluble IL-15 is the amino acid sequence of SEQ ID NO:6. In embodiments, the population of PD-L1(+) natural killer cells express truncated EGFR. In embodiments, the population of PD-L1(+) natural killer cells express soluble IL-15 and truncated EGFR. In embodiments, the population of PD-L1(+) natural killer cells express cell surface PD-L1 and soluble IL-15. In embodiments, the population of PD-L1(+) natural killer cells express cell surface PD-L1 and truncated EGFR. In embodiments, the population of PD-L1(+) natural killer cells express cell surface PD-L1, soluble IL-15, and truncated EGFR. In embodiments, the population of PD-L1(+) natural killer cells constitutively express cell surface PD-L1, soluble IL-15, and truncated EGFR. In embodiments, the population of PD-L1(+) natural killer cells do not express a CD19 chimeric antigen receptor. In embodiments, the population of PD-L1(+) natural killer cells were derived from umbilical cord blood natural killer cells. In embodiments, the umbilical cord blood natural killer cells were incubated with IL-12 and IL-18. In embodiments, the population of PD-L1(+) natural killer cells do not express a CD19 chimeric antigen receptor. In embodiments, the umbilical cord blood natural killer cells were incubated with IL-12 and IL-18, and do not express a CD19 chimeric antigen receptor. In embodiments, the population of PD-L1(+) natural killer cells are a population of recombinant PD-L1(+) natural killer cells.

Provided herein are activated cord blood PD-L1(+) natural killer cells. In embodiments, the activated cord blood PD-L1(+) natural killer cells express cell surface PD-L1. In embodiments, the activated cord blood PD-L1(+) natural killer cells express soluble IL-15. In embodiments, the activated cord blood PD-L1(+) natural killer cells include a nucleic acid encoding the soluble IL-15. In embodiments, the nucleic acid includes the nucleotide sequence of SEQ ID NO:1. In embodiments, the nucleic acid includes the nucleotide sequence of SEQ ID NO:2. In embodiments, the nucleic acid includes the nucleotide sequence of SEQ ID NO:3. In embodiments, the nucleic acid is the nucleotide sequence of SEQ ID NO:3. In embodiments, the soluble IL-15 includes the amino acid sequence of SEQ ID NO:4. In embodiments, the soluble IL-15 includes the amino acid sequence of SEQ ID NO:5. In embodiments, the soluble IL-15 includes the amino acid sequence of SEQ ID NO:6. In embodiments, the soluble IL-15 is the amino acid sequence of SEQ ID NO:6. In embodiments, the activated cord blood PD-L1(+) natural killer cells express truncated EGFR. In embodiments, the activated cord blood PD-L1(+) natural killer cells express soluble IL-15 and truncated EGFR. In embodiments, the activated cord blood PD-L1(+) natural killer cells express cell surface PD-L1, soluble IL-15 and truncated EGFR. In embodiments, the activated cord blood PD-L1(+) natural killer cells constitutively express cell surface PD-L1, soluble IL-15, and truncated EGFR. In embodiments, the activated cord blood PD-L1(+) natural killer cells do not express a CD19 chimeric antigen receptor. In embodiments, the PD-L1(+) natural killer cells were derived from umbilical cord blood natural killer cells. In embodiments, the umbilical cord blood natural killer cells were incubated with IL-12 and IL-18. In embodiments, the PD-L1(+) natural killer cells do not express a CD19 chimeric antigen receptor. In embodiments, the umbilical cord blood natural killer cells were incubated with IL-12 and IL-18, and do not express a CD19 chimeric antigen receptor. In embodiments, the PD-L1(+) natural killer cells are recombinant PD-L1(+) natural killer cells.

Provided herein are a population of activated cord blood PD-L1(+) natural killer cells. In embodiments, the population of activated cord blood PD-L1(+) natural killer cells express cell surface PD-L1. In embodiments, the population of activated cord blood PD-L1(+) natural killer cells express soluble IL-15. In embodiments, the population of activated cord blood PD-L1(+) natural killer cells include a nucleic acid encoding the soluble IL-15. In embodiments, the nucleic acid includes the nucleotide sequence of SEQ ID NO:1. In embodiments, the nucleic acid includes the nucleotide sequence of SEQ ID NO:2. In embodiments, the nucleic acid includes the nucleotide sequence of SEQ ID NO:3. In embodiments, the nucleic acid is the nucleotide sequence of SEQ ID NO:3. In embodiments, the soluble IL-15 includes the amino acid sequence of SEQ ID NO:4. In embodiments, the soluble IL-15 includes the amino acid sequence of SEQ ID NO:5. In embodiments, the soluble IL-15 includes the amino acid sequence of SEQ ID NO:6. In embodiments, the soluble IL-15 is the amino acid sequence of SEQ ID NO:6. In embodiments, the population of activated cord blood PD-L1(+) natural killer cells express truncated EGFR. In embodiments, the population of activated cord blood PD-L1(+) natural killer cells express soluble IL-15 and truncated EGFR. In embodiments, the population of activated cord blood PD-L1(+) natural killer cells express cell surface PD-L1, soluble IL-15 and truncated EGFR. In embodiments, the population of activated cord blood PD-L1(+) natural killer cells constitutively express cell surface PD-L1, soluble IL-15, and truncated EGFR. In embodiments, the population of activated cord blood PD-L1(+) natural killer cells do not express a CD19 chimeric antigen receptor. In embodiments, the population of PD-L1(+) natural killer cells were derived from umbilical cord blood natural killer cells. In embodiments, the umbilical cord blood natural killer cells were incubated with IL-12 and IL-18. In embodiments, the population of PD-L1(+) natural killer cells do not express a CD19 chimeric antigen receptor. In embodiments, the population of umbilical cord blood natural killer cells were incubated with IL-12 and IL-18, and do not express a CD19 chimeric antigen receptor. In embodiments, the population of PD-L1(+) natural killer cells are a population of recombinant PD-L1(+) natural killer cells.

Provided herein are a population of natural killer cells, wherein at least 40% of the population of nature killer cells are PD-L1(+) natural killer cells. In embodiments, at least 45% of the population of nature killer cells are PD-L1(+) natural killer cells. In embodiments, at least 50% of the population of nature killer cells are PD-L1(+) natural killer cells. In embodiments, at least 55% of the population of nature killer cells are PD-L1(+) natural killer cells. In embodiments, at least 60% of the population of nature killer cells are PD-L1(+) natural killer cells. In embodiments, at least 65% of the population of nature killer cells are PD-L1(+) natural killer cells. In embodiments, at least 70% of the population of nature killer cells are PD-L1(+) natural killer cells. In embodiments, at least 75% of the population of nature killer cells are PD-L1(+) natural killer cells. In embodiments, at least 80% of the population of nature killer cells are PD-L1(+) natural killer cells. In embodiments, at least 85% of the population of nature killer cells are PD-L1(+) natural killer cells. In embodiments, at least 90% of the population of nature killer cells are PD-L1(+) natural killer cells. In embodiments, at least 95% of the population of nature killer cells are PD-L1(+) natural killer cells. In embodiments, about 40% to about 90% of the population of nature killer cells are PD-L1(+) natural killer cells. In embodiments, about 45% to about 90% of the population of nature killer cells are PD-L1(+) natural killer cells. In embodiments, about 50% to about 90% of the population of nature killer cells are PD-L1(+) natural killer cells. In embodiments, about 55% to about 90% of the population of nature killer cells are PD-L1(+) natural killer cells. In embodiments, about 60% to about 90% of the population of nature killer cells are PD-L1(+) natural killer cells. In embodiments, about 65% to about 90% of the population of nature killer cells are PD-L1(+) natural killer cells. In embodiments, about 70% to about 90% of the population of nature killer cells are PD-L1(+) natural killer cells. In embodiments, about 75% to about 90% of the population of nature killer cells are PD-L1(+) natural killer cells. In embodiments, about 80% to about 90% of the population of nature killer cells are PD-L1(+) natural killer cells

Provided herein are a population of natural killer cells, wherein at least 40% of the population of nature killer cells are PD-L1(+) natural killer cells that express soluble IL-15. In embodiments, at least 45% of the population of nature killer cells are PD-L1(+) natural killer cells that express soluble IL-15. In embodiments, at least 50% of the population of nature killer cells are PD-L1(+) natural killer cells that express soluble IL-15. In embodiments, at least 55% of the population of nature killer cells are PD-L1(+) natural killer cells that express soluble IL-15. In embodiments, at least 60% of the population of nature killer cells are PD-L1(+) natural killer cells that express soluble IL-15. In embodiments, at least 65% of the population of nature killer cells are PD-L1(+) natural killer cells that express soluble IL-15. In embodiments, at least 70% of the population of nature killer cells are PD-L1(+) natural killer cells that express soluble IL-15. In embodiments, at least 75% of the population of nature killer cells are PD-L1(+) natural killer cells that express soluble IL-15. In embodiments, at least 80% of the population of nature killer cells are PD-L1(+) natural killer cells that express soluble IL-15. In embodiments, at least 85% of the population of nature killer cells are PD-L1(+) natural killer cells that express soluble IL-15. In embodiments, at least 90% of the population of nature killer cells are PD-L1(+) natural killer cells that express soluble IL-15. In embodiments, at least 95% of the population of nature killer cells are PD-L1(+) natural killer cells that express soluble IL-15. In embodiments, about 40% to about 95% of the population of nature killer cells are PD-L1(+) natural killer cells that express soluble IL-15. In embodiments, about 45% to about 95% of the population of nature killer cells are PD-L1(+) natural killer cells that express soluble IL-15. In embodiments, about 50% to about 95% of the population of nature killer cells are PD-L1(+) natural killer cells that express soluble IL-15. In embodiments, about 55% to about 95% of the population of nature killer cells are PD-L1(+) natural killer cells that express soluble IL-15. In embodiments, about 60% to about 95% of the population of nature killer cells are PD-L1(+) natural killer cells that express soluble IL-15. In embodiments, about 65% to about 95% of the population of nature killer cells are PD-L1(+) natural killer cells that express soluble IL-15. In embodiments, about 70% to about 95% of the population of nature killer cells are PD-L1(+) natural killer cells that express soluble IL-15. In embodiments, about 75% to about 95% of the population of nature killer cells are PD-L1(+) natural killer cells that express soluble IL-15. In embodiments, about 80% to about 95% of the population of nature killer cells are PD-L1(+) natural killer cells that express soluble IL-15. In embodiments, about 85% to about 95% of the population of nature killer cells are PD-L1(+) natural killer cells that express soluble IL-15. In embodiments, about 90% to about 95% of the population of nature killer cells are PD-L1(+) natural killer cells that express soluble IL-15. In embodiments, about 40% to about 90% of the population of nature killer cells are PD-L1(+) natural killer cells that express soluble IL-15. In embodiments, about 45% to about 90% of the population of nature killer cells are PD-L1(+) natural killer cells that express soluble IL-15. In embodiments, about 50% to about 90% of the population of nature killer cells are PD-L1(+) natural killer cells that express soluble IL-15. In embodiments, about 55% to about 90% of the population of nature killer cells are PD-L1(+) natural killer cells that express soluble IL-15. In embodiments, about 60% to about 90% of the population of nature killer cells are PD-L1(+) natural killer cells that express soluble IL-15. In embodiments, about 65% to about 90% of the population of nature killer cells are PD-L1(+) natural killer cells that express soluble IL-15. In embodiments, about 70% to about 90% of the population of nature killer cells are PD-L1(+) natural killer cells that express soluble IL-15. In embodiments, about 75% to about 90% of the population of nature killer cells are PD-L1(+) natural killer cells that express soluble IL-15. In embodiments, about 80% to about 90% of the population of nature killer cells are PD-L1(+) natural killer cells that express soluble IL-15. In embodiments, about 85% to about 90% of the population of nature killer cells are PD-L1(+) natural killer cells that express soluble IL-15. In embodiments, at least 40% of the population of nature killer cells are PD-L1(+) natural killer cells that express soluble IL-15 and truncated EGFR. In embodiments, at least 45% of the population of nature killer cells are PD-L1(+) natural killer cells that express soluble IL-15 and truncated EGFR. In embodiments, at least 50% of the population of nature killer cells are PD-L1(+) natural killer cells that express soluble IL-15 and truncated EGFR. In embodiments, at least 55% of the population of nature killer cells are PD-L1(+) natural killer cells that express soluble IL-15 and truncated EGFR. In embodiments, at least 60% of the population of nature killer cells are PD-L1(+) natural killer cells that express soluble IL-15 and truncated EGFR. In embodiments, at least 65% of the population of nature killer cells are PD-L1(+) natural killer cells that express soluble IL-15 and truncated EGFR. In embodiments, at least 70% of the population of nature killer cells are PD-L1(+) natural killer cells that express soluble IL-15 and truncated EGFR. In embodiments, at least 75% of the population of nature killer cells are PD-L1(+) natural killer cells that express soluble IL-15 and truncated EGFR. In embodiments, at least 80% of the population of nature killer cells are PD-L1(+) natural killer cells that express soluble IL-15 and truncated EGFR. In embodiments, at least 85% of the population of nature killer cells are PD-L1(+) natural killer cells that express soluble IL-15 and truncated EGFR. In embodiments, at least 90% of the population of nature killer cells are PD-L1(+) natural killer cells that express soluble IL-15 and truncated EGFR. In embodiments, at least 95% of the population of nature killer cells are PD-L1(+) natural killer cells that express soluble IL-15 and truncated EGFR. In embodiments, about 40% to about 95% of the population of nature killer cells are PD-L1(+) natural killer cells that express soluble IL-15 and truncated EGFR. In embodiments, about 45% to about 95% of the population of nature killer cells are PD-L1(+) natural killer cells that express soluble IL-15 and truncated EGFR. In embodiments, about 50% to about 95% of the population of nature killer cells are PD-L1(+) natural killer cells that express soluble IL-15 and truncated EGFR. In embodiments, about 55% to about 95% of the population of nature killer cells are PD-L1(+) natural killer cells that express soluble IL-15 and truncated EGFR. In embodiments, about 60% to about 95% of the population of nature killer cells are PD-L1(+) natural killer cells that express soluble IL-15 and truncated EGFR. In embodiments, about 65% to about 95% of the population of nature killer cells are PD-L1(+) natural killer cells that express soluble IL-15 and truncated EGFR. In embodiments, about 70% to about 95% of the population of nature killer cells are PD-L1(+) natural killer cells that express soluble IL-15 and truncated EGFR. In embodiments, about 75% to about 95% of the population of nature killer cells are PD-L1(+) natural killer cells that express soluble IL-15 and truncated EGFR. In embodiments, about 80% to about 95% of the population of nature killer cells are PD-L1(+) natural killer cells that express soluble IL-15 and truncated EGFR. In embodiments, about 85% to about 95% of the population of nature killer cells are PD-L1(+) natural killer cells that express soluble IL-15 and truncated EGFR. In embodiments, about 90% to about 95% of the population of nature killer cells are PD-L1(+) natural killer cells that express soluble IL-15 and truncated EGFR. In embodiments, about 40% to about 90% of the population of nature killer cells are PD-L1(+) natural killer cells that express soluble IL-15 and truncated EGFR. In embodiments, about 45% to about 90% of the population of nature killer cells are PD-L1(+) natural killer cells that express soluble IL-15 and truncated EGFR. In embodiments, about 50% to about 90% of the population of nature killer cells are PD-L1(+) natural killer cells that express soluble IL-15 and truncated EGFR. In embodiments, about 55% to about 90% of the population of nature killer cells are PD-L1(+) natural killer cells that express soluble IL-15 and truncated EGFR. In embodiments, about 60% to about 90% of the population of nature killer cells are PD-L1(+) natural killer cells that express soluble IL-15 and truncated EGFR. In embodiments, about 65% to about 90% of the population of nature killer cells are PD-L1(+) natural killer cells that express soluble IL-15 and truncated EGFR. In embodiments, about 70% to about 90% of the population of nature killer cells are PD-L1(+) natural killer cells that express soluble IL-15 and truncated EGFR. In embodiments, about 75% to about 90% of the population of nature killer cells are PD-L1(+) natural killer cells that express soluble IL-15 and truncated EGFR. In embodiments, about 80% to about 90% of the population of nature killer cells are PD-L1(+) natural killer cells that express soluble IL-15 and truncated EGFR. In embodiments, about 85% to about 90% of the population of nature killer cells are PD-L1(+) natural killer cells that express soluble IL-15 and truncated EGFR. In embodiments, the PD-L1(+) natural killer cells are activated cord blood PD-L1(+) natural killer cells. In embodiments, the PD-L1(+) natural killer cells constitutively express soluble IL-15. In embodiments, the PD-L1(+) natural killer cells constitutively express soluble IL-15 and truncated EGFR. In embodiments, the PD-L1(+) natural killer cells that have been genetically modified to constitutively express soluble IL-15. In embodiments, the PD-L1(+) natural killer cells that have been genetically modified to constitutively express soluble IL-15 and truncated EGFR. In embodiments, the PD-L1(+) natural killer cells do not express a CD19 chimeric antigen receptor. In embodiments, the PD-L1(+) natural killer cells were derived from umbilical cord blood natural killer cells. In embodiments, the umbilical cord blood natural killer cells were incubated with IL-12 and IL-18. In embodiments, the umbilical cord blood natural killer cells were incubated with IL-12 and IL-18, and do not express a CD19 chimeric antigen receptor. In embodiments, the PD-L1(+) natural killer cells are recombinant PD-L1(+) natural killer cells

### Methods of Treatment

In embodiments, the disclosure provides methods of treating cancer in a patient in need thereof comprising administering to the patient an effective amount of PD-L1(+) natural killer cells. In embodiments, the PD-L1(+) natural killer cells expresses soluble IL-15. In embodiments, the PD-L1(+) natural killer cells include a nucleic acid encoding the soluble IL-15. In embodiments, the nucleic acid includes the nucleotide sequence of SEQ ID NO:1. In embodiments, the nucleic acid includes the nucleotide sequence of SEQ ID NO:2. In embodiments, the nucleic acid includes the nucleotide sequence of SEQ ID NO:3. In embodiments, the nucleic acid is the nucleotide sequence of SEQ ID NO: 3. In embodiments, the soluble IL-15 includes the amino acid sequence of SEQ ID NO:4 In embodiments, the soluble IL-15 includes the amino acid sequence of SEQ ID NO: 5. In embodiments, the soluble IL-15 includes the amino acid sequence of SEQ ID NO: 6. In embodiments, the soluble IL-15 is the amino acid sequence of SEQ ID NO:6. In embodiments, the PD-L1(+) natural killer cells express truncated EGFR. In embodiments, the PD-L1(+) natural killer cells express soluble IL-15 and truncated EGFR. In embodiments, the PD-L1(+) natural killer cells are an activated cord blood natural killer cell. In embodiments, the PD-L1(+) natural killer cells are referred to as a population of PD-L1(+) natural killer cells. In embodiments, the PD-L1(+) natural killer cells are recombinant PD-L1(+) natural killer cells. In embodiments, the cancer is lung cancer. In embodiments, the cancer is non-small cell lung cancer. In embodiments, the cancer is advanced non-small cell lung cancer. In embodiments, the cancer is metastatic non-small cell lung cancer. In embodiments, the cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the cancer is metastatic cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient an effective amount of PD-L1(+) natural killer cells about once per week. In embodiments, the disclosure provides methods of treating cancer in a patient in need thereof comprising administering to the patient an effective amount of PD-L1(+) natural killer cells about twice per week. In embodiments, the disclosure provides methods of treating cancer in a patient in need thereof comprising administering to the patient an effective amount of PD-L1(+) natural killer cells about once per day. In embodiments, the disclosure provides methods of treating cancer in a patient in need thereof comprising administering to the patient an effective amount of PD-L1(+) natural killer cells about once every two days. In embodiments, the disclosure provides methods of treating cancer in a patient in need thereof comprising administering to the patient an effective amount of PD-L1(+) natural killer cells about once every three days. In embodiments, the disclosure provides methods of treating cancer in a patient in need thereof comprising administering to the patient an effective amount of PD-L1(+) natural killer cells about once every four days. In embodiments, the disclosure provides methods of treating cancer in a patient in need thereof comprising administering to the patient an effective amount of PD-L1(+) natural killer cells about once every three or four days. In embodiments, the disclosure provides methods of treating cancer in a patient in need thereof comprising administering to the patient an effective amount of PD-L1(+) natural killer cells about once every five days. In embodiments, the disclosure provides methods of treating cancer in a patient in need thereof comprising administering to the patient an effective amount of PD-L1(+) natural killer cells about once every six days. In embodiments, the patient is administered from about 1×10⁶ to about 1×10¹² of the PD-L1(+) natural killer cells. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR. In embodiments, the cancer is lung cancer. In embodiments, the cancer is non-small cell lung cancer. In embodiments, the cancer is advanced non-small cell lung cancer. In embodiments, the cancer is metastatic non-small cell lung cancer. In embodiments, the cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the cancer is metastatic cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient an effective amount of PD-L1(+) natural killer cells about once per week; wherein the PD-L1(+) natural killer cells express soluble IL-15. In embodiments, the disclosure provides methods of treating cancer in a patient in need thereof comprising administering to the patient an effective amount of PD-L1(+) natural killer cells about twice per week; wherein the PD-L1(+) natural killer cells express soluble IL-15. In embodiments, the disclosure provides methods of treating cancer in a patient in need thereof comprising administering to the patient an effective amount of PD-L1(+) natural killer cells about once per day; wherein the PD-L1(+) natural killer cells express soluble IL-15. In embodiments, the disclosure provides methods of treating cancer in a patient in need thereof comprising administering to the patient an effective amount of PD-L1(+) natural killer cells about once every two days; wherein the PD-L1(+) natural killer cells express soluble IL-15. In embodiments, the disclosure provides methods of treating cancer in a patient in need thereof comprising administering to the patient an effective amount of PD-L1(+) natural killer cells about once every three days; wherein the PD-L1(+) natural killer cells express soluble IL-15. In embodiments, the disclosure provides methods of treating cancer in a patient in need thereof comprising administering to the patient an effective amount of PD-L1(+) natural killer cells about once every four days; wherein the PD-L1(+) natural killer cells express soluble IL-15. In embodiments, the disclosure provides methods of treating cancer in a patient in need thereof comprising administering to the patient an effective amount of PD-L1(+) natural killer cells about once every three or four days; wherein the PD-L1(+) natural killer cells express soluble IL-15. In embodiments, the disclosure provides methods of treating cancer in a patient in need thereof comprising administering to the patient an effective amount of PD-L1(+) natural killer cells about once every five days; wherein the PD-L1(+) natural killer cells express soluble IL-15. In embodiments, the disclosure provides methods of treating cancer in a patient in need thereof comprising administering to the patient an effective amount of PD-L1(+) natural killer cells about once every six days; wherein the PD-L1(+) natural killer cells express soluble IL-15. In embodiments, the patient is administered from about 1×10⁶ to about 1×10¹² of the PD-L1(+) natural killer cells. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells express soluble IL-15 and truncated EGFR. In embodiments, the cancer is lung cancer. In embodiments, the cancer is non-small cell lung cancer. In embodiments, the cancer is advanced non-small cell lung cancer. In embodiments, the cancer is metastatic non-small cell lung cancer. In embodiments, the cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the cancer is metastatic cancer. In embodiments, the patient is refractory to a PD-1 inhibitor and/or a PD-L1 inhibitor. In embodiments, the patient is refractory to chemotherapy.

In embodiments, the disclosure provides methods of treating non-small cell lung cancer in a patient in need thereof comprising administering to the patient an effective amount of PD-L1(+) natural killer cells once per week; wherein the PD-L1(+) natural killer cells express soluble IL-15. In embodiments, the disclosure provides methods of treating non-small cell lung cancer in a patient in need thereof comprising administering to the patient an effective amount of PD-L1(+) natural killer cells twice per week; wherein the PD-L1(+) natural killer cells express soluble IL-15. In embodiments, the PD-L1(+) natural killer cells express soluble IL-15 and truncated EGFR. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient from about 1×10⁶ to about 1×10¹² of PD-L1(+) natural killer cells. In embodiments, the disclosure provides methods of treating cancer in a patient in need thereof comprising administering to the patient from about 1×10⁷ to about 1×10¹² of PD-L1(+) natural killer cells. In embodiments, the disclosure provides methods of treating cancer in a patient in need thereof comprising administering to the patient from about 2×10⁷ to about 1×10¹¹ of PD-L1(+) natural killer cells. In embodiments, the disclosure provides methods of treating cancer in a patient in need thereof comprising administering to the patient from about 4×10⁷ to about 2×10⁹ of PD-L1(+) natural killer cells. In embodiments, the disclosure provides methods of treating cancer in a patient in need thereof comprising administering to the patient from about 1×10⁸ to about 2×10⁹ of PD-L1(+) natural killer cells. In embodiments, the PD-L1(+) natural killer cells are administered to the patient once per day. In embodiments, the PD-L1(+) natural killer cells are administered to the patient once every two days. In embodiments, the PD-L1(+) natural killer cells are administered to the patient once every three days. In embodiments, the PD-L1(+) natural killer cells are administered to the patient once every four days. In embodiments, the PD-L1(+) natural killer cells are administered to the patient once every three or four days. In embodiments, the PD-L1(+) natural killer cells are administered to the patient once every five days. In embodiments, the PD-L1(+) natural killer cells are administered to the patient once every six days. In embodiments, the PD-L1(+) natural killer cells are administered to the patient once per week. In embodiments, the PD-L1(+) natural killer cells are administered to the patient twice per week. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient from about 1×10⁶ to about 1×10¹² of PD-L1(+) natural killer cells; wherein the PD-L1(+) natural killer cells express soluble IL-15. In embodiments, the disclosure provides methods of treating cancer in a patient in need thereof comprising administering to the patient from about 1×10⁷ to about 1×10¹² of PD-L1(+) natural killer cells; wherein the PD-L1(+) natural killer cells express soluble IL-15. In embodiments, the disclosure provides methods of treating cancer in a patient in need thereof comprising administering to the patient from about 2×10⁷ to about 1×10¹¹ of PD-L1(+) natural killer cells; wherein the PD-L1(+) natural killer cells express soluble IL-15. In embodiments, the disclosure provides methods of treating cancer in a patient in need thereof comprising administering to the patient from about 4×10⁷ to about 2×10⁹ of PD-L1(+) natural killer cells; wherein the PD-L1(+) natural killer cells express soluble IL-15. In embodiments, the disclosure provides methods of treating cancer in a patient in need thereof comprising administering to the patient from about 1×10⁸ to about 2×10⁹ of PD-L1(+) natural killer cells; wherein the PD-L1(+) natural killer cells express soluble IL-15. In embodiments, the PD-L1(+) natural killer cells express soluble IL-15 and truncated EGFR. In embodiments, the PD-L1(+) natural killer cells are administered to the patient once per day. In embodiments, the PD-L1(+) natural killer cells are administered to the patient once every two days. In embodiments, the PD-L1(+) natural killer cells are administered to the patient once every three days. In embodiments, the PD-L1(+) natural killer cells are administered to the patient once every four days. In embodiments, the PD-L1(+) natural killer cells are administered to the patient once every three or four days. In embodiments, the PD-L1(+) natural killer cells are administered to the patient once every five days. In embodiments, the PD-L1(+) natural killer cells are administered to the patient once every six days. In embodiments, the PD-L1(+) natural killer cells are administered to the patient once per week. In embodiments, the PD-L1(+) natural killer cells are administered to the patient twice per week. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy.

In embodiments, the disclosure provides methods of treating non-small cell lung cancer in a patient in need thereof comprising administering to the patient from about 1×10⁶ to about 1×10¹² of PD-L1(+) natural killer cells; wherein the PD-L1(+) natural killer cells express soluble IL-15. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells express soluble IL-15 and truncated EGFR. In embodiments, the PD-L1(+) natural killer cells are administered to the patient once per day. In embodiments, the PD-L1(+) natural killer cells are administered to the patient once every two days. In embodiments, the PD-L1(+) natural killer cells are administered to the patient once every three days. In embodiments, the PD-L1(+) natural killer cells are administered to the patient once every four days. In embodiments, the PD-L1(+) natural killer cells are administered to the patient once every three or four days. In embodiments, the PD-L1(+) natural killer cells are administered to the patient once every five days. In embodiments, the PD-L1(+) natural killer cells are administered to the patient once every six days. In embodiments, the PD-L1(+) natural killer cells are administered to the patient once per week. In embodiments, the PD-L1(+) natural killer cells are administered to the patient twice per week. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient from about 1×10⁶ to about 1×10¹² of PD-L1(+) natural killer cells once per week. In embodiments, the disclosure provides methods of treating cancer in a patient in need thereof comprising administering to the patient from about 1×10⁶ to about 1×10¹² of PD-L1(+) natural killer cells twice per week. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient from about 1×10⁶ to about 1×10¹² of PD-L1(+) natural killer cells once per week; wherein the PD-L1(+) natural killer cells express soluble IL-15. In embodiments, the disclosure provides methods of treating cancer in a patient in need thereof comprising administering to the patient from about 1×10⁶ to about 1×10¹² of PD-L1(+) natural killer cells twice per week; wherein the PD-L1(+) natural killer cells express soluble IL-15. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells express soluble IL-15 and truncated EGFR. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy.

In embodiments, the disclosure provides methods of treating non-small cell lung cancer in a patient in need thereof comprising administering to the patient from about 1×10⁶ to about 1×10¹² of PD-L1(+) natural killer cells once per week; wherein the PD-L1(+) natural killer cells express soluble IL-15. In embodiments, the disclosure provides methods of treating non-small cell lung cancer in a patient in need thereof comprising administering to the patient from about 1×10⁶ to about 1×10¹² of PD-L1(+) natural killer cells twice per week; wherein the PD-L1(+) natural killer cells express soluble IL-15. In embodiments, the PD-L1(+) natural killer cells express soluble IL-15 and truncated EGFR. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient an effective amount of PD-L1(+) natural killer cells and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells and an effective amount of a PD-L1 inhibitor. In embodiments, the disclosure provides methods of treating cancer in a patient in need thereof comprising administering to the patient an effective amount of PD-L1(+) natural killer cells and an effective amount of a PD-1 inhibitor. In embodiments, the disclosure provides methods of treating cancer in a patient in need thereof comprising administering to the patient an effective amount of PD-L1(+) natural killer cells about once per week, and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about twice per week and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once per day and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every two days and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every three days and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every four days and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every three or four days and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every five days and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every six days and an effective amount of a PD-L1 inhibitor or a PD-1 inhibitor. In embodiments, the PD-L1(+) natural killer cells express soluble IL-15 and truncated EGFR. In embodiments, the patient is administered a PD-L1 inhibitor. In embodiments, the PD-L1 inhibitor is atezolizumab, avelumab, durvalumab, KN035, CK-301, AUNP12, CA-170, or BMS-986189. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab. In embodiments, the patient is administered a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the patient is administered from about 1×10⁶ to about 1×10¹² of the PD-L1(+) natural killer cells. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR. In embodiments, the cancer is lung cancer. In embodiments, the cancer is non-small cell lung cancer. In embodiments, the cancer is advanced non-small cell lung cancer. In embodiments, the cancer is metastatic non-small cell lung cancer. In embodiments, the cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the cancer is metastatic cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient an effective amount of PD-L1(+) natural killer cells about once per week; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about twice per week; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once per day; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every two days; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every three days; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every four days; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every three or four days; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every five days; wherein the PD-L1(+) natural killer cells express soluble IL-15; and an effective amount of a PD-L1 inhibitor or a PD-1 inhibitor. In embodiments, the disclosure provides methods of treating cancer in a patient in need thereof comprising administering to the patient an effective amount of PD-L1(+) natural killer cells about once every six days; wherein the PD-L1(+) natural killer cells express soluble IL-15; and an effective amount of a PD-L1 inhibitor or a PD-1 inhibitor. In embodiments, the PD-L1(+) natural killer cells express soluble IL-15 and truncated EGFR. In embodiments, the patient is administered a PD-L1 inhibitor. In embodiments, the PD-L1 inhibitor is atezolizumab, avelumab, durvalumab, KN035, CK-301, AUNP12, CA-170, or BMS-986189. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab. In embodiments, the patient is administered a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the patient is administered from about 1×10⁶ to about 1×10¹² of the PD-L1(+) natural killer cells. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the cancer is lung cancer. In embodiments, the cancer is non-small cell lung cancer. In embodiments, the cancer is advanced non-small cell lung cancer. In embodiments, the cancer is metastatic non-small cell lung cancer. In embodiments, the cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the cancer is metastatic cancer. In embodiments, the patient is refractory to a PD-1 inhibitor and/or a PD-L1 inhibitor. In embodiments, the patient is refractory to chemotherapy.

In embodiments, the disclosure provides methods of treating non-small cell lung cancer in a patient in need thereof comprising administering to the patient an effective amount of PD-L1(+) natural killer cells once per week; wherein the PD-L1(+) natural killer cells express soluble IL-15; and an effective amount of a PD-L1 inhibitor or a PD-1 inhibitor. In embodiments, the PD-L1(+) natural killer cells express soluble IL-15 and truncated EGFR. In embodiments, the patient is administered a PD-L1 inhibitor. In embodiments, the PD-L1 inhibitor is atezolizumab, avelumab, durvalumab, KN035, CK-301, AUNP12, CA-170, or BMS-986189. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab. In embodiments, the patient is administered a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the disclosure provides methods of treating non-small cell lung cancer in a patient in need thereof comprising administering to the patient an effective amount of PD-L1(+) natural killer cells twice per week; wherein the PD-L1(+) natural killer cells express soluble IL-15. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient from about 1×10⁶ to about 1×10¹² of PD-L1(+) natural killer cells and an effective amount of a PD-L1 inhibitor or a PD-1 inhibitor. In embodiments, the disclosure provides methods of treating cancer in a patient in need thereof comprising administering to the patient from about 1×10⁷ to about 1×10¹² of PD-L1(+) natural killer cells and an effective amount of a PD-L1 inhibitor or a PD-1 inhibitor. In embodiments, the disclosure provides methods of treating cancer in a patient in need thereof comprising administering to the patient from about 2×10⁷ to about 1×10¹⁰ of PD-L1(+) natural killer cells and an effective amount of a PD-L1 inhibitor or a PD-1 inhibitor. In embodiments, the disclosure provides methods of treating cancer in a patient in need thereof comprising administering to the patient from about 4×10⁷ to about 2×10⁹ of PD-L1(+) natural killer cells and an effective amount of a PD-L1 inhibitor or a PD-1 inhibitor. In embodiments, the disclosure provides methods of treating cancer in a patient in need thereof comprising administering to the patient from about 1×10⁸ to about 2×10⁹ of PD-L1(+) natural killer cells and an effective amount of a PD-L1 inhibitor or a PD-1 inhibitor. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR. In embodiments, the patient is administered a PD-L1 inhibitor. In embodiments, the PD-L1 inhibitor is atezolizumab, avelumab, durvalumab, KN035, CK-301, AUNP12, CA-170, or BMS-986189. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab. In embodiments, the patient is administered a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the PD-L1(+) natural killer cells are administered to the patient once per day. In embodiments, the PD-L1(+) natural killer cells are administered to the patient once every two days. In embodiments, the PD-L1(+) natural killer cells are administered to the patient once every three days. In embodiments, the PD-L1(+) natural killer cells are administered to the patient once every four days. In embodiments, the PD-L1(+) natural killer cells are administered to the patient once every three or four days. In embodiments, the PD-L1(+) natural killer cells are administered to the patient once every five days. In embodiments, the PD-L1(+) natural killer cells are administered to the patient once every six days. In embodiments, the PD-L1(+) natural killer cells are administered to the patient once per week. In embodiments, the PD-L1(+) natural killer cells are administered to the patient twice per week. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy.

In embodiments, the disclosure provides methods of treating non-small cell lung cancer in a patient in need thereof comprising administering to the patient from about 1×10⁶ to about 1×10¹¹ of PD-L1(+) natural killer cells; wherein the PD-L1(+) natural killer cells express soluble IL-15; and an effective amount of a PD-L1 inhibitor or a PD-1 inhibitor. In embodiments, the PD-L1(+) natural killer cells express soluble IL-15 and truncated EGFR. In embodiments, the patient is administered a PD-L1 inhibitor. In embodiments, the PD-L1 inhibitor is atezolizumab, avelumab, durvalumab, KN035, CK-301, AUNP12, CA-170, or BMS-986189. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab. In embodiments, the patient is administered a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells are administered to the patient once per day. In embodiments, the PD-L1(+) natural killer cells are administered to the patient once every two days. In embodiments, the PD-L1(+) natural killer cells are administered to the patient once every three days. In embodiments, the PD-L1(+) natural killer cells are administered to the patient once every four days. In embodiments, the PD-L1(+) natural killer cells are administered to the patient once every three or four days. In embodiments, the PD-L1(+) natural killer cells are administered to the patient once every five days. In embodiments, the PD-L1(+) natural killer cells are administered to the patient once every six days. In embodiments, the PD-L1(+) natural killer cells are administered to the patient once per week. In embodiments, the PD-L1(+) natural killer cells are administered to the patient twice per week. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the patient is refractory to a PD-1 inhibitor and/or a PD-L1 inhibitor. In embodiments, the patient is refractory to chemotherapy.

In embodiments, the disclosure provides methods of treating cancer in a patient in need thereof comprising administering to the patient from about 1×10⁶ to about 1×10¹² of PD-L1(+) natural killer cells once per week and an effective amount of a PD-L1 inhibitor or a PD-1 inhibitor. In embodiments, the disclosure provides methods of treating cancer in a patient in need thereof comprising administering to the patient from about 1×10⁶ to about 1×10¹² of PD-L1(+) natural killer cells twice per week and an effective amount of a PD-L1 inhibitor or a PD-1 inhibitor. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR. In embodiments, the patient is administered a PD-L1 inhibitor. In embodiments, the PD-L1 inhibitor is atezolizumab, avelumab, durvalumab, KN035, CK-301, AUNP12, CA-170, or BMS-986189. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab. In embodiments, the patient is administered a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the patient is administered from about 1×10⁷ to about 1×10¹² of the PD-L1(+) natural killer cells. In embodiments, the patient is administered from about 2×10⁷ to about 1×10¹⁰ of the PD-L1(+) natural killer cells. In embodiments, the patient is administered from about 4×10⁷ to about 2×10⁹ of the PD-L1(+) natural killer cells. In embodiments, the patient is administered from about 1×10⁸ to about 2×10⁹ of the PD-L1(+) natural killer cells. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy.

In embodiments, the disclosure provides methods of treating non-small cell lung cancer in a patient in need thereof comprising administering to the patient from about 1×10⁶ to about 1×10¹² of PD-L1(+) natural killer cells once per week; wherein the PD-L1(+) natural killer cells express soluble IL-15; and an effective amount of a PD-L1 inhibitor or a PD-1 inhibitor. In embodiments, the disclosure provides methods of treating non-small cell lung cancer in a patient in need thereof comprising administering to the patient from about 1×10⁶ to about 1×10¹² of PD-L1(+) natural killer cells twice per week; wherein the PD-L1(+) natural killer cells express soluble IL-15; and an effective amount of a PD-L1 inhibitor or a PD-1 inhibitor. In embodiments, the PD-L1(+) natural killer cells express soluble IL-15 and truncated EGFR. In embodiments, the patient is administered a PD-L1 inhibitor. In embodiments, the PD-L1 inhibitor is atezolizumab, avelumab, durvalumab, KN035, CK-301, AUNP12, CA-170, or BMS-986189. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab. In embodiments, the patient is administered a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the disclosure provides methods of treating non-small cell lung cancer in a patient in need thereof is provided, the method including administering to the patient: (i) from about 1×10⁶ to about 1×10¹² of PD-L1(+) natural killer cells once per week; wherein the PD-L1(+) natural killer cells express soluble IL-15; and (ii) an effective amount of atezolizumab. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the patient is refractory to a PD-1 inhibitor and/or a PD-L1 inhibitor. In embodiments, the patient is refractory to chemotherapy.

In embodiments, the disclosure provides methods of treating cancer by administering to a patient: (i) an effective amount of PD-L1(+) natural killer cells by one infusion per week for a total of four infusions; (ii) an increased number of an effective amount of PD-L1(+) natural killer cells infusions (from four to eight), administered weekly over an eight week time period, or (iii) an increased number of an effective amount of PD-L1(+) natural killer cells infusions (from four to eight), with two infusions administered weekly over a four week period. In embodiments, the non-small cell lung cancer is advanced non-small cell lung cancer. In embodiments, the non-small cell lung cancer is metastatic non-small cell lung cancer. In embodiments, the non-small cell lung cancer is recurrent non-small cell lung cancer. In embodiments, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR.

In embodiments, the disclosure provides methods of treating non-small cell lung cancer by administering to a patient: (i) an effective amount of PD-L1(+) natural killer cells by one infusion per week for a total of four infusions; (ii) an increased number of an effective amount of PD-L1(+) natural killer cells infusions (from four to eight), administered weekly over an eight week time period, or (iii) an increased number of an effective amount of PD-L1(+) natural killer cells infusions (from four to eight), with two infusions administered weekly over a four week period. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR.

In embodiments, the methods described herein comprise administering to the patient from about 1×10⁶ to about 1×10¹² of PD-L1(+) natural killer cells. In embodiments, the method includes administering to the patient from about 2×10⁶ to about 1×10¹² of PD-L1(+) natural killer cells. In embodiments, the method includes administering to the patient from about 3×10⁶ to about 1×10¹² of PD-L1(+) natural killer cells. In embodiments, the method includes administering to the patient from about 4×10⁶ to about 1×10¹² of PD-L1(+) natural killer cells. In embodiments, the method includes administering to the patient from about 5×10⁶ to about 1×10¹² of PD-L1(+) natural killer cells. In embodiments, the method includes administering to the patient from about 6×10⁶ to about 1×10¹² of PD-L1(+) natural killer cells. In embodiments, the method includes administering to the patient from about 7×10⁶ to about 1×10¹² of PD-L1(+) natural killer cells. In embodiments, the method includes administering to the patient from about 8×10⁶ to about 1×10¹² of PD-L1(+) natural killer cells. In embodiments, the method includes administering to the patient from about 9×10⁶ to about 1×10¹² of PD-L1(+) natural killer cells. In embodiments, the method includes administering to the patient from about 1×10⁷ to about 1×10¹² of PD-L1(+) natural killer cells. In embodiments, the method includes administering to the patient from about 2×10⁷ to about 1×10¹² of PD-L1(+) natural killer cells. In embodiments, the method includes administering to the patient from about 3×10⁷ to about 1×10¹² of PD-L1(+) natural killer cells. In embodiments, the method includes administering to the patient from about 4×10⁷ to about 1×10¹² of PD-L1(+) natural killer cells. In embodiments, the method includes administering to the patient from about 5×10⁷ to about 1×10¹² of PD-L1(+) natural killer cells. In embodiments, the method includes administering to the patient from about 6×10⁷ to about 1×10¹² of PD-L1(+) natural killer cells. In embodiments, the method includes administering to the patient from about 7×10⁷ to about 1×10¹² of PD-L1(+) natural killer cells. In embodiments, the method includes administering to the patient from about 8×10⁷ to about 1×10¹² of PD-L1(+) natural killer cells. In embodiments, the method includes administering to the patient from about 9×10⁷ to about 1×10¹² of PD-L1(+) natural killer cells. In embodiments, the method includes administering to the patient from about 1×10⁸ to about 1×10¹² of PD-L1(+) natural killer cells. In embodiments, the method includes administering to the patient from about 2×10⁸ to about 1×10¹² of PD-L1(+) natural killer cells. In embodiments, the method includes administering to the patient from about 3×10¹ to about 1×10¹² of PD-L1(+) natural killer cells. In embodiments, the method includes administering to the patient from about 4×10⁸ to about 1×10¹² of PD-L1(+) natural killer cells. In embodiments, the method includes administering to the patient from about 5×10⁸ to about 1×10¹² of PD-L1(+) natural killer cells. In embodiments, the method includes administering to the patient from about 6×10⁸ to about 1×10¹² of PD-L1(+) natural killer cells. In embodiments, the method includes administering to the patient from about 7×10⁸ to about 1×10¹² of PD-L1(+) natural killer cells. In embodiments, the method includes administering to the patient from about 8×10⁸ to about 1×10¹² of PD-L1(+) natural killer cells. In embodiments, the method includes administering to the patient from about 9×10⁸ to about 1×10¹² of PD-L1(+) natural killer cells. In embodiments, the method includes administering to the patient from about 1×10⁹ to about 1×10¹² of PD-L1(+) natural killer cells. In embodiments, the method includes administering to the patient from about 2×10⁹ to about 1×10¹² of PD-L1(+) natural killer cells. In embodiments, the method includes administering to the patient from about 3×10⁹ to about 1×10¹² of PD-L1(+) natural killer cells. In embodiments, the method includes administering to the patient from about 4×10⁹ to about 1×10¹² of PD-L1(+) natural killer cells. In embodiments, the method includes administering to the patient from about 5×10⁹ to about 1×10¹² of PD-L1(+) natural killer cells. In embodiments, the method includes administering to the patient from about 6×10⁹ to about 1×10¹² of PD-L1(+) natural killer cells. In embodiments, the method includes administering to the patient from about 7×10⁹ to about 1×10¹² of PD-L1(+) natural killer cells. In embodiments, the method includes administering to the patient from about 8×10⁹ to about 1×10¹² of PD-L1(+) natural killer cells. In embodiments, the method includes administering to the patient from about 9×10⁹ to about 1×10¹² of PD-L1(+) natural killer cells. In embodiments, the method includes administering to the patient from about 1×10¹⁰ to about 1×10¹² of PD-L1(+) natural killer cells. In embodiments, the method includes administering to the patient from about 2×10¹⁰ to about 1×10¹² of PD-L1(+) natural killer cells. In embodiments, the method includes administering to the patient from about 3×10¹⁰ to about 1×10¹² of PD-L1(+) natural killer cells. In embodiments, the method includes administering to the patient from about 4×10¹⁰ to about 1×10¹² of PD-L1(+) natural killer cells. In embodiments, the method includes administering to the patient from about 5×10¹⁰ to about 1×10¹² of PD-L1(+) natural killer cells. In embodiments, the method includes administering to the patient from about 6×10¹⁰ to about 1×10¹² of PD-L1(+) natural killer cells. In embodiments, the method includes administering to the patient from about 7×10¹⁰ to about 1×10¹² of PD-L1(+) natural killer cells. In embodiments, the method includes administering to the patient from about 8×10¹⁰ to about 1×10¹² of PD-L1(+) natural killer cells. In embodiments, the method includes administering to the patient from about 9×10¹⁰ to about 1×10¹² of PD-L1(+) natural killer cells. In embodiments, the method includes administering to the patient from about 1×10¹¹ to about 1×10¹² of PD-L1(+) natural killer cells. In embodiments, the method includes administering to the patient from about 2×10¹¹ to about 1×10¹² of PD-L1(+) natural killer cells. In embodiments, the method includes administering to the patient from about 3×10¹¹ to about 1×10¹² of PD-L1(+) natural killer cells. In embodiments, the method includes administering to the patient from about 4×10¹¹ to about 1×10¹² of PD-L1(+) natural killer cells. In embodiments, the method includes administering to the patient from about 5×10¹¹ to about 1×10¹² of PD-L1(+) natural killer cells. In embodiments, the method includes administering to the patient from about 6×10¹¹ to about 1×10¹² of PD-L1(+) natural killer cells. In embodiments, the method includes administering to the patient from about 7×10¹¹ to about 1×10¹² of PD-L1(+) natural killer cells. In embodiments, the method includes administering to the patient from about 8×10¹¹ to about 1×10¹² of PD-L1(+) natural killer cells. In embodiments, the method includes administering to the patient from about 9×10¹¹ to about 1×10¹² of PD-L1(+) natural killer cells. As described herein, the PD-L1(+) natural killer cells express soluble IL-15. In embodiments, the PD-L1(+) natural killer cells express soluble IL-15 and truncated EGFR.

In embodiments of the methods described herein, the methods include administering to the patient from about 1×10⁶ to about 9×10¹¹ of PD-L1(+) natural killer cells. In embodiments, the method includes administering to the patient from about 1×10⁶ to about 8×10¹¹ of PD-L1(+) natural killer cells. In embodiments, the method includes administering to the patient from about 1×10⁶ to about 7×10¹¹ of PD-L1(+) natural killer cells. In embodiments, the method includes administering to the patient from about 1×10⁶ to about 6×10¹¹ of PD-L1(+) natural killer cells. In embodiments, the method includes administering to the patient from about 1×10⁶ to about 5×10¹¹ of PD-L1(+) natural killer cells. In embodiments, the method includes administering to the patient from about 1×10⁶ to about 4×10¹¹ of PD-L1(+) natural killer cells. In embodiments, the method includes administering to the patient from about 1×10⁶ to about 3×10¹¹ of PD-L1(+) natural killer cells. In embodiments, the method includes administering to the patient from about 1×10⁶ to about 2×10¹¹ of PD-L1(+) natural killer cells. In embodiments, the method includes administering to the patient from about 1×10⁶ to about 1×10¹¹ of PD-L1(+) natural killer cells. In embodiments, the method includes administering to the patient from about 1×10⁶ to about 9×10¹⁰ of PD-L1(+) natural killer cells. In embodiments, the method includes administering to the patient from about 1×10⁶ to about 8×10¹⁰ of PD-L1(+) natural killer cells. In embodiments, the method includes administering to the patient from about 1×10⁶ to about 7×10¹⁰ of PD-L1(+) natural killer cells. In embodiments, the method includes administering to the patient from about 1×10⁶ to about 6×10¹⁰ of PD-L1(+) natural killer cells. In embodiments, the method includes administering to the patient from about 1×10⁶ to about 5×10¹⁰ of PD-L1(+) natural killer cells. In embodiments, the method includes administering to the patient from about 1×10⁶ to about 4×10¹⁰ of PD-L1(+) natural killer cells. In embodiments, the method includes administering to the patient from about 1×10⁶ to about 3×10¹⁰ of PD-L1(+) natural killer cells. In embodiments, the method includes administering to the patient from about 1×10⁶ to about 2×10¹⁰ of PD-L1(+) natural killer cells. In embodiments, the method includes administering to the patient from about 1×10⁶ to about 1×10¹⁰ of PD-L1(+) natural killer cells. In embodiments, the method includes administering to the patient from about 1×10⁶ to about 9×10⁹ of PD-L1(+) natural killer cells. In embodiments, the method includes administering to the patient from about 1×10⁶ to about 8×10⁹ of PD-L1(+) natural killer cells. In embodiments, the method includes administering to the patient from about 1×10⁶ to about 7×10⁹ of PD-L1(+) natural killer cells. In embodiments, the method includes administering to the patient from about 1×10⁶ to about 6×10⁹ of PD-L1(+) natural killer cells. In embodiments, the method includes administering to the patient from about 1×10⁶ to about 5×10⁹ of PD-L1(+) natural killer cells. In embodiments, the method includes administering to the patient from about 1×10⁶ to about 4×10⁹ of PD-L1(+) natural killer cells. In embodiments, the method includes administering to the patient from about 1×10⁶ to about 3×10⁹ of PD-L1(+) natural killer cells. In embodiments, the method includes administering to the patient from about 1×10⁶ to about 2×10⁹ of PD-L1(+) natural killer cells. In embodiments, the method includes administering to the patient from about 1×10⁶ to about 1×10⁹ of PD-L1(+) natural killer cells. In embodiments, the method includes administering to the patient from about 1×10⁶ to about 9×10⁸ of PD-L1(+) natural killer cells. In embodiments, the method includes administering to the patient from about 1×10⁶ to about 8×10⁹ of PD-L1(+) natural killer cells. In embodiments, the method includes administering to the patient from about 1×10⁶ to about 7×10⁸ of PD-L1(+) natural killer cells. In embodiments, the method includes administering to the patient from about 1×10⁶ to about 6×10⁸ of PD-L1(+) natural killer cells. In embodiments, the method includes administering to the patient from about 1×10⁶ to about 5×10⁸ of PD-L1(+) natural killer cells. In embodiments, the method includes administering to the patient from about 1×10⁶ to about 4×10⁸ of PD-L1(+) natural killer cells. In embodiments, the method includes administering to the patient from about 1×10⁶ to about 3×10⁸ of PD-L1(+) natural killer cells. In embodiments, the method includes administering to the patient from about 1×10⁶ to about 2×10⁸ of PD-L1(+) natural killer cells. In embodiments, the method includes administering to the patient from about 1×10⁶ to about 1×10⁸ of PD-L1(+) natural killer cells. In embodiments, the method includes administering to the patient from about 1×10⁶ to about 9×10⁷ of PD-L1(+) natural killer cells. In embodiments, the method includes administering to the patient from about 1×10⁶ to about 8×10⁷ of PD-L1(+) natural killer cells. In embodiments, the method includes administering to the patient from about 1×10⁶ to about 7×10⁷ of PD-L1(+) natural killer cells. In embodiments, the method includes administering to the patient from about 1×10⁶ to about 6×10⁷ of PD-L1(+) natural killer cells. In embodiments, the method includes administering to the patient from about 1×10⁶ to about 5×10⁷ of PD-L1(+) natural killer cells. In embodiments, the method includes administering to the patient from about 1×10⁶ to about 4×10⁷ of PD-L1(+) natural killer cells. In embodiments, the method includes administering to the patient from about 1×10⁶ to about 3×10⁷ of PD-L1(+) natural killer cells. In embodiments, the method includes administering to the patient from about 1×10⁶ to about 2×10⁷ of PD-L1(+) natural killer cells. In embodiments, the method includes administering to the patient from about 1×10⁶ to about 1×10⁷ of PD-L1(+) natural killer cells. In embodiments, the method includes administering to the patient from about 1×10⁶ to about 9×10⁶ of PD-L1(+) natural killer cells. In embodiments, the method includes administering to the patient from about 1×10⁶ to about 8×10⁶ of PD-L1(+) natural killer cells. In embodiments, the method includes administering to the patient from about 1×10⁶ to about 7×10⁶ of PD-L1(+) natural killer cells. In embodiments, the method includes administering to the patient from about 1×10⁶ to about 6×10⁶ of PD-L1(+) natural killer cells. In embodiments, the method includes administering to the patient from about 1×10⁶ to about 5×10⁶ of PD-L1(+) natural killer cells. In embodiments, the method includes administering to the patient from about 1×10⁶ to about 4×10⁶ of PD-L1(+) natural killer cells. In embodiments, the method includes administering to the patient from about 1×10⁶ to about 3×10⁶ of PD-L1(+) natural killer cells. In embodiments, the method includes administering to the patient from about 1×10⁶ to about 2×10⁶ of PD-L1(+) natural killer cells. In embodiments, the method includes administering to the patient about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, or 1×10¹² of PD-L1(+) natural killer cells. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR.

In embodiments of the methods described herein, the methods include administering from about 1×10⁷ to about 1×10¹² of the PD-L1(+) natural killer cells. In embodiments, the method includes administering from about 2×10⁷ to about 1×10¹⁰ of the PD-L1(+) natural killer cells. In embodiments, the method includes administering from about 4×10⁷ to about 2×10⁹ of the PD-L1(+) natural killer cells. In embodiments, the method includes administering from about 1×10⁸ to about 2×10⁹ of the PD-L1(+) natural killer cells. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR.

In embodiments of the methods described herein, the methods include administering about 5×10⁷ of the PD-L1(+) natural killer cells. In embodiments, the method includes administering about 1×10⁸ of the PD-L1(+) natural killer cells. In embodiments, the method includes administering about 2×10⁸ of the PD-L1(+) natural killer cells. In embodiments, the method includes administering about 4×10⁸ of the PD-L1(+) natural killer cells. In embodiments, the method includes administering about 5×10⁸ of the PD-L1(+) natural killer cells. In embodiments, the method includes administering 1×10⁹ of the PD-L1(+) natural killer cells. In embodiments, the PD-L1(+) natural killer cells express soluble IL-15. In embodiments, the PD-L1(+) natural killer cells express truncated EGFR. In embodiments, the PD-L1(+) natural killer cells express soluble IL-15 and truncated EGFR.

In embodiments, the effective amount of PD-L1(+) natural killer cells are administered to the patient once per week for four weeks. In embodiments, administration of the PD-L1(+) natural killer cells is discontinued for 4 weeks; wherein another 8 week treatment cycle is optionally initiated one or more times thereafter. For example, an 8 week treatment cycle includes 4 weeks of treatment followed by 4 weeks without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered within the 8 week treatment cycle, which is repeated as necessary.

In embodiments, the effective amount of PD-L1(+) natural killer cells are administered to the patient once per week for five weeks. In embodiments, administration of the PD-L1(+) natural killer cells is discontinued for 3 weeks; wherein another 8 week treatment cycle is optionally initiated one or more times thereafter. For example, an 8 week treatment cycle includes 5 weeks of treatment followed by 3 weeks without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient once per week for six weeks. In embodiments, administration of the PD-L1(+) natural killer cells is discontinued for 2 weeks; wherein another 8 week treatment cycle is optionally initiated one or more times thereafter. For example, an 8 week treatment cycle includes 6 weeks of treatment followed by 2 weeks without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient once per week for seven weeks. In embodiments, administration of the PD-L1(+) natural killer cells is discontinued for 1 week; wherein another 8 week treatment cycle is optionally initiated one or more times thereafter. For example, an 8 week treatment cycle includes 7 weeks of treatment followed by 1 week without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient once per week for eight weeks. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient twice per week for four weeks. In embodiments, administration of the PD-L1(+) natural killer cells is discontinued for 4 weeks; wherein another 8 week treatment cycle is optionally initiated one or more times thereafter. For example, an 8 week treatment cycle includes 4 weeks of treatment followed by 4 weeks without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient twice per week for five weeks. In embodiments, administration of the PD-L1(+) natural killer cells is discontinued for 3 weeks; wherein another 8 week treatment cycle is optionally initiated one or more times thereafter. For example, an 8 week treatment cycle includes 5 weeks of treatment followed by 3 weeks without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient twice per week for six weeks. In embodiments, wherein administration of the PD-L1(+) natural killer cells is discontinued for 2 weeks; wherein another 8 week treatment cycle is optionally initiated one or more times thereafter. For example, an 8 week treatment cycle includes 6 weeks of treatment followed by 2 weeks without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient twice per week for seven weeks. In embodiments, administration of the PD-L1(+) natural killer cells is discontinued for 1 week; wherein another 8 week treatment cycle is optionally initiated one or more times thereafter. For example, an 8 week treatment cycle includes 7 weeks of treatment followed by 1 week without treatment. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

In embodiments, the effective amount of PD-L1(+) natural killer cells are administered to the patient twice per week for eight weeks. This 8 week treatment cycle can repeated once, twice, three times, or more. In embodiments, the 8 week treatment cycle is repeated 9 times for a total period of time of 18 months. In embodiments, the 8 week treatment cycle is repeated 12 times for a total period of time of 24 months. In embodiments, the method further comprises administering a checkpoint inhibitor to the patient. In embodiments, the checkpoint inhibitor is a PD-1 inhibitor. In embodiments, the checkpoint inhibitor is a PD-L1 inhibitor. In embodiments, the PD-L1 inhibitor is atezolizumab. In embodiments, the methods further comprise administering to the patient about 840 mg of atezolizumab about once every 14 days. In embodiments, the methods further comprise administering to the patient about 120 mg of atezolizumab about once every 21 days. In embodiments, the methods further comprise administering to the patient about 1680 mg of atezolizumab about once every 28 days. In embodiments, the checkpoint inhibitor, such as atezolizumab, is administered with the 8 week treatment cycle, which is repeated as necessary.

For the methods provided herein, in embodiments, the PD-L1(+) natural killer cells are administered intravenously as an infusion for about 30 minutes to about 120 minutes. In embodiments, the PD-L1(+) natural killer cells are administered intravenously as an infusion for about 60 minutes to about 90 minutes. In embodiments, the PD-L1(+) natural killer cells are administered intravenously as an infusion for about 30 minutes to about 60 minutes. In embodiments, the PD-L1(+) natural killer cells are administered intravenously as an infusion for about 60 minutes to about 120 minutes. In embodiments, the PD-L1(+) natural killer cells are administered intravenously as an infusion for about 90 minutes to about 120 minutes. In embodiments, the PD-L1(+) natural killer cells are administered intravenously as an infusion for about 60 minutes.

In embodiments, the method provided herein including embodiments thereof further includes pretreating the patient with a low-dose and/or escalating dose of the PD-L1(+) natural killer cells prior to beginning treatment with the effective dose, wherein the low-dose and/or escalating dose is an amount lower than the effective dose. In embodiments, the method provided herein including embodiments thereof further includes pretreating the patient with a low-dose of the PD-L1(+) natural killer cells prior to beginning treatment with the effective dose, wherein the low-dose escalating dose is an amount lower than the effective dose. In embodiments, the method provided herein including embodiments thereof further includes pretreating the patient with an escalating dose of the PD-L1(+) natural killer cells prior to beginning treatment with the effective dose, wherein the escalating dose is an amount lower than the effective dose.

In embodiments, the method further includes administering to the patient an effective amount of a checkpoint inhibitor. In embodiments, the checkpoint inhibitor is a PD-1 inhibitor. In embodiments, the PD-1 inhibitor is pembrolizumab, nivolumab, or a combination thereof. In embodiments, the PD-1 inhibitor is pembrolizumab. In embodiments, the PD-1 inhibitor is nivolumab. In embodiments, the PD-L1 inhibitor is atezolizumab, avelumab, or durvalumab. In embodiments, the PD-L1 inhibitor is atezolizumab. In embodiments, the PD-L1 inhibitor is avelumab. In embodiments, the PD-L1 inhibitor is durvalumab.

In embodiments, the PD-L1 inhibitor is atezolizumab. In embodiments, the method includes administering to the patient about 840 mg of atezolizumab about once every 14 days. In embodiments, the method includes administering to the patient about 120 mg of atezolizumab about once every 21 days. In embodiments, the method includes administering to the patient about 1680 mg of atezolizumab about once every 28 days. In embodiments, the atezolizumab is administered to the patient by intravenous infusion. In embodiments, the atezolizumab is administered to the patient by intravenous infusion over a period of time from about 30 minutes to about 90 minutes. In embodiments, the atezolizumab is administered to the patient by intravenous infusion over a period of time of about 60 minutes.

In embodiments, the methods described herein are for treating cancer. In embodiments, the cancer is lung cancer. In embodiments, the cancer is non-small cell lung cancer. In embodiments, the non-small cell lung cancer is metastatic non-small cell lung cancer. In embodiments, the non-small cell lung cancer is recurrent non-small cell lung cancer. In embodiments, the non-small cell lung cancer is advanced non-small cell lung cancer. In embodiments, the non-small cell lung cancer is Stage IV. In embodiments, the non-small cell lung cancer is Stage III. In embodiments, the non-small cell lung cancer is Stage II. In embodiments, the non-small cell lung cancer is Stage I. In embodiments, the non-small cell lung cancer includes PD-L1(−) tumor cells. In embodiments, the non-small cell lung cancer includes PD-L1(+) tumor cells.

In embodiments of the methods described herein, the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer. In embodiments, the cancer is lung cancer. In embodiments, the cancer is neuroblastoma. In embodiments, the cancer is glioma. In embodiments, the cancer is myelodysplastic syndrome. In embodiments, the cancer is liver cancer. In embodiments, the cancer is prostate cancer. In embodiments, the cancer is pancreatic cancer. In embodiments, the cancer is gastric cancer. In embodiments, the cancer is head and neck cancer. In embodiments, the cancer is multiple myeloma. In embodiments, the cancer is biliary tract cancer. In embodiments, the cancer is ovarian cancer. In embodiments, the cancer is melanoma. In embodiments, the cancer is colorectal cancer. In embodiments, the cancer is lymphoma. In embodiments, the cancer is B cell lymphoma. In embodiments, the cancer is non-Hodgkin lymphoma. In embodiments, the cancer is leukemia. In embodiments, the cancer is acute myeloid leukemia. In embodiments, the cancer is chronic myeloid leukemia. In embodiments, the cancer is lymphoblastic leukemia. In embodiments, the patient is refractory to a PD-1 inhibitor and/or a PD-L1 inhibitor. In embodiments, the patient is refractory to chemotherapy.

In embodiments, the cancer is a neoplasm or malignant tumor. In embodiments, the cancer is a leukemia, lymphoma, carcinoma or sarcoma. In embodiments, the cancer is brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, thyroid cancer, breast cancer, cervical cancer, head & neck cancer, liver cancer, kidney cancer, lung cancer, ovarian cancer, uterine cancer, Hodgkin's Disease, or Non-Hodgkin's Lymphoma. In embodiments, the lung cancer is lung adenocarcinoma, lung squamous cell carcinoma, or non-small cell lung carcinoma. In embodiments, the cancer is leukemia. In embodiments, the cancer or leukemia is acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia. In embodiments, the cancer is acute myeloid leukemia. In embodiments, the cancer includes PDL1-negative tumor cells. In embodiments, the cancer includes PDL1-positive tumor cells.

In embodiments, the patient has been treated with a PD-1 inhibitor and/or PD-L1 inhibitor prior to treatment with the PD-L1(+) natural killer cells. In embodiments, the patient has been treated with a PD-1 inhibitor prior to treatment with the PD-L1(+) natural killer cells. In embodiments, the patient has been treated with a PD-L1 inhibitor prior to treatment with the PD-L1(+) natural killer cells. In embodiments, the patient is refractory to a PD-1 inhibitor and/or a PD-L1 inhibitor. In embodiments, the patient is refractory to a PD-1 inhibitor. In embodiments, the patient is refractory to a PD-L1 inhibitor.

For the method provided herein, in embodiments, the PD-L1(+) natural killer cells are produced by a process including the steps of: (a) isolating natural killer cells from a donor subject thereby producing a population of isolated natural killer cells; (b) deriving a population of PD-L1(+) natural killer cell from the population of isolated natural killer cells; thereby producing the PD-L1(+) natural killer cells (population of PD-L1(+) natural killer cells). In embodiments, the method further includes enriching and purifying the population of PD-L1(+) natural killer cells after step (b). In embodiments, isolating includes fluorescence-activated cell sorting, magnetic bead separation, column purification, or a combination of two or more thereof. In embodiments, isolating includes fluorescence-activated cell sorting. In embodiments, isolating includes magnetic bead separation. In embodiments, isolating includes column purification. In embodiments, the donor subject is an autologous cancer patient, a healthy donor, a matched heterologous hematopoietic stem cell donor, or a partially matched heterologous hematopoietic stem cell donor. In embodiments, the donor subject is an autologous cancer patient. In embodiments, the donor subject is a healthy donor. In embodiments, the donor subject is a matched heterologous hematopoietic stem cell donor. In embodiments, the donor subject is a partially matched heterologous hematopoietic stem cell donor.

For the methods provided herein, in embodiments, deriving includes expanding the population of PD-L1(+) natural killer cells by exposing the population of isolated natural killer cells to a feeder cell. In embodiments, the feeder cell is a K562 cell; a K562 cell expressing IL-15; a K562 cell expressing IL-21; or a K562 cell expressing IL-15 and IL-21. In embodiments, the feeder cell is a K562 cell. In embodiments, the feeder cell is a K562 cell expressing IL-15. In embodiments, the feeder cell is a K562 cell expressing IL-21. In embodiments, the feeder cell is a K562 cell expressing IL-15 and IL-21.

In embodiments, deriving includes fluorescence-activated cell sorting, magnetic bead separation, column purification, or a combination of two or more thereof. In embodiments, deriving includes fluorescence-activated cell sorting. In embodiments, deriving includes magnetic bead separation. In embodiments, deriving includes column purification.

In embodiments, deriving includes exposing the population of isolated natural killer cells to a natural killer activating agent to induce PD-L1 expression. In embodiments, the natural killer cell-activating agent is a cytokine. In embodiments, the cytokine is IL-2, IL-12, IL-15, IL-18, or a combination of two or more thereof. In embodiments, the cytokine is IL-2. In embodiments, the cytokine is IL-12. In embodiments, the cytokine is IL-15. In embodiments, the cytokine is IL-18. In embodiments, the natural killer cell-activating agent is a feeder cell.

In embodiments, deriving includes genetically engineering PD-L1 expression in the population of isolated natural killer cells. In embodiments, the population of PD-L1(+) natural killer cells is expanded prior to administering to the patient.

In embodiments, the method further includes detecting an amount of PD-L1(+) natural killer cells in a biological sample obtained from the patient prior to administering the PD-L1(+) natural killer cells. In embodiments, the biological sample includes PD-L1 (+) natural killer cells, has no PD-L1 (+) natural killer cells, has a natural killer cell deficiency, or has natural killer cell suppression. In embodiments, the biological sample includes PD-L1 (+) natural killer cells. In embodiments, the biological sample does not include PD-L1 (+) natural killer cells. In embodiments, the biological sample has a natural killer cell deficiency. In embodiments, the biological sample has natural killer cell suppression. In embodiments, the biological sample includes an amount of PD-L1(+) natural killer cells that is about equal to or greater than the amount of PD-L1(−) natural killer cells. In embodiments, the biological sample includes an amount of PD-L1(+) natural killer cells that is about equal to the amount of PD-L1(−) natural killer cells. In embodiments, the biological sample includes an amount of PD-L1(+) natural killer cells that is greater than the amount of PD-L1(−) natural killer cells.

In embodiments, detecting includes a method selected from the group consisting of flow cytometry, fluorescence-activated cell sorting, antibody cell staining, immunohistochemistry, reverse transcriptase-quantitative polymerase chain reaction, immunofluorescent assay, and a combination of two or more thereof. In embodiments, detecting includes flow cytometry. In embodiments, detecting includes fluorescence-activated cell sorting. In embodiments, detecting includes antibody cell staining. In embodiments, detecting includes immunohistochemistry. In embodiments, detecting includes reverse transcriptase-quantitative polymerase chain reaction. In embodiments, detecting includes immunofluorescent assay.

For the methods provided herein, in embodiments, the patient is a newly diagnosed cancer patient, a patient relapsed from a cancer treatment, or a patient that has undergone hematopoietic stem cell transplantation. In embodiments, the patient is a newly diagnosed cancer patient. In embodiments, the patient has relapsed from a cancer treatment. In embodiments, the patient has undergone hematopoietic stem cell transplantation.

In embodiments, the method further includes administering to the patient a natural killer cell activating agent. In embodiments, the natural killer cell-activating agent is a feeder cell. In embodiments, the feeder cell is a K562 cell, a K562 cell expressing IL-15, a K562 cell expressing IL-21, or a K562 cell expressing IL-15 and IL-21. In embodiments, the feeder cell is a K562 cell. In embodiments, the feeder cell is a K562 cell expressing IL-15. In embodiments, the feeder cell is a K562 cell expressing IL-21. In embodiments, the feeder cell is a K562 cell expressing IL-15 and IL-21. In embodiments, the natural killer cell-activating agent is a cytokine. In embodiments, the cytokine is IL-2, IL-12, IL-15, IL-18, or a combination of two or more thereof. In embodiments, the cytokine is IL-2. In embodiments, the cytokine is IL-12. In embodiments, the cytokine is IL-15. In embodiments, the cytokine is IL-18.

In embodiments, the methods provided herein include detecting an amount of PD-L1(+) natural killer (NK) cells in a biological sample from a subject. In embodiments, methods of detecting include flow cytometry, fluorescence-activated cell sorting, antibody cell staining, immunohistochemistry (IHC), reverse transcriptase-quantitative polymerase chain reaction (RT-qPCR), immunofluorescent assay, and a combination thereof. In embodiments, the method of detecting is flow cytometry. In embodiments, the method of detecting is fluorescence-activated cell sorting. In embodiments, the method of detecting is antibody cell staining. In embodiments, the method of detecting is immunohistochemistry (IHC). In embodiments, the method of detecting is reverse transcriptase-quantitative polymerase chain reaction (RT-qPCR). In embodiments, the method of detecting is immunofluorescent assay. In embodiments, the method of detecting is a combination of flow cytometry, fluorescence-activated cell sorting, antibody cell staining, immunohistochemistry (IHC), reverse transcriptase-quantitative polymerase chain reaction (RT-qPCR), and/or immunofluorescent assay.

In embodiments, the amount of PD-L1(+) NK cells is about equal to or greater than the amount of PD-L1(−) NK cells. In embodiments, the amount of PD-L1(+) NK cells is about equal to the amount of PD-L1(−) NK cells. In embodiments, the amount of PD-L1(+) NK cells is greater than the amount of PD-L1(−) NK cells. In embodiments, the amount of PD-L1+ NK cells in the biological sample from the subject is compared to the amount of PD-L1(−) NK cells in the same sample. In embodiments, the amount of PD-L1+ NK cells in the biological sample from the subject is compared to a control. In embodiments, the control is an amount of PD-L1(+) NK cells (e.g. average amount) found in healthy patients, cancer patients or the general population. In embodiments, the control is an amount of PD-L1(+) NK cells (e.g. average amount) found in healthy patients. In embodiments, the control is an amount of PD-L1(+) NK cells (e.g. average amount) found in cancer patients. In embodiments, the control is an amount of PD-L1(+) NK cells (e.g. average amount) found in general population.

In embodiments, the amount of PD-L1(+) NK cells correlates with response to anti-cancer therapy in that higher amounts of PD-L1(+) NK cells in a subject correlates to a higher probability that the subject will respond to anti-cancer therapy (e.g., experience a decrease in the number of cancer cells or tumor size). In embodiments, more PD-L1(+) NK cells than PD-L1(−) NK cells correlates with increased response to anti-cancer therapy. In embodiments, more PD-L1(+) NK cells than PD-L1(−) NK cells correlates with a better response to anti-cancer therapy.

In embodiments, the methods provided herein include administration of an anticancer therapy. In embodiments, the anticancer therapy is selected from chemotherapy, radiation therapy, surgery, targeted therapy, immunotherapy, and cell therapy. In embodiments, the anticancer therapy is chemotherapy. In embodiments, the anticancer therapy is radiation therapy. In embodiments, the anticancer therapy is surgery. In embodiments, the anticancer therapy is targeted therapy. In embodiments, the anticancer therapy is immunotherapy. In embodiments, the anticancer therapy is cell therapy. In embodiments, the method includes administration of an effective amount of an anti-cancer therapy in combination with an effective amount of PD-L1(+) NK cells provided herein including embodiments thereof. In embodiments, the method includes administration of an effective amount of an anti-cancer agent in combination with an effective amount of PD-L1(+) NK cells provided herein including embodiments thereof.

In embodiments, immunotherapy includes a checkpoint inhibitor (e.g. administration of an effective amount of a checkpoint inhibitor to the subject). In embodiments, the checkpoint inhibitor is a PD-1 inhibitor (e.g. administration of an effective amount of a PD-1 inhibitor to the subject). In embodiments, the PD-1 inhibitor is selected from pembrolizumab and nivolumab (e.g. administration of an effective amount of pembrolizumab or nivolumab to the subject). In embodiments, the PD-1 inhibitor is pembrolizumab (e.g. administration of an effective amount of pembrolizumab to the subject). In embodiments, the PD-1 inhibitor is nivolumab (e.g. administration of an effective amount of nivolumab to the subject). In embodiments, the checkpoint inhibitor is a PD-L1 inhibitor (e.g. administration of an effective amount of a PD-L1 inhibitor to the subject). In embodiments, the PD-L1 inhibitor is selected from atezolizumab, avelumab, and durvalumab (e.g. administration of an effective amount of atezolizumab, avelumab or durvalumab to the subject). In embodiments, the PD-L1 inhibitor is atezolizumab (e.g. administration of an effective amount of atezolizumab to the subject). In embodiments, the PD-L1 inhibitor is avelumab (e.g. administration of an effective amount of avelumab to the subject). In embodiments, the PD-L1 inhibitor is durvalumab (e.g. administration of an effective amount of to the subject).

In embodiments, the cell therapy includes PD-L1(+) NK cells. In embodiments, cell therapy includes administering cells such as NK cells directly into a subject. In embodiments, the NK cells express PD-L1 (denoted PD-L1(+)). In embodiments, the PD-L1(+) NK cells are enriched or purified. In embodiments, the PD-L1(+) NK cells are enriched. In embodiments, the PD-L1(+) NK cells are purified. In embodiments, enrichment and/or purification is achieved by obtaining NK cells from a mixture. Methods for enrichment and/or purification include but are not limited to cell separation based on cell density, size, and/or affinity for antibody-coated beads. The methods include, for example, adherence, filtration, centrifugation, panning, MACS (magnetic-activated cell sorting), and FACS (fluorescence activated cell sorting). In embodiments, the cell therapy includes bulk NK cells. In embodiments, the bulk NK cells include PD-L1(+) NK cells.

In embodiments, the anticancer therapy includes a checkpoint inhibitor and cell therapy. In embodiments, the anticancer therapy includes PD-L1 inhibitor and PD-L1(+) NK cells. In embodiments, the PD-L1 inhibitor is selected from atezolizumab, avelumab, and durvalumab and the PD-L1(+) NK cells are enriched or purified. In embodiments, the PD-L1 inhibitor is atezolizumab and the PD-L1(+) NK cells are enriched. In embodiments, the PD-L1 inhibitor is atezolizumab and the PD-L1(+) NK cells are purified. In embodiments, the anticancer therapy includes a checkpoint inhibitor and a cell therapy including bulk NK cells that include PD-L1(+) NK cells. In embodiments, the anticancer therapy includes a PD-L1 inhibitor and a cell therapy including bulk NK cells that include PD-L1(+) NK cells. In embodiments, the anticancer therapy includes atezolizumab and bulk NK cells that include PD-L1(+) NK cells.

In embodiments, the anticancer therapy includes a checkpoint inhibitor and an NK cell activating agent (e.g. administration of an effective amount of a checkpoint inhibitor and an NK cell activating agent to the subject). In embodiments, the checkpoint inhibitor is a PD-1 inhibitor. In embodiments, the PD-1 inhibitor is selected from pembrolizumab and nivolumab. In embodiments, the PD-1 inhibitor is pembrolizumab. In embodiments, the PD-1 inhibitor is nivolumab. In embodiments, the checkpoint inhibitor is a PD-L1 inhibitor. In embodiments, the PD-L1 inhibitor is selected from atezolizumab, avelumab, and durvalumab. In embodiments, the PD-L1 inhibitor is atezolizumab. In embodiments, the NK cell-activating agent is a cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18. In embodiments, the anticancer therapy includes pembrolizumab and IL-2. In embodiments, the anticancer therapy includes pembrolizumab and IL-12. In embodiments, the anticancer therapy includes pembrolizumab and IL-15. In embodiments, the anticancer therapy includes pembrolizumab and IL-18. In embodiments, the anticancer therapy includes pembrolizumab and a combination of IL-2, IL-12, IL-15, and/or IL-18. In embodiments, the anticancer therapy includes nivolumab and IL-2. In embodiments, the anticancer therapy includes nivolumab and IL-12. In embodiments, the anticancer therapy includes nivolumab and IL-15. In embodiments, the anticancer therapy includes nivolumab and IL-18. In embodiments, the anticancer therapy includes nivolumab and a combination of IL-2, IL-12, IL-15, and/or IL-18. In embodiments, the anticancer therapy includes avelumab and IL-2. In embodiments, the anticancer therapy includes avelumab and IL-12. In embodiments, the anticancer therapy includes avelumab and IL-15. In embodiments, the anticancer therapy includes avelumab and IL-18. In embodiments, the anticancer therapy includes avelumab and a combination of IL-2, IL-12, IL-15, and/or IL-18. In embodiments, the anticancer therapy includes durvalumab and IL-2. In embodiments, the anticancer therapy includes durvalumab and IL-12. In embodiments, the anticancer therapy includes avelumab and IL-15. In embodiments, the anticancer therapy includes durvalumab and IL-18. In embodiments, the anticancer therapy includes durvalumab and a combination of IL-2, IL-12, IL-15, and/or IL-18. n embodiments, the anticancer therapy includes atezolizumab and IL-2. In embodiments, the anticancer therapy includes atezolizumab and IL-12. In embodiments, the anticancer therapy includes atezolizumab and IL-15. In embodiments, the anticancer therapy includes atezolizumab and IL-18. In embodiments, the anticancer therapy includes atezolizumab and a combination of IL-2, IL-12, IL-15, and/or IL-18.

In embodiments, the methods of treating cancer described herein further comprise isolating natural killer (NK) cells from a subject thereby producing a population of isolated NK cells, deriving a population of PD-L1(+) NK cell from the population of isolated NK cells, and administering the population of PD-L1(+) NK cells (e.g. an effective amount of PD-L1(+) NK cells) into the patient. In embodiments, methods of isolating natural killer cells include obtaining NK cells from a biological sample from a subject. Methods for isolating natural killer cells include but are not limited to cell separation based on cell density, size, and/or affinity for antibody-coated beads. The methods include, for example, adherence, filtration, centrifugation, panning, MACS (magnetic-activated cell sorting), and FACS (fluorescence activated cell sorting). In embodiments, deriving a population of PD-L1(+) NK cells from the isolated natural killer cells include isolating, enriching, and/or purifying PD-L1(+) cells. Such methods include but are not limited to cell separation based on cell density, size, and/or affinity for antibody-coated beads. The methods include, for example, adherence, filtration, centrifugation, panning, MACS (magnetic-activated cell sorting), and FACS (fluorescence activated cell sorting). In embodiments, the PD-L1(+) NK cells are administered to the patient. In embodiments, deriving a population of PD-L1(+) cells from a population of NK cells includes genetically engineering expression of PD-L1 in the NK cells. Such methods of genetic engineering are known and include recombinant protein expression in human cells. Specifically, NK cells may be transfected with an expression vector capable of expressing functional PD-L1, thereby producing PD-L1(+) NK cells. In embodiments, the cancer is a cancer or tumor as described above.

In embodiments, the patient is selected from a patient diagnosed with cancer, a cancer patient relapsed from a treatment, or a cancer patient that has undergone hematopoietic stem cell transplantation. In embodiments, the patient is a patient diagnosed with cancer. In embodiments, the patient is a cancer patient relapsed from a treatment. In embodiments, the patient is a cancer patient that has undergone hematopoietic stem cell transplantation. In embodiments, the patient has PD-L1 (+) NK cells, has no PD-L1 (+) NK cells, has an NK cell deficiency, or has NK cell suppression. In embodiments, the patient has PD-L1 (+) NK cells. In embodiments, the patient has no PD-L1 (+) NK cells. In embodiments, having no PD-L1 (+) NK cells includes having no detectable levels of PD-L1(+) NK cells. In embodiments, have no PD-L1 (+) NK cells includes having low levels of PD-L1(+) cells compared to a control. In embodiments, the control is a reference number of PD-L1(+) cells. In embodiments, the control is the average or mean number of PD-L1(+) cells in a healthy individual. In embodiments, the patient has an NK cell deficiency. In embodiments, the patient has NK cell suppression. In embodiments, NK cell suppression includes reduced NK cell activity, reduced NK cell number, and or reduced NK cell function.

In embodiments, the methods of treating cancer described herein further comprise isolating natural killer (NK) cells from a subject thereby producing a population of isolated NK cells. In embodiments, methods of isolating NK cells include obtaining a population of cells from a subject where the population of cells includes NK cells. In embodiments, NK cells are isolated from the population of cells by any known method including but not limited to fluorescence-activated cell sorting, magnetic bead separation, and/or column purification. In embodiments, the method of isolating NK cells is fluorescence-activated cell sorting. In embodiments, the method of isolating NK cells is magnetic bead separation. In embodiments, the method of isolating NK cells is column purification. In embodiments, the method of isolating NK cells is a combination of fluorescence-activated cell sorting, magnetic bead separation, and/or column purification.

In embodiments, the methods of treating cancer described herein further comprise include isolating NK cells from a subject. In embodiments, the subject is selected from an autologous cancer patient, a healthy donor, a matched heterologous hematopoietic stem cell donor, and a partially matched heterologous hematopoietic stem cell donor. In embodiments, the subject is an autologous cancer patient. The term “autologous cancer patient” refers to a cancer subject who is to be treated with methods of treating cancer described herein. In embodiments, the subject is a healthy donor. In embodiments, the healthy donor is a blood donor. In embodiments, the healthy donor is a PBMC (peripheral blood mononuclear cell) donor. In embodiments, the subject is a matched heterologous hematopoietic stem cell donor. The term “matched heterologous hematopoietic stem cell donor” refers to a subject from which NK cells are isolated has matching tissue type as the patient to be treated. Matching tissue type can be HLA type. In embodiments, the subject is a partially matched heterologous hematopoietic stem cell donor. The term “partially matched heterologous hematopoietic stem cell donor” refers to a subject from which NK cells are isolated has partially matching tissue type as the patient to be treated. Matching tissue type can be HLA type.

In embodiments, the methods of treating cancer described herein further comprise deriving a population of PD-L1(+) NK cells from the population of isolated NK cells. In embodiments, the method of deriving includes expanding PD-L1(+) NK cells by exposing the isolated NK cells to a feeder cell thereby producing a population of PD-L1(+) NK cell. In embodiments, the feeder cell is a K562 cell. In embodiments, the feeder cell is a K562 cell expressing IL-15 and IL-21.

In embodiments, the method of deriving a population of PD-L1(+) NK cells includes fluorescence-activated cell sorting, magnetic bead separation, and/or column purification thereby producing a population of PD-L1(+) NK cell. The methods include obtaining PD-L1(+) cells from a mixture of cells in a sample. The methods may be based on separation by cell density, size, and/or affinity for antibody-coated beads. The methods include, for example, adherence, filtration, centrifugation, panning, MACS (magnetic-activated cell sorting), and FACS (Fluorescence activated cell sorting. In embodiments, the method of deriving is fluorescence-activated cell sorting. In embodiments, the method of deriving is magnetic bead separation. In embodiments, the method of deriving is column purification.

In embodiments, the methods of deriving a population of PD-L1(+) NK cells include exposing the isolated NK cells to an NK activating agent to induce PD-L1 expression thereby producing a population of PD-L1(+) NK cell. In embodiments, the NK cell-activating agent is a feeder cell. In embodiments, exposing includes co-culturing isolated NK cells with a feeder cell. In embodiments, the feeder cell is a K562 cell. In embodiments, the feeder cell is a K562 cell expressing IL-15 and IL-21. In embodiments, exposing includes adding NK cell-activating an agent. In embodiments, the NK cell-activating agent is a cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18.

In embodiments, the methods of treating cancer described herein further comprise expanding the population of PD-L1(+) NK cells prior to administering into the patient. Methods of expanding PD-L1(+) NK cells include exposing the PD-L1(+)NK cells to NK activating agents as described herein.

In embodiments, the methods of treating cancer described herein further comprise deriving a population of PD-L1(+)NK cells include genetically engineering PD-L1 expression in the population of isolated NK cells thereby producing a population of PD-L1(+) NK cell. Such methods of genetic engineering are known and include recombinant protein expression in human cells. Specifically, NK cells may be transfected with an expression vector capable of expressing functional PD-L1, thereby producing PD-L1(+) NK cells.

In embodiments, the methods of treating cancer described herein further comprise administering an anticancer therapy (e.g. administration of an effective amount of an anticancer compound or chemotherapeutic agent to the subject). The anticancer therapy may include chemotherapy, radiation therapy, surgery, targeted therapy, immunotherapy, cell therapy, and/or a combination thereof. In embodiments, the methods provided herein further include administering chemotherapy (e.g. administration of an effective amount of the therapy to the subject). In embodiments, the methods provided herein further include administering radiation therapy. In embodiments, the methods provided herein further include administering surgery. In embodiments, the methods provided herein further include administering targeted therapy. In embodiments, the methods provided herein further include administering immunotherapy (e.g. administration of an effective amount of an immunotherapeutic agent to the subject). In embodiments, the methods provided herein further include administering cell therapy (e.g. administration of an effective amount of therapeutic cells to the subject). In embodiments, the methods provided herein further include administering a combination of chemotherapy, radiation therapy, surgery, targeted therapy, immunotherapy and cell therapy.

In embodiments, the immunotherapy includes administering an effective amount of a checkpoint inhibitor (e.g. administration of an effective amount of a checkpoint inhibitor to the subject). In embodiments, the checkpoint inhibitor is a PD-1 inhibitor (e.g. administration of an effective amount of a PD-1 inhibitor to the subject). In embodiments, the PD-1 inhibitor is selected from pembrolizumab and nivolumab (e.g. administration of an effective amount of pembrolizumab or nivolumab to the subject). In embodiments, the PD-1 inhibitor is pembrolizumab (e.g. administration of an effective amount of pembrolizumab to the subject). In embodiments, the PD-1 inhibitor is nivolumab (e.g. administration of an effective amount of nivolumab to the subject). In embodiments, the checkpoint inhibitor is a PD-L1 inhibitor (e.g. administration of an effective amount of a PD-L1 inhibitor to the subject). In embodiments, the PD-L1 inhibitor is selected from atezolizumab, avelumab, and durvalumab (e.g. administration of an effective amount of atezolizumab, avelumab, or durvalumab to the subject). In embodiments, the PD-L1 inhibitor is atezolizumab (e.g. administration of an effective amount of atezolizumab to the subject). In embodiments, the PD-L1 inhibitor is avelumab (e.g. administration of an effective amount of avelumab to the subject). In embodiments, the PD-L1 inhibitor is durvalumab (e.g. administration of an effective amount of to the subject).

In embodiments, the anticancer therapy includes administering an effective amount of an NK cell-activating agent. In embodiments, the NK cell-activating agent cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18.

In embodiments, the methods of treating cancer described herein further comprise administering an NK cell-activating agent and an immunotherapeutic agent in a combined effective amount to the subject. In embodiments, the NK cell-activating agent is a cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18.

In embodiments, immunotherapy includes a checkpoint inhibitor (e.g. administration of an effective amount of a checkpoint inhibitor to the subject). In embodiments, the checkpoint inhibitor is a PD-1 inhibitor (e.g. administration of an effective amount of a PD-1 inhibitor to the subject). In embodiments, the PD-1 inhibitor is selected from pembrolizumab and nivolumab (e.g. administration of an effective amount of pembrolizumab or nivolumab to the subject). In embodiments, the PD-1 inhibitor is pembrolizumab (e.g. administration of an effective amount of pembrolizumab to the subject). In embodiments, the PD-1 inhibitor is nivolumab (e.g. administration of an effective amount of nivolumab to the subject). In embodiments, the checkpoint inhibitor is a PD-L1 inhibitor (e.g. administration of an effective amount of a PD-L1 inhibitor to the subject). In embodiments, the PD-L1 inhibitor is selected from atezolizumab, avelumab, and durvalumab (e.g. administration of an effective amount of atezolizumab, avelumab or durvalumab to the subject). In embodiments, the PD-L1 inhibitor is atezolizumab (e.g. administration of an effective amount of atezolizumab to the subject). In embodiments, the PD-L1 inhibitor is avelumab (e.g. administration of an effective amount of avelumab to the subject). In embodiments, the PD-L1 inhibitor is durvalumab (e.g. administration of an effective amount of to the subject).

In embodiments, the methods of treating cancer described herein further comprise administering an NK cell-activating cytokine and an immunotherapeutic agent in combined effective amount. In embodiments, methods of treating cancer in a subject include administering an NK cell-activating cytokine and a checkpoint inhibitor in a combined effective amount. In embodiments, methods of treating cancer in a subject include administering an NK cell-activating cytokine selected from IL-2, IL-12, IL-15, IL-18, and a combination thereof and a checkpoint inhibitor selected from a PD-1 inhibitor and a PD-L1 inhibitor in a combined effective amount.

In embodiments, the methods of treating cancer described herein further comprise administering an NK cell-activating cytokine selected from IL-2, IL-12, IL-15, IL-18, and a combination thereof and a PD-1 inhibitor selected from pembrolizumab and nivolumab, in a combined effective amount. In embodiments, methods of treating cancer in a subject include administering IL-2, IL-12, IL-15, or IL-18 and pembrolizumab or nivolumab, in a combined effective amount. In embodiments, methods of treating cancer in a subject including administering IL-2 and pembrolizumab, in a combined effective amount. In embodiments, methods of treating cancer in a subject include administering IL-2 and nivolumab, in a combined effective amount. In embodiments, methods of treating cancer in a subject include administering IL-12 and pembrolizumab, in a combined effective amount. In embodiments, methods of treating cancer in a subject include administering IL-12 and nivolumab, in a combined effective amount. In embodiments, methods of treating cancer in a subject include administering IL-15 and pembrolizumab, in a combined effective amount. In embodiments, methods of treating cancer in a subject include administering IL-15 and nivolumab, in a combined effective amount. In embodiments, methods of treating cancer in a subject include administering IL-18 and pembrolizumab, in a combined effective amount. In embodiments, methods of treating cancer in a subject include administering IL-18 and nivolumab, in a combined effective amount. In embodiments, methods of treating cancer in a subject include administering a combination of IL-2, IL-12, IL-15, and/or IL-18 and pembrolizumab or nivolumab, in a combined effective amount.

In embodiments, the methods of treating cancer described herein further comprise administering IL-2, IL-12, IL-15, or IL-18 and atezolizumab, avelumab, or durvalumab, in a combined effective amount. In embodiments, methods of treating cancer in a subject include administering IL-2 and atezolizumab, in a combined effective amount. In embodiments, methods of treating cancer in a subject include administering IL-2 and avelumab, in a combined effective amount. In embodiments, methods of treating cancer in a subject include administering IL-2 and durvalumab, in a combined effective amount. In embodiments, methods of treating cancer in a subject include administering IL-12 and atezolizumab, in a combined effective amount. In embodiments, methods of treating cancer in a subject include administering IL-12 and avelumab, in a combined effective amount. In embodiments, methods of treating cancer in a subject include administering IL-12 and durvalumab. In embodiments, methods of treating cancer in a subject include administering IL-15 and atezolizumab, in a combined effective amount. In embodiments, methods of treating cancer in a subject including administering IL-15 and avelumab, in a combined effective amount. In embodiments, methods of treating cancer in a subject include administering IL-15 and durvalumab, in a combined effective amount. In embodiments, methods of treating cancer in a subject include administering IL-18 and atezolizumab, in a combined effective amount. In embodiments, methods of treating cancer in a subject include administering IL-18 and avelumab, in a combined effective amount. In embodiments, methods of treating cancer in a subject include administering IL-18 and durvalumab, in a combined effective amount. In embodiments, methods of treating cancer in a subject include administering a combination of IL-2, IL-12, IL-15, and/or IL-18 and atezolizumab, avelumab, or durvalumab, in a combined effective amount.

### Nucleic Acid Compositions

In an aspect is provided a nucleic acid encoding an soluble IL-15 protein, wherein the soluble IL-15 protein includes an IL-2 signal peptide and an IL-15 protein. In embodiments, the nucleic acid includes the sequence of SEQ ID NO:1. In embodiments, the nucleic acid includes the sequence of SEQ ID NO:2. In embodiments, the nucleic acid includes the sequence of SEQ ID NO:3. In embodiments, the nucleic acid is the sequence of SEQ ID NO:3.

### Recombinant Soluble IL-15 Proteins

In an aspect is provided a recombinant soluble IL-15 protein, wherein the soluble IL-15 protein includes an IL-2 signal peptide and an IL-15 protein. In embodiments, the soluble IL-15 protein includes the sequence of SEQ ID NO:4. In embodiments, the soluble IL-15 protein includes the sequence of SEQ ID NO:5. In embodiments, the soluble IL-15 protein includes the sequence of SEQ ID NO:6. In embodiments, the soluble IL-15 protein includes the sequence of SEQ ID NO:6.

### P EMBODIMENTS

P Embodiment 1. A method of treating non-small cell lung cancer in a patient in need thereof, the method comprising administering to the patient an effective amount of PD-L1(+) natural killer cells once per week; wherein the PD-L1(+) natural killer cells express soluble IL-15.

P Embodiment 2. A method of treating non-small cell lung cancer in a patient in need thereof, the method comprising administering to the patient from about 1×10⁶ to about 1×10¹² of PD-L1(+) natural killer cells; wherein the PD-L1(+) natural killer cells express soluble IL-15.

P Embodiment 3. A method of treating non-small cell lung cancer in a patient in need thereof, the method comprising administering to the patient from about 1×10⁶ to about 1×10¹² of PD-L1(+) natural killer cells once per week; wherein the PD-L1(+) natural killer cells express soluble IL-15.

P Embodiment 4. A method of treating non-small cell lung cancer in a patient in need thereof, the method comprising administering to the patient: (i) from about 1×10⁶ to about 1×10¹² of PD-L1(+) natural killer cells once per week; wherein the PD-L1(+) natural killer cells express soluble IL-15; and (ii) an effective amount of atezolizumab.

P Embodiment 5. The method of any one of P Embodiments 1 to 4, comprising administering from about 1×10⁷ to about 1×10¹² of the PD-L1(+) natural killer cells.

P Embodiment 6. The method of any one of P Embodiments 1 to 4, comprising administering from about 2×10⁷ to about 1×10¹⁰ of the PD-L1(+) natural killer cells.

P Embodiment 7. The method of any one of P Embodiments 1 to 4, comprising administering from about 4×10⁷ to about 2×10⁹ of the PD-L1(+) natural killer cells.

P Embodiment 8. The method of any one of P Embodiments 1 to 4, comprising administering from about 1×10⁸ to about 2×10⁹ of the PD-L1(+) natural killer cells.

P Embodiment 9. The method of any one of P Embodiments 1 to 4, comprising administering about 5×10⁷ of the PD-L1(+) natural killer cells.

P Embodiment 10. The method of any one of P Embodiments 1 to 4, comprising administering about 1×10⁸ of the PD-L1(+) natural killer cells.

P Embodiment 11. The method of any one of P Embodiments 1 to 4, comprising administering about 2×10⁸ of the PD-L1(+) natural killer cells.

P Embodiment 12. The method of any one of P Embodiments 1 to 4, comprising administering about 4×10⁸ of the PD-L1(+) natural killer cells.

P Embodiment 13. The method of any one of P Embodiments 1 to 4, comprising administering about 5×10⁸ of the PD-L1(+) natural killer cells.

P Embodiment 14. The method of any one of P Embodiments 1 to 4, comprising administering about 1×10⁹ of the PD-L1(+) natural killer cells.

P Embodiment 15. The method of any one of P Embodiments 1 to 14, wherein the effective amount of PD-L1(+) natural killer cells are administered to the patient once per week for four weeks.

P Embodiment 16. The method of P Embodiment 15, wherein administration of the PD-L1(+) natural killer cells is discontinued for 4 weeks; wherein another 8 week treatment cycle is optionally initiated one or more times thereafter.

P Embodiment 17. The method of any one of P Embodiments 1 to 14, wherein the effective amount of PD-L1(+) natural killer cells are administered to the patient once per week for five weeks.

P Embodiment 18. The method of P Embodiment 17, wherein administration of the PD-L1(+) natural killer cells is discontinued for 3 weeks; wherein another 8 week treatment cycle is optionally initiated one or more times thereafter.

P Embodiment 19. The method of any one of P Embodiments 1 to 14, wherein the effective amount of PD-L1(+) natural killer cells are administered to the patient once per week for six weeks.

P Embodiment 20. The method of P Embodiment 19, wherein administration of the PD-L1(+) natural killer cells is discontinued for 2 weeks; wherein another 8 week treatment cycle is optionally initiated one or more times thereafter.

P Embodiment 21. The method of any one of P Embodiments 1 to 14, wherein the effective amount of PD-L1(+) natural killer cells are administered to the patient once per week for seven weeks.

P Embodiment 22. The method of P Embodiment 21, wherein administration of the PD-L1(+) natural killer cells is discontinued for 1 week; wherein another 8 week treatment cycle is optionally initiated one or more times thereafter.

P Embodiment 23. The method of any one of P Embodiments 1 to 14, wherein the effective amount of PD-L1(+) natural killer cells are administered to the patient once per week for eight weeks.

P Embodiment 24. The method of any one of P Embodiments 1 to 14, wherein the effective amount of PD-L1(+) natural killer cells are administered to the patient twice per week for four weeks.

P Embodiment 25. The method of P Embodiment 24, wherein administration of the PD-L1(+) natural killer cells is discontinued for 4 weeks; wherein another 8 week treatment cycle is optionally initiated one or more times thereafter.

P Embodiment 26. The method of any one of P Embodiments 1 to 14, wherein the effective amount of PD-L1(+) natural killer cells are administered to the patient twice per week for five weeks.

P Embodiment 27. The method of P Embodiment 26, wherein administration of the PD-L1(+) natural killer cells is discontinued for 3 weeks; wherein another 8 week treatment cycle is optionally initiated one or more times thereafter.

P Embodiment 28. The method of any one of P Embodiments 1 to 14, wherein the effective amount of PD-L1(+) natural killer cells are administered to the patient twice per week for six weeks.

P Embodiment 29. The method of P Embodiment 28, wherein administration of the PD-L1(+) natural killer cells is discontinued for 2 weeks; wherein another 8 week treatment cycle is optionally initiated one or more times thereafter.

P Embodiment 30. The method of any one of P Embodiments 1 to 14, wherein the effective amount of PD-L1(+) natural killer cells are administered to the patient twice per week for seven weeks.

P Embodiment 31. The method of P Embodiment 30, wherein administration of the PD-L1(+) natural killer cells is discontinued for 1 week; wherein another 8 week treatment cycle is optionally initiated one or more times thereafter.

P Embodiment 32. The method of any one of P Embodiments 1 to 14, wherein the effective amount of PD-L1(+) natural killer cells are administered to the patient twice per week for eight weeks.

P Embodiment 33. The method of any one of P Embodiments 1 to 32, wherein the PD-L1(+) natural killer cells are administered intravenously as an infusion for about 30 minutes to about 120 minutes.

P Embodiment 34. The method of P Embodiment 33, wherein the PD-L1(+) natural killer cells are administered intravenously as an infusion for about 60 minutes to about 90 minutes.

P Embodiment 35. The method of any one of P Embodiments 1 to 34, wherein PD-L1(+) natural killer cells are activated cord blood natural killer cells that have been genetically modified to constitutively express soluble IL-15.

P Embodiment 36. The method of any one of P Embodiments 1 to 35, wherein PD-L1(+) natural killer cells express cell surface PD-L1.

P Embodiment 37. The method of any one of P Embodiments 1 to 36, wherein PD-L1(+) natural killer cells express truncated EGFR.

P Embodiment 38. The method of any one of P Embodiments 1 to 37, wherein PD-L1(+) natural killer cells were derived from umbilical cord blood natural killer cells.

P Embodiment 39. The method of P Embodiment 38, wherein the umbilical cord blood natural killer cells were incubated with IL-12 and IL-18.

P Embodiment 40. The method of any one of P Embodiments 1 to 39, wherein PD-L1(+) natural killer cells do not express a CD19 chimeric antigen receptor.

P Embodiment 41. The method of any one of P Embodiments 1 to 40, further comprising pretreating the patient with a low-dose and/or escalating dose of the PD-L1(+) natural killer cells prior to beginning treatment with the effective dose, wherein the low-dose and/or escalating dose is an amount lower than the effective dose.

P Embodiment 42. The method of any one of P Embodiments 1-41, further comprising administering to the patient an effective amount of a checkpoint inhibitor.

P Embodiment 43. The methods of P Embodiment 42, wherein the checkpoint inhibitor is a PD-1 inhibitor.

P Embodiment 44. The method of P Embodiment 43, wherein the PD-1 inhibitor is pembrolizumab, nivolumab, or a combination thereof.

P Embodiment 45. The methods of P Embodiment 42, wherein the checkpoint inhibitor is a PD-L1 inhibitor.

P Embodiment 46. The method of P Embodiment 45, wherein the PD-L1 inhibitor is atezolizumab, avelumab, or durvalumab.

P Embodiment 47. The method of P Embodiment 46, wherein the PD-L1 inhibitor is atezolizumab.

P Embodiment 48. The method of P Embodiment 4 or 47, comprising administering to the patient about 840 mg of atezolizumab about once every 14 days.

P Embodiment 49. The method of P Embodiment 4 or 47, comprising administering to the patient about 120 mg of atezolizumab about once every 21 days.

P Embodiment 50. The method of P Embodiment 4 to 47, comprising administering to the patient about 1680 mg of atezolizumab about once every 28 days.

P Embodiment 51. The method of any one of P Embodiments 4 and 47-50, wherein the atezolizumab is administered to the patient by intravenous infusion.

P Embodiment 52. The method of P Embodiment 51, wherein atezolizumab is administered to the patient by intravenous infusion over a period of time from about 30 minutes to about 90 minutes.

P Embodiment 53. The method of P Embodiment 52, wherein atezolizumab is administered to the patient by intravenous infusion over a period of time of about 60 minutes.

P Embodiment 54. The method of any one of P Embodiments 1 to 53, wherein the non-small cell lung cancer is advanced non-small cell lung cancer.

P Embodiment 55. The method of any one of P Embodiments 1 to 54, wherein the non-small cell lung cancer is metastatic non-small cell lung cancer.

P Embodiment 56. The method of any one of P Embodiments 1 to 55, wherein the non-small cell lung cancer is recurrent non-small cell lung cancer.

P Embodiment 57. The method of any one of P Embodiments 1 to 56, wherein the non-small cell lung cancer comprises PD-L1(−) tumor cells.

P Embodiment 58. The method of any one of P Embodiments 1 to 57, wherein the non-small cell lung cancer comprises PD-L1(+) tumor cells.

P Embodiment 59. The method of any one of P Embodiments 1 to 58, wherein the patient has been treated with a PD-1 inhibitor and/or PD-L1 inhibitor prior to treatment with the PD-L1(+) natural killer cells.

P Embodiment 60. The method of any one of P Embodiments 1 to 59, wherein the patient is refractory to a PD-1 inhibitor and/or a PD-L1 inhibitor.

P Embodiment 61. The method of any one of P Embodiments 1 to 60, wherein the PD-L1(+) natural killer cells are produced by a process comprising the steps of: (a) isolating natural killer cells from a donor subject thereby producing a population of isolated natural killer cells; (b) deriving a population of PD-L1(+) natural killer cell from the population of isolated natural killer cells; thereby producing the PD-L1(+) natural killer cells (population of PD-L1(+) natural killer cells).

P Embodiment 62. The method of P Embodiment 61, further comprising enriching and purifying the population of PD-L1(+) natural killer cells after step (b).

P Embodiment 63. The method of P Embodiment 61 or 62, wherein isolating comprises fluorescence-activated cell sorting, magnetic bead separation, column purification, or a combination of two or more thereof.

P Embodiment 64. The method of any one of P Embodiments 61 to 63, wherein the donor subject is an autologous cancer patient, a healthy donor, a matched heterologous hematopoietic stem cell donor, or a partially matched heterologous hematopoietic stem cell donor.

P Embodiment 65. The method of any one of P Embodiments 61 to 64, wherein deriving comprises expanding the population of PD-L1(+) natural killer cells by exposing the population of isolated natural killer cells to a feeder cell.

P Embodiment 66. The method of P Embodiments 65, wherein the feeder cell is a K562 cell; a K562 cell expressing IL-15; a K562 cell expressing IL-21; or a K562 cell expressing IL-15 and IL-21.

P Embodiment 67. The method of any one of P Embodiments 61 to 66, wherein deriving comprises fluorescence-activated cell sorting, magnetic bead separation, column purification, or a combination of two or more thereof.

P Embodiment 68. The method of any one of P Embodiments 61 to 67, wherein deriving comprises exposing the population of isolated natural killer cells to an natural killer activating agent to induce PD-L1 expression.

P Embodiment 69. The method of P Embodiment 68, wherein the natural killer cell-activating agent is a cytokine.

P Embodiment 70. The method of P Embodiment 69, wherein the cytokine is IL-2, IL-12, IL-15, IL-18, or a combination of two or more thereof.

P Embodiment 71. The method of P Embodiment 68, wherein the natural killer cell-activating agent is a feeder cell.

P Embodiment 72. The method of any one of P Embodiments 51 to 71, wherein deriving comprises genetically engineering PD-L1 expression in the population of isolated natural killer cells.

P Embodiment 73. The method of any one of P Embodiments 61 to 72, wherein the population of PD-L1(+) natural killer cell is expanded prior to administering to the patient.

P Embodiment 74. The method of any one of P Embodiments 1 to 73, further comprising detecting an amount of PD-L1(+) natural killer cells in a biological sample obtained from the patient prior to administering the PD-L1(+) natural killer cells.

P Embodiment 75. The method of P Embodiment 74, wherein the biological sample comprises PD-L1 (+) natural killer cells, has no PD-L1 (+) natural killer cells, has a natural killer cell deficiency, or has natural killer cell suppression.

P Embodiment 76. The method of P Embodiment 74 or 75, wherein the biological sample comprises an amount of PD-L1(+) natural killer cells that is about equal to or greater than the amount of PD-L1(−) natural killer cells.

P Embodiment 77. The method of any one of P Embodiments 74 to 76, wherein detecting comprises a method selected from the group consisting of flow cytometry, fluorescence-activated cell sorting, antibody cell staining, immunohistochemistry, reverse transcriptase-quantitative polymerase chain reaction, immunofluorescent assay, and a combination of two or more thereof.

P Embodiment 78. The method of any one of P Embodiments 1 to 77, wherein the patient is a newly diagnosed cancer patient, a patient relapsed from a cancer treatment, or a patient that has undergone hematopoietic stem cell transplantation.

P Embodiment 79. The method of any one of P Embodiments 1 to 78, further comprising administering to the patient a natural killer cell activating agent.

P Embodiment 80. The method of P Embodiment 79, wherein the natural killer cell-activating agent is a feeder cell.

P Embodiment 81. The method of P Embodiment 80, wherein the feeder cell is a K562 cell, a K562 cell expressing IL-15, a K562 cell expressing IL-21, or a K562 cell expressing IL-15 and IL-21.

P Embodiment 82. The method of P Embodiment 79, wherein the natural killer cell-activating agent is a cytokine.

P Embodiment 83. The method of P Embodiment 82, wherein the cytokine is IL-2, IL-12, IL-15, IL-18, or a combination of two or more thereof.

### EMBODIMENTS

Embodiment 1. A PD-L1(+) natural killer cell, wherein the PD-L1(+) natural killer cell expresses soluble IL-15.

Embodiment 2. The PD-L1(+) natural killer cell of Embodiment 1, wherein the PD-L1(+) natural killer cell expresses soluble IL-15 and truncated EGFR.

Embodiment 3. The PD-L1(+) natural killer cell of Embodiment 1 or 2, wherein the PD-L1(+) natural killer cell is an activated cord blood natural killer cell.

Embodiment 4. The PD-L1(+) natural killer cell of any one of Embodiments 1 to 3, wherein the PD-L1(+) natural killer cell was derived from an umbilical cord blood natural killer cell.

Embodiment 5. The PD-L1(+) natural killer cell of any one of Embodiments 1 to 4, wherein the PD-L1(+) natural killer cell does not express a CD19 chimeric antigen receptor.

Embodiment 6. A population of PD-L1(+) natural killer cells, wherein the PD-L1(+) natural killer cells express soluble IL-15.

Embodiment 7. The population of PD-L1(+) natural killer cells of Embodiment 6, wherein the PD-L1(+) natural killer cells express soluble IL-15 and truncated EGFR.

Embodiment 8. The population of PD-L1(+) natural killer cells of Embodiment 6 or 7, wherein the PD-L1(+) natural killer cells are activated cord blood natural killer cells.

Embodiment 9. The population of PD-L1(+) natural killer cells of any one of Embodiments 6 to 8, wherein the PD-L1(+) natural killer cells were derived from umbilical cord blood natural killer cells.

Embodiment 10. The population of PD-L1(+) natural killer cells of any one of Embodiments 6 to 9, wherein the PD-L1(+) natural killer cells do not express a CD19 chimeric antigen receptor.

Embodiment 11. A pharmaceutical composition comprising the PD-L1(+) natural killer cell of any one of Embodiments 1 to 5.

Embodiment 12. A pharmaceutical composition comprising the population of PD-L1(+) natural killer cells of any one of Embodiments 6 to 10.

Embodiment 13. A method of treating cancer in a patient in need thereof, the method comprising administering to the patient an effective amount of the PD-L1(+) natural killer cell of any one of claims 1 to 5, the population of PD-L1(+) natural killer cells of any one of claims 6 to 10, or the pharmaceutical composition of Embodiment 11 or 12.

Embodiment 14. The method of Embodiment 13, wherein the effective amount is from about 1×10⁶ to about 1×10¹² of PD-L1(+) natural killer cells.

Embodiment 15. The method of Embodiment 13, wherein the effective amount is from about 1×10⁷ to about 1×10¹² of the PD-L1(+) natural killer cells.

Embodiment 16. The method of Embodiment 13, wherein the effective amount is from about 2×10⁷ to about 1×10¹⁰ of the PD-L1(+) natural killer cells.

Embodiment 17. The method of Embodiment 13, wherein the effective amount is from about 4×10⁷ to about 2×10⁹ of the PD-L1(+) natural killer cells.

Embodiment 18. The method of Embodiment 13, wherein the effective amount is from about 1×10⁸ to about 2×10⁹ of the PD-L1(+) natural killer cells.

Embodiment 19. The method of Embodiment 13, wherein the effective amount is about 5×10⁷ of the PD-L1(+) natural killer cells.

Embodiment 20. The method of Embodiment 13, wherein the effective amount is about 1×10⁸ of the PD-L1(+) natural killer cells.

Embodiment 21. The method of Embodiment 13, wherein the effective amount is about 2×10⁸ of the PD-L1(+) natural killer cells.

Embodiment 22. The method of Embodiment 13, wherein the effective amount is about 4×10⁸ of the PD-L1(+) natural killer cells.

Embodiment 23. The method of Embodiment 13, wherein the effective amount is about 5×10⁸ of the PD-L1(+) natural killer cells.

Embodiment 24. The method of Embodiment 13, wherein the effective amount is about 1×10⁹ of the PD-L1(+) natural killer cells.

Embodiment 25. The method of any one of Embodiments 13 to 24, comprising administering the PD-L1(+) natural killer cells to the patient about twice per week.

Embodiment 26. The method of any one of Embodiments 13 to 24, comprising administering the PD-L1(+) natural killer cells to the patient about once per day.

Embodiment 27. The method of any one of Embodiments 13 to 24, comprising administering the PD-L1(+) natural killer cells to the patient about once every two days.

Embodiment 28. The method of any one of Embodiments 13 to 24, comprising administering the PD-L1(+) natural killer cells to the patient about once every three days.

Embodiment 29. The method of any one of Embodiments 13 to 24, comprising administering the PD-L1(+) natural killer cells to the patient about once every four days.

Embodiment 30. The method of any one of Embodiments 13 to 24, comprising administering the PD-L1(+) natural killer cells to the patient about once every five days.

Embodiment 31. The method of any one of Embodiments 13 to 24, comprising administering the PD-L1(+) natural killer cells to the patient about once every six days.

Embodiment 32. The method of any one of Embodiments 13 to 24, comprising administering the PD-L1(+) natural killer cells to the patient about once every three or four days.

Embodiment 33. The method of any one of Embodiments 13 to 24, comprising administering the PD-L1(+) natural killer cells to the patient about once per week.

Embodiment 34. The method of any one of Embodiments 13 to 24, wherein the PD-L1(+) natural killer cells are administered to the patient once per week for four weeks.

Embodiment 35. The method of Embodiment 34, wherein administration of the PD-L1(+) natural killer cells is discontinued for 4 weeks; wherein another 8 week treatment cycle is optionally initiated one or more times thereafter.

Embodiment 36. The method of any one of Embodiments 13 to 24, wherein the effective amount of PD-L1(+) natural killer cells are administered to the patient once per week for five weeks.

Embodiment 37. The method of Embodiment 36, wherein administration of the PD-L1(+) natural killer cells is discontinued for 3 weeks; wherein another 8 week treatment cycle is optionally initiated one or more times thereafter.

Embodiment 38. The method of any one of Embodiments 13 to 24, wherein the PD-L1(+) natural killer cells are administered to the patient once per week for six weeks.

Embodiment 39. The method of Embodiment 38, wherein administration of the PD-L1(+) natural killer cells is discontinued for 2 weeks; wherein another 8 week treatment cycle is optionally initiated one or more times thereafter.

Embodiment 40. The method of any one of Embodiments 13 to 24, wherein the PD-L1(+) natural killer cells are administered to the patient once per week for seven weeks.

Embodiment 41. The method of Embodiment 40, wherein administration of the PD-L1(+) natural killer cells is discontinued for 1 week; wherein another 8 week treatment cycle is optionally initiated one or more times thereafter.

Embodiment 42. The method of any one of Embodiments 13 to 24, wherein the PD-L1(+) natural killer cells are administered to the patient once per week for eight weeks.

Embodiment 43. The method of Embodiment 42, wherein the PD-L1(+) natural killer cells are administered to the patient twice per week for four weeks.

Embodiment 44. The method of Embodiment 42 or 43, wherein administration of the PD-L1(+) natural killer cells is discontinued for 4 weeks; wherein another 8 week treatment cycle is optionally initiated one or more times thereafter.

Embodiment 45. The method of any one of Embodiments 13 to 24, wherein the PD-L1(+) natural killer cells are administered to the patient twice per week for five weeks.

Embodiment 46. The method of Embodiment 45, wherein administration of the PD-L1(+) natural killer cells is discontinued for 3 weeks; wherein another 8 week treatment cycle is optionally initiated one or more times thereafter.

Embodiment 47. The method of any one of Embodiments 13 to 24, wherein the PD-L1(+) natural killer cells are administered to the patient twice per week for six weeks.

Embodiment 48. The method of Embodiment 47, wherein administration of the PD-L1(+) natural killer cells is discontinued for 2 weeks; wherein another 8 week treatment cycle is optionally initiated one or more times thereafter.

Embodiment 49. The method of any one of Embodiments 13 to 24, wherein the effective amount of PD-L1(+) natural killer cells are administered to the patient twice per week for seven weeks.

Embodiment 50. The method of Embodiment 49, wherein administration of the PD-L1(+) natural killer cells is discontinued for 1 week; wherein another 8 week treatment cycle is optionally initiated one or more times thereafter.

Embodiment 51. The method of any one of Embodiments 13 to 24, wherein the effective amount of PD-L1(+) natural killer cells are administered to the patient twice per week for eight weeks.

Embodiment 52. The method of any one of Embodiments 13 to 51, wherein the PD-L1(+) natural killer cells are administered intravenously as an infusion for about 30 minutes to about 120 minutes.

Embodiment 53. The method of Embodiment 52, wherein the PD-L1(+) natural killer cells are administered intravenously as an infusion for about 60 minutes to about 90 minutes.

Embodiment 54. The method of any one of Embodiments 13 to 53, further comprising pretreating the patient with a low-dose and/or escalating dose of the PD-L1(+) natural killer cells prior to beginning treatment with the effective dose, wherein the low-dose and/or escalating dose is an amount lower than the effective dose.

Embodiment 55. The method of any one of Embodiments 13 to 54, further comprising administering to the patient an effective amount of a checkpoint inhibitor

Embodiment 56. The methods of Embodiment 55, wherein the checkpoint inhibitor is a PD-1 inhibitor.

Embodiment 57. The method of Embodiment 56, wherein the PD-1 inhibitor is pembrolizumab, nivolumab, cemiplimab, dostarlimab, JTX-4014, spartalizumab, camrelizumab, sintilimab, tislelizumab, toripalimab, INCMGA00012, AMP-224, or AMP-514.

Embodiment 58. The method of Embodiment 55, wherein the checkpoint inhibitor is a PD-L1 inhibitor.

Embodiment 59. The method of Embodiment 58, wherein the PD-L1 inhibitor is atezolizumab, avelumab, durvalumab, KN035, CK-301, AUNP12, CA-100570, or BMS-986189.

Embodiment 60. The method of Embodiment 58, wherein the PD-L1 inhibitor is atezolizumab.

Embodiment 61. The method of any one of Embodiments 13 to 60, wherein the cancer is non-small cell lung cancer.

Embodiment 62. The method of Embodiment 61, wherein the non-small cell lung cancer is advanced non-small cell lung cancer.

Embodiment 63. The method of Embodiment 60 or 61, wherein the non-small cell lung cancer is metastatic non-small cell lung cancer.

Embodiment 64. The method of any one of Embodiments 60 to 63, wherein the non-small cell lung cancer is recurrent non-small cell lung cancer.

Embodiment 65. The method of any one of Embodiments 13 to 60, wherein the cancer is lung cancer, neuroblastoma, glioma, myelodysplastic syndrome, B cell lymphoma, chronic myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer.

Embodiment 66. The method of any one of Embodiments 13 to 60, wherein the cancer is leukemia.

Embodiment 67. The method of Embodiment 66, wherein the leukemia is acute myeloid leukemia.

Embodiment 68. The method of any one of Embodiments 13 to 67, wherein the patient is refractory to chemotherapy.

Embodiment 69. The method of any one of Embodiments 13 to 69, wherein the patient is refractory to a PD-1 inhibitor and/or a PD-L1 inhibitor.

Embodiment 70. A nucleic acid encoding a soluble IL-15 protein, wherein the soluble IL-15 protein comprises an IL-2 signal peptide and an IL-15 protein.

Embodiment 71. The nucleic acid of Embodiment 70, wherein the nucleic acid comprises the nucleotide sequence of SEQ ID NO:1.

Embodiment 72. The nucleic acid of Embodiment 70, wherein the nucleic acid comprises the nucleotide sequence of SEQ ID NO:2.

Embodiment 73. The nucleic acid of Embodiment 70, wherein the nucleic acid comprises the nucleotide sequence of SEQ ID NO:3.

Embodiment 74. A recombinant soluble IL-15 protein, wherein the soluble IL-15 protein comprises an IL-2 signal peptide and an IL-15 protein.

Embodiment 75. The recombinant soluble IL-15 protein of Embodiment 74, wherein the soluble IL-15 protein comprises the amino acid sequence of SEQ ID NO:4.

Embodiment 76. The recombinant soluble IL-15 protein of Embodiment 74, wherein the soluble IL-15 protein comprises the amino acid sequence of SEQ ID NO:5.

Embodiment 77. The recombinant soluble IL-15 protein of Embodiment 74, wherein the soluble IL-15 protein comprises the amino acid sequence of SEQ ID NO:6.

### EXAMPLES

### Example 1

Here, we propose a phase 1 study in which CB-NK cells will be activated, expanded and genetically modified by retroviral transduction to constitutively express and secrete soluble IL-15 (sIL-15), followed by an overnight incubation with IL-18 and IL-12 to induce PD-L1 surface expression prior to cryopreservation (i.e., the COH06 product). Unmatched allogeneic COH06 will be thawed and intravenously administered following lymphocyte depletion to eligible NSCLC patients who have previously progressed on or after treatment with PD-1/PD-L1 checkpoint inhibitors. Depending on the dose level patients will receive COH06 without or with Atezo.

We have proposed a starting dose of 2×10⁸ COH06, given weekly for 4 weeks. The proposed starting dose is 25% of a single dose (i.e., 25% of −8×10⁸) of activated, unmatched allogeneic CB-NK cells constitutively expressing both membrane-bound IL-15 and a CD19 chimeric antigen receptor (CAR) at MD Anderson Cancer Center (MDACC). (2). The dose of CD19 CAR cells given to the 4 patients at MDACC without any severe adverse events (SAEs) was 1×10⁷ cells/kg which, for an average 80 kg person, is equivalent to −8×10⁸ cells. It is noteworthy that COH06 has an additional activation step via the overnight incubation in IL-12 and IL-18 that is different from the MDACC protocol, but COH06 does not include a CAR and thus is unlikely to undergo additional in vivo activation following exposure to a tumor associated antigen.

In further support of our starting dose, we have performed animal studies, infusing 1.0×10⁷ COH06 cells intravenously every other day or 2.0×10⁷ COH06 cells weekly×3 weeks without observing any toxicity in non-tumor bearing mice assessed at 28 days, and with anti-tumor efficacy in mice engrafted with the human NSCLC line A549 for 21-35 days. Assuming a multiplier of 5 when converting human lymphocytes delivered to a mouse to human lymphocytes delivered to a human per kg of body weight (as was done for conversion of CAR T dosing (13), and assuming an average body weight of 80 kg, each patient could receive a starting dose of 4.0×10⁸ COH06. However, the current study proposes to start at half this cell dose. Notably, activated PD-L1(+) NK cells that are not transduced with the sIL-15 gene would be expected to live a significantly shorter period of time in vivo (unpublished data).

### Objectives.

In adult subjects with advanced, metastatic, or recurrent non-small cell lung cancer previously treated with PD-1 and/or PD-L1 immune checkpoint inhibitors, the primary, secondary, and correlative study objectives include:

Primary Objectives. Assess the safety and determine the optimal biological dose (OBD) of COH06 as monotherapy and when give in combination with atezolizumab (Atezo).

Assess the cellular kinetics of COH06 through the detection and measurement of persistence in the peripheral blood.

Secondary Objectives. Estimate overall response (CR+PR) and disease control (CR+PR+SD) rates, including duration. Estimate the progression free survival (PFS) and overall survival (OS) rate, at 6-months and 1-year post (first) COH06 cell infusion.

Correlative Study Objectives. Assess the phenotype and activation status of COH06 via flow cytometry, PCR, and cytokine analysis. Assess T cell activation by flow cytometry and cytokine analysis.

This phase 1 trial will employ a U-BOIN (Utility Based-Bayesian Optimal Interval) design to direct the dose escalation, de-escalation, and expansion rules. The U-BOIN design consists of two seamless, connected stages. Toxicity and activity endpoints are jointly modeled using a utility function to measure dose risk-benefit trade-off; details are provided in Section 12.0. At the completion of the first stage, assuming the highest planned dose of COH06 is safe, roughly 15 patients will have been treated. At such time, an analysis of the correlative study endpoints will be completed, summarized, and provided to the FDA for review and comment. Based on the correlative study findings, clinical observations to date, and FDA feedback, patients enrolled as part of stage 2 may receive one of three possible schedules: 1) the initial COH06 cell infusion schedule as per stage 1 (one infusion per week for a total of four infusions), or 2) an increased number of COH06 cell infusions (from four to eight), administered weekly over an eight week time period, or 3) an increased number of COH06 cell infusions (from four to eight), with two infusions administered weekly over a four week period. If the combination is determined to have an acceptable safety profile during stage 1, we expect that all patients enrolled as part of stage 2 will receive Atezo.

The overall expected trial sample size is 21, assuming the highest COH06 planned dose level is well tolerated: 15 patients will be treated during stage I and 6 will be additional patients treated during stage 2. We expect to enroll approximately one to two patients per month; the expected accrual duration is 1824 months. The expected short-term follow-up period is 24 months post initial COH06 infusion. The estimated total study duration including follow-up is 48 months; 24 months from the last patient enrolled.

### Primary and Secondary Endpoints

Primary Endpoints include: Toxicity: Toxicity and adverse events will be assessed and graded according to two grading systems: 1) the NCI-Common Terminology Criteria for Adverse Events version 5.0, and 2) ASTCT Consensus Grading for Cytokine Release Syndrome (CRS) and Neurotoxicity associated with Immune Effector Cells (14), using data obtained at each clinical assessment. COH06 Cell Persistence/Activity: The magnitude and duration of persistence of COH06 cells in the peripheral blood will be quantified via flow cytometry. Biological activity is defined as any evidence of detectable COH06 cells by flow cytometric measurement on day 28.

Secondary Endpoints include: Overall Response Rate (ORR): CR/PR; Disease Control Rate (DCR): CR/PR/SD; Response duration; Disease control duration; Progression-Free Survival (PFS); and Overall Survival (OS).

Main Inclusion Criteria include: NSCLC patients with advanced, metastatic, or recurrent disease, previously treated with a PD-1 or PD-L1 immune checkpoint inhibitor, either as single agent or in combination with chemotherapy or other immunotherapy or experimental agents. Radiographically demonstrable tumor progression treatment on or after therapy with a PD-1/PD-L1 immune checkpoint inhibitor. Performance status of 0 or 1 per the ECOG scale. Preserved organ function and resolution of any prior treatment related toxicities to ≤grade 1, with the exception of alopecia and grade 2 anemia. Age ≥18 years. No cytotoxic chemotherapy or immunotherapy at any time three weeks prior to the start of lymphodepletion.

Main Exclusion Criteria include: Severe (≥grade 3) immune related adverse events during prior PD-1 or PD-L1 inhibitor treatment. Patients with EGFR mutations or ALK translocations in their tumors unless treatment with the indicated tyrosine kinase inhibitor has failed. Known positive serology for HIV. Concomitant use of other investigational agents Active brain metastases. Previously treated brain metastasis must demonstrate stability on subsequent MRI scans.

Investigational Product Dosage and Administration. The recommended dosage of atezolizumab (Atezo, TECENTRIQ®) as a single agent for NSCLC is 840 mg administered IV every 2 weeks. Atezo is supplied as 840 mg/14 mL (60 mg/mL). COH06 will be administered intravenously as a 60-90-minute infusion (≥250 mU/hour). These infusions will be carried out as outpatient procedures.

Here, we propose a first-in-human, phase 1 clinical trial in which CB-NK cells will be activated, expanded, and genetically modified by retroviral transduction to constitutively express and secrete soluble IL-15 (sIL-15). The cells have been treated with IL-18 and IL-12 to induce PD-L1 surface expression.

Prior to COH06 administration, patients will undergo lymphodepleting chemotherapy. We will utilize a standard outpatient regimen for lymphodepletion in which intravenous fludarabine 30 mg/m², cyclophosphamide 300 m g/m² and mesna 300 mg/m² are administered for a total of 3 days followed by two days of rest (2). Initiation of lymphodepleting chemotherapy will commence 5 days prior to the first infusion of COH06. The doses of the lymphodepletion regimen will be based on ideal body weight but will be adjusted if the ideal body weight is exceeded by ≥20%.

Following lymphodepletion, patients will receive 4 weekly doses of COH06 intravenously either as monotherapy or in combination with Atezo. Each patient's COH06 dose will be based on their assigned dose level. Patients who are also assigned to receive Atezo (840 mg) will receive 4 total doses administered every 14 days. One course of treatment covers an 8-week period, made up of two, 4-week cycles.

CT or MRI scans will be performed at 8 weeks in order to evaluate response to treatment. Patients experiencing progression of disease as per RECIST 1.1 will be taken off treatment. Patients experiencing stability of disease or tumor response (≥PR), may be considered for a second course of therapy (identical to their first course). The choice to not repeat lymphodepletion will be made by the trial PI in consultation with the treating physician. Patients will receive no more than two full courses (i.e. 4 months) of therapy; patients will be taken off treatment after either one or two courses of therapy. During the patient follow-up period, Atezo alone as maintenance therapy may be pursued as per the discretion of the treating physician.

Under this protocol (COH20684), patients will be followed for disease progression and survival for up to 2 years. In addition, research participants will continue with annual evaluations fora minimum of 15 years, as part of a COH Long Term follow-up protocol, in accordance with the guidance “Gene Therapy Clinical Trials—Observing Subjects for Adverse Events” for replication competent retrovirus.

### Correlative Studies Rationale

Both quantity and quality of COH06 cells are important for the potential efficacy of the treatment. We will assess the quantity and activation of NK cells over time by flow cytometric analysis and PCR. This study will use engineered NK cells and will not use CARs; thus, we do not anticipate IL-6 increases post COH06 cell administration and anticipate less toxicities as those associated with T cell engaging therapies and/or CARs. However, COH06 will be given at escalating doses and will be potentiated by the co-administration of the immune checkpoint inhibitor Atezo. To evaluate any toxicities, cytokines including GM-CSF, IFN-y, IL-2, IL-15, IL-6, IL-10, IL-12, IL-18, TNF-a, and VEGF will be measured post all blood draws. Finally, the quantity and activation of CD4 and CD8 cells will be measured to evaluate the impact of Atezo and COH06 treatment on T cells.

This phase 1 trial will employ a U-BOIN (Utility Based-Bayesian Optimal Interval) design to direct the dose escalation, de-escalation, and expansion rules. The U-BOIN design consists of two seamless, connected stages. Toxicity and activity endpoints are jointly modeled using a utility function to measure dose risk-benefit trade-off; details are provided in Section 12.0. At the completion of the first stage assuming the highest planned dose of COH06 is safe, roughly 15 patients will have been treated. At such time, an analysis of the correlative study endpoints will be completed, summarized, and provided to the FDA for review and comment. Based on the correlative study findings, clinical observations to date, and FDA feedback, patients enrolled as part of stage 2 may receive one of three possible schedules: 1) the initial COH06 cell infusion schedule as per stage 1 (one infusion per week for a total of four infusions), or 2) an increased number of COH06 cell infusions (from four to eight), administered weekly over an eight week time period, or 3) an increased number of COH06 cell infusions (from four to eight), with two infusions administered weekly over a four week period. If the combination is determined to have an acceptable safety profile during stage 1, we expect that all patients enrolled as part of stage 2 will receive Atezo.

### Treatment Course and Cycle Definition

This therapy requires only one course of treatment; the treatment course starts at lymphodepletion and goes through 8-weeks post the first COH06 infusion. A course of treatment is comprised of lymphodepletion plus two 4-week treatment cycles. The first cycle, from the first COH06 infusion to 4 weeks post (28 days), will serve as the DLT evaluation period. The second cycle, weeks 5 through 8, will start following completion of week 4. Depending on the assigned dose level, patients may also be treated concurrently with Atezo.

### Treatment Plan

### Agent Administration

Lymphodepletion. Intravenous fludarabine 30 mg/m², cyclophosphamide 300 mg/m² and mesna 300 mg/m² are administered for a total of 3 days followed by two days of rest. Initiation of lymphodepleting chemotherapy will commence 5 days prior to the first infusion of COH06. Lymphodepletion doses will be based on ideal body weight but will be adjusted if the ideal body weight is exceeded by ≥20%.

Atezolizumab (Atezo). Recommended Dosage of atezolizumab (TECENTRIQ®) for NSCLC Single Agent is 840 mg every 2 weeks or 1200 mg every 3 weeks or 1680 mg every 4 weeks administered intravenously over 60 minutes until disease progression or unacceptable toxicity. If the first infusion is tolerated, all subsequent infusions may be delivered over 30 minutes. For this protocol, we will be using 840 mg every 2 weeks recommended dose.

Atezolizumab Preparation and Administration. Visually inspect drug product for particulate matter and discoloration prior to administration, whenever solution and container permit. Discard the vial if the solution is cloudy, discolored, or visible particles are observed. Do not shake the vial. Prepare the solution for infusion as follows: Select the appropriate vial(s) based on the prescribed dose. Withdraw the required volume of Atezo from the vial(s) using sterile needle and syringe. Dilute to a final concentration between 3.2 mg/mL and 16.8 mg/mL in a polyvinyl chloride (PVC), polyethylene (PE), or polyolefin (PO) infusion bag containing 0.9% Sodium Chloride Injection, USP. Dilute with only 0.9% Sodium Chloride Injection, USP. Mix diluted solution by gentle inversion. Do not shake. Discard used or empty vials of Atezo. Storage of Infusion Solution This product does not contain a preservative. Administer immediately once prepared. If diluted Atezo infusion solution is not used immediately, store solution either: At room temperature for no more than 6 hours from the time of preparation. This includes room temperature storage of the infusion in the infusion bag and time for administration of the infusion, or under refrigeration at 2° C. to 8° C. (36° F. to 46° F.) for no more than 24 hours from time of preparation. Do not freeze. Do not shake. Administer the initial infusion over 60 minutes through an intravenous line with or without a sterile, non-pyrogenic, low-protein binding in-line filter (pore size of 0.2-0.22 micron). If the first infusion is tolerated, all subsequent infusions may be delivered over 30 minutes. Do not co-administer other drugs through the same intravenous line. Do not administer as an intravenous push or bolus.

COH06 cells Administration. The COH06 cells will be administered as a 60-90-minute infusion (≥250 mL minutes).

Active Treatment Definition. The active treatment segment of the trial will include the start of treatment (lymphodepletion) through the end of the treatment course (up to two 8-week courses), the participant is deemed intolerant to protocol therapy despite dose modification/delay, or until disease progression, death, or withdrawal of consent (whichever comes first).

### COH06; Enhanced CB-NK Cells

The cell product referred to as COH06 or Enhanced CB-NK Cells contains activated PD-L1(+) cord blood (CB) NK cells successfully transduced with a retrovirus encoding the sIL-15 gene and the tEGFR gene. The product COH06 is described in Table 1 (Release Testing of COH06). Following treatment with the retrovirus, RRV-sIL-15 tEGFR, the cells are stimulated with cytokines IL-12 and IL-18 and following expansion, cryopreserved in CryoStor® CS5 (STEMCELL®) using a controlled rate freezer. Note that the product administered to patients also contains PD-L1(+) CB-NK cells not transduced with sIL-15 or tEGFR. We refer to these cells as activated PD-L1 (+) untransduced NK cells.

Manufacture of COH06. Manufacture of the investigational NK cell product will be conducted under cGMP at the Center for Biomedicine and Genetics (CBG) located at the City of Hope. Briefly, umbilical cord blood is obtained from sources including BioIVT, Ohio State University, and StemCyte. Cord blood donors are screened in accordance with 21 CFR 1270. The NK cells are purified from cord blood using a RosetteSep Human NK Cell Enrichment Cocktail followed by centrifugation through a Ficoll-Paque gradient to develop an umbilical cord NK cell bank. The enriched NK cells are cryopreserved in CryoStor CS5; one million cells are frozen separately to be used in the assessment of the cells' proliferation capacity (17 days expansion at small scale in the presence of IL-2 and irradiated K562 feeders). NK cells that have demonstrated sufficient expansion capacity from the 1×10⁶ aliquot such that the remaining cells would be capable of generating at least 2×10⁹ cells post full-scale expansion are subsequently thawed and will be used for cell therapy productions. The NK cells will then be co-cultured with the irradiated K562 feeder cells expressing membrane-bound (IL-21) and CD-137L and exogenous IL-2. On day 7, expanded NK cells are transduced with the retroviral vector (RRV_sIL-15_tEGFR) carrying the human IL-15 gene and the truncated EGFR. Following transduction, the cells are further expanded with additional irradiated K562 feeder cells. On day 16, the cytokines IL-18 and IL-12 are added to the cell culture to harvest to upregulate endogenous expression of PD-L1 on the sIL15+ NK cells. On day 17, the cells are harvested and cryopreserved.

Handling, storage, dispensing and disposal. The COH06 cell products will be prepared by COH. Cell products are prepared using approved standard operating procedures and manufactured under cGMP conditions on COH campus. All raw materials used for the production of the cell product are released by the Office of Quality Systems (OQS) prior to use. Certificates of Analysis (CofA) for all materials referenced in any cell product batch record and all records for NK cell manipulations, including cell selection and transduction, as well as completion of batch records, labeling and tracking of the cell product will be maintained by OQS. All processes are carried out according to SOPs and include QA oversight for cGMP compliance. Cryopreserved cell products as well as prepared infusion doses are Quality Control tested as required under the Investigation New Drug Application (IND) and released for use by the Director of Quality Systems, or designee. Cryopreserved bags will be frozen using a controlled rate freezer and stored in vapor phase in a controlled access LN2 freezer until released for clinical use. The required number of cryopreserved bags will be transferred by COH OQS to the Briskin Center for eventual transfer, thaw (in a water bath) and infusion per the Investigational Brochure

Atezo is a monoclonal antibody that belongs to a class of drugs that bind to either the programmed death-receptor 1 (PD-1) or the PD-ligand 1 (PD-L1), blocking the PD-1/PD-L1 pathway, thereby removing inhibition of the immune response, potentially breaking peripheral tolerance and inducing immune-mediated adverse reactions.

Evaluation of persistence and activation of administered COH06.

Both quantity and quality of the infused NK cells are important for the potential efficacy of the treatment. We will measure NK cells in blood prior to infusion, and at several time points during the first 8 weeks of treatment (see Table 9.3.1.2 and 10.0 Study Calendar). The following tests will be performed:

Quantification: The number of COH06 cells in blood will be measured to assess the persistence of infused COH06 cells after administration via flow cytometry. NK cells are all CD56(+) CD3(−). NK cells that are transduced ex-vivo with soluble IL-15 (COH06) will express a tEGFR that is not found on non-transduced NK cells. Quantification of COH06 is as follows: WBC×% lymphocytes×% CD56(+) CD3(−) EGFR (+) or absolute lymphocyte count×% CD56(+) CD3(−) EGFR (+). A second molecular test for quantification is to perform a PCR assay on the lymphocyte fraction to assess the retroviral content (which would only be found in the COH06 population). The quantity and activation of CD4 and CD8 cells will be measured to evaluate the impact of Atezo and COH06 treatment on T cells. T cells are CD3(+) CD56(−) and will be quantified for CD4 and CD8 subsets and their expression of CD69, IFNy, granzyme B, and PD-1 via flow cytometry.

Activation: A measure of persistent activation of the COH06 can be obtained using a 4-color flow cytometric analysis on the lymphocyte gait assessing on a single cell CD56, EGFR, CD69 and PD-L1. An anti-PD-L1 antibody that reacts to a different PD-L1 epitope than Atezo, will be utilized.

Evaluation of cytokines and their association to toxicities. This study will use engineered NK cells and will not use CAR T cells, thus we do not anticipate IL-6 increases post COH06 cells administration and would anticipate lesser toxicities as those associated with T cell engaging therapies and/or CARs. However, the COH06 cells express sIL-15 will be given at escalating doses and will be potentiated by the co-administration of the immune checkpoint inhibitor Atezo. To evaluate any toxicities, blood samples will be collected to characterize cytokine levels post administration of COH06. Samples will be collected prior to the first infusion, and at several time points during the first 8 weeks of treatment. Cytokines such as GM-CSF, IFN-y, IL-2, IL-15, IL-6, IL-10, IL-12, IL-18, TNF-a, and VEGF will be measured.

Evaluation of T-cell quantity and activation. The quantity and activation of CD4 and CD8 cells will be measured to evaluate the impact of Atezo and COH06 treatment on T cells. T cells are CD3(+) CD56(−) and will be quantified for CD4 and CD8 subsets and their expression of CD69, IFNy, granzyme B, and PD-1 via flow cytometry.

COH06 Cell Persistence. The magnitude and duration of persistence of COH06 cells in the peripheral blood will be quantified via flow cytometry. NK cells are all CD56(+) CD3(−). NK cells that are transduced ex-vivo with soluble IL-15 (COH06) will express a tEGFR that is not found on non-transduced NK cells. Quantification of COH06 is as follows: WBC×% lymphocytes×% CD56(+) CD3(−) EGFR (+) or absolute lymphocyte count×% CD56(+) CD3(−) EGFR (+). A second molecular test for quantification is to perform a PCR assay on the lymphocyte fraction to assess the retroviral content (which would only be found in the COH06 population). Magnitude and duration of COH06 cell persistence will be evaluated with respect to observed toxicities and/or changes in disease status. Activity is defined as any evidence of detectable COH06 cells by flow cytometric measurement on day 28.

### Secondary Endpoint(s)

Overall Response Rate (ORR) and Disease Control Rate (DCR): Overall Response Rate (CR/PR) is calculated as the percent of evaluable subjects that have confirmed CR or PR per RECIST 1.1 criteria. Disease Control Rate (CR/PR/SD) is calculated as the percent of evaluable subjects that have confirmed CR or PR or SD per RECIST 1.1 criteria.

Evaluable for Response: Subjects will be considered evaluable for response if they are eligible, have their baseline disease assessments, receive the COH06 cell dose planned as per dose level assignment, and have their disease re-evaluated. In-evaluable subjects will not be replaced.

Response Duration: Overall Response Rate (CR/PR): Defined as the time interval from the date of first documented response (CR+PR) to documented disease relapse, progression or death whichever occurs first.

Disease Control Rate (CR/PR/SD): Defined as the time interval from the date of first documented response (CR+PR+SD) to documented disease relapse, progression or death whichever occurs first.

Progression Free Survival (PFS): Defined as time from the start of lymphodepletion to the time of disease relapse, progression, or death from any cause, whichever comes first.

Overall Survival (OS): Defined as time from the start of lymphodepletion to death from any cause.

### Study Design

This is a single-center, first-in-human phase 1 trial in which adults with advanced, metastatic or recurrent non-small cell lung cancer (previously treated with PD-1 and/or PD-L1 immune checkpoint inhibitors) will receive activated cord blood NK cells that have been genetically modified to constitutively express soluble IL-15 (COH06) as a single agent or in combination with atezolizumab. Up to five PD-L1(+) NK cell doses will be considered: 5×10⁷, 1×10⁸, 2×10⁸, 5×10⁸, and 1×10⁹ NK cells, as shown in Table 2.

TABLE 2

| Dose Level (DL) | COH06 Cell Dose | Atezolizumab Dose |
|---|---|---|
| −2 | 5 × 10⁷ | — |
| −1 | 1 × 10⁸ | — |
| 1 (starting dose) | 2 × 10⁸ | — |
| 2 | 5 × 10⁸ | — |
| 3 | 1 × 10⁹ | — |
| 4 | 1 × 10⁹ | 840 mg IV |

For each dose level, patients will be enrolled in cohorts of 3, with enrollment staggered by a minimum of two weeks for the first 3 patients treated on dose level 1. There will be no intra-patient dose escalation. Unlike early phase chemotherapy trials, in the NK cell immunotherapy trial setting, toxicity and efficacy are often not dose dependent. In addition, given the expense—in terms of patient time and effort and the cost associated with product manufacturing, an assessment of clinical activity is often necessary across all dose levels. Given this, a trial design that utilizes a dose escalation method that simply seeks to establish the maximum tolerated dose (MTD) based entirely on toxicity endpoints, may not be able to determine the optimal dose to bring forward for phase 2 investigation. For novel NK cell therapies, the objective of a dose-finding trial should be to identify the optimal biological dose (OBD), a dose that optimizes the risk-benefit (activity/toxicity) tradeoff That said, this trial will employ a utility-based Bayesian optimal interval (U-BOIN) design (15, 16) to identify the OBD.

The U-BO IN design consists of two seamless, connected stages. Toxicity and activity are jointly modeled using a multinomial-Dirichlet model and employs a utility function to measure dose risk-benefit trade-off. In stage 1, the Bayesian optimal interval (BO IN) design, with features of a 3+3 design, will be used to explore the dose space and collect preliminary toxicity and activity data to identify the admissible doses that are reasonably efficacious and safe for stage 2. In stage 2, the posterior estimate of the utility is continuously updated for each dose after each cohort, using accumulating activity and toxicity from both stages 1 and 2. The utility function then quantifies the desirability of a dose in terms of toxicity-activity tradeoff, and adaptively allocates patients to the optimal dose with the highest desirability.

To safeguard patients from toxic and/or futile doses, two dose acceptability criteria—established based on discussions between the clinical trial PI, the laboratory PI and Principal Biostatistician, are used by U-BOIN to decide which doses may be used to treat patients. A dose is deemed as admissible and eligible for treating patients if neither of the following two conditions hold: (Toxicity) Pr(TT_{T}>0.25 Idata)>0.95, (Futility) Pr(TT_{E}<0.75 data)>0.9; where TT_{T} and TT_{E} are true DLT rate and activity (NK cell persistence) rate, respectively. The DLT and activity definitions are provided in sections 7.1 and 11.1.2 of the protocol. The OBD is defined as the dose that is admissible and has the highest estimated utility.

Stage II proceeds as follows: (a) If the number of DLTs observed on the highest dose level tested is below the escalation boundary, escalate the dose for the next cohort of patients; otherwise, proceed to step b. (b) Given the observed interim data collected in both stages I and II, determine the dose utility using the utility tables. If all utilities are zero, stop the trial and no dose is selected as OBD. Repeat steps a and b until reaching the prespecified maximum sample size 21 or the number of patients treated at any dose reaches 12. When the trial is completed, select the OBD as the admissible dose that has the largest posterior mean utility.

At the completion of the first stage, assuming the highest planned dose of COH06 is safe, roughly 15 patients will have been treated. At such time, an analysis of the correlative study endpoints will be completed, summarized, and provided to the FDA for review and comment. Based on the correlative study findings, clinical observations to date, and FDA feedback, patients enrolled as part of stage 2 may receive one of three possible schedules: 1) the initial COH06 cell infusion schedule as per stage 1 (one infusion per week for a total of four infusions), or 2) an increased number of COH06 cell infusions (from four to eight), administered weekly over an eight week time period, or 3) an increased number of COH06 cell infusions (from four to eight), with two infusions administered weekly over a four week period. If the combination is determined to have an acceptable safety profile during stage 1, we expect that all patients enrolled as part of stage 2 will receive Atezo.

Tumor Imaging. As this is a phase 1 trial, measurable disease will not be required for enrollment. However, evidence of progressive disease following therapy with a PD-1 or PD-L1 inhibitor will be required. CT or MRI scans will be required to be obtained within 2 weeks of enrollment. Scans will be repeated 8 weeks following the first infusion of COH06. For patients experiencing stable disease or response, a second course of therapy, including lymphodepletion, may be considered by the principal investigator in consultation with the treating physician. At the discretion of the investigator, clinically indicated scan results will be followed every 8 weeks in responding patients for a period up to two years thereafter to document duration of response or stability, until progression is demonstrated, or the patient has been treated with a different systemic antitumor therapy.

Response and progression will be evaluated in this study using the RECIST guideline (version 1.1) (17). Changes in the largest diameter (unidimensional measurement) of the tumor lesions and the shortest diameter in the case of malignant lymph nodes are used in the RECIST criteria.

Measurable disease. Measurable lesions are defined as those that can be accurately measured in at least one dimension (longest diameter to be recorded) as >20 mm by chest x-ray, as >10 mm with CT scan, or >10 mm with calipers by clinical exam. All tumor measurements must be recorded in millimeters (or decimal fractions of centimeters). Note: Tumor lesions that are situated in a previously irradiated area might or might not be considered measurable.

Malignant lymph nodes. To be considered pathologically enlarged and measurable, a lymph node must be >15 mm in short axis when assessed by CT scan (CT scan slice thickness recommended to be no greater than 5 mm). At baseline and in follow-up, only the short axis will be measured and followed.

Non-measurable disease. All other lesions (or sites of disease), including small lesions (longest diameter <10 mm or pathological lymph nodes with ≥10 to <15 mm short axis), are considered non-measurable disease. Bone lesions, leptomeningeal disease, ascites, pleural/pericardial effusions, lymphangitis cutis/pulmonitis, inflammatory breast disease, and abdominal masses (not followed by CT or MRI), are considered as non-measurable.

Target lesions. All measurable lesions up to a maximum of 2 lesions per organ and 5 lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. A sum of the diameters (longest for non-nodal lesions, short axis for nodal lesions) for all target lesions will be calculated and reported as the baseline sum diameters. If lymph nodes are to be included in the sum, then only the short axis is added into the sum. The baseline sum diameters will be used as reference to further characterize any objective tumor regression in the measurable dimension of the disease.

Non-target lesions. All other lesions (or sites of disease) including any measurable lesions over and above the 5 target lesions should be identified as non-target lesions and should also be recorded at baseline. Measurements of these lesions are not required, but the presence, absence, or in rare cases unequivocal progression of each should be noted throughout follow-up.

Conventional CT and MRI. This guideline has defined measurability of lesions on CT scan based on the assumption that CT slice thickness is 5 mm or less. If CT scans have slice thickness greater than 5 mm, the minimum size for a measurable lesion should be twice the slice thickness. MRI is also acceptable in certain situations (e.g. for body scans). Use of MRI remains a complex issue. MRI has excellent contrast, spatial, and temporal resolution; however, there are many image acquisition variables involved in MRI, which greatly impact image quality, lesion conspicuity, and measurement. Furthermore, the availability of MRI is variable globally. As with CT, if an MRI is performed, the technical specifications of the scanning sequences used should be optimized for the evaluation of the type and site of disease. Furthermore, as with CT, the modality used at follow-up should be the same as was used at baseline and the lesions should be measured/assessed on the same pulse sequence. It is beyond the scope of the RECIST guidelines to prescribe specific MRI pulse sequence parameters for all scanners, body parts, and diseases. Ideally, the same type of scanner should be used, and the image acquisition protocol should be followed as closely as possible to prior scans. Body scans should be performed with breath-hold scanning techniques, if possible.

### Evaluation of Target Lesions

Complete Response (CR): Disappearance of all target lesions. Any pathological lymph nodes (whether target or non-target) must have reduction in short axis to <10 mm.

Partial Response (PR): At least a 30% decrease in the sum of the diameters of target lesions, taking as reference the baseline sum diameters.

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progressions).

Stable Disease (SD): Neither sufficient shrinkage to qualify for PR nor sufficient increase to qualify for PD, taking as reference the smallest sum diameters while on study.

### Evaluation of Non-Target Lesions

Complete Response (CR): Disappearance of all non-target lesions and normalization of tumor marker level. All lymph nodes must be non-pathological in size (<10 mm short axis).

Note: If tumor markers are initially above the upper normal limit, they must normalize for a patient to be considered in complete clinical response.

Non-CR/Non-PD: Persistence of one or more non-target lesion(s) and/or maintenance of tumor marker level above the normal limits.

Progressive Disease (PD): Appearance of one or more new lesions and/or unequivocal progression of existing non-target lesions. Unequivocal progression should not normally trump target lesion status. It must be representative of overall disease status change, not a single lesion increase.

Although a clear progression of “non-target” lesions only is exceptional, the opinion of the treating physician should prevail in such circumstances, and the progression status should be confirmed at a later time by the review panel (or Principal Investigator).

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria. For patients with measurable disease (i.e., target disease), the response assignment is shown in Table 3. For patients with non-measurable disease (i.e., non-target disease), the response assignment is shown in Table 4.

TABLE 3

| | | | | Best Overall |
|---|---|---|---|---|
| | | | | Response when |
| Target | Non-Target | New | Overall | Confirmation |
| Lesions | Lesions | Lesions | Response | is Required |
| CR | CR | No | CR | ≥4 weeks |
| | | | | confirmation |
| CR | Non-CR - Non-PD | No | PR | ≥4 weeks |
| CR | Not Evaluated | No | PR | confirmation |
| PR | Non-CR - Non-PD - | No | PR | |
| | Not Evaluated | | | |
| SD | Non-CR - Non-PD - | No | SD | Documents at least |
| | Not Evaluated | | | once ≥4 weeks |
| | | | | from baseline |
| PD | Any | Yes or | PD | No prior SD, PR, or |
| | | No | | CR |
| Any | PD*** | Yes or | PD | |
| | | No | | |
| Any | Any | Yes | PD | |

With reference to Table 3, patients with a global deterioration of health status requiring discontinuation of treatment without objective evidence of disease progression at that time will be reported as “symptomatic deterioration.” Every effort will be made to document the objective progression even after discontinuation of treatment.

TABLE 4

| Non-Target | New | Overall |
|---|---|---|
| Lesions | Lesions | Response |
| CR | No | CR |
| Non-CR - Non-PD | No | Non-CR - Non-PD* |
| Not all evaluated | No | Not Evaluated |
| Unequivocal PD | Yes or No | PD |
| Any | Yes | PD |

With reference to Table 4, Non-CR-Non-PD is preferred over “stable disease” for non-target disease since SD is increasingly used as an endpoint for assessment of efficacy in some trials so to assign this category when no lesions can be measured is not advised.

Duration of overall response: The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

Duration of stable disease: Stable disease is measured from the start of the treatment until the criteria for progression are met, taking as reference the smallest measurements recorded since the treatment started, including the baseline measurements.

### Example 2. Preclinical Evaluation of Off-the-Shelf PD-L1+ Natural Killer Cells Secreting IL-15 to Treat Non-Small-Cell Lung Cancer

### Introduction

Lung cancer is the leading cause of cancer-related mortality not only in the United States but also worldwide, with 85% of cases accounted for as non-small-cell lung cancer (NSCLC) (1). Immune checkpoint blockade of programmed death-1 (PD-1) and its ligand PD-L1 currently have set a new standard care for the first-line treatment of advanced NSCLC, either as monotherapy or combined with chemotherapy (2-8). Although immune checkpoint blockade (ICB) has shown impressive tumor regression in patients with advanced NSCLC, lasting responses have been limited to 15% of eligible patients (9). This high failure rate of ICB therapy results at least in part to low tumor PD-L1 expression, along with immune suppression within the tumor microenvironment (10). Thus, overall prognosis for advanced stage NSCLC remains poor.

Natural killer (NK) cells are innate lymphocytes, display cytotoxic activity against virally infected cells and tumor cells mediated in part by the recognition of the target cell and the subsequent release of cytokines and cytotoxic granules (11). They can be activated against target cells without any priming and are not restricted by major histocompatibility (MHC), yet do not mediate graft-versus-host disease (GVHD) in the setting of allogeneic stem cell transplantation (12,13), thus opening the door for off-the-shelf allogeneic NK cell therapy. In our previous study (14), we described the identification a NK cell population that upregulated PD-L1 upon recognizing tumor or upon exposure to combination of inflammatory cytokines IL-12, IL-15, IL-18, and thus referred to as tumor-reactive and cytokine-induced killer cells (TRACK NK cells). PD-L1⁺ 1 NK cells are more potent against tumor cells than PD-L1⁻ fraction. Furthermore, the administration of anti-PD-L1 antibody (atezolizumab) functions directly on PD-L1⁺ NK cells via a p38-NFκB pathway to induce the degranulation of perforin, enhanced secretion of IFN-γ, and further upregulation of PD-L1 expression via a positive feedback loop (14).

In the current study, we generate NK cells secreting soluble (s) human IL-15 and cytokine-induced expression of PD-L1 (sIL15-PDL1⁺ TRACK NK) from umbilical cord blood (UCB) NK cells. We assess the functional activity of cryopreserved sIL15-PDL1⁺ TRACK NK cells to recognize and lyse NSCLC tumor cells in vitro and in vivo, compared to non-transduced NK cells and sIL15-PDL1⁻ NK cells. Furthermore, we evaluate the safety parameters of sIL15-PDL1⁺ TRACK NK cells in the presence or absence of atezolizumab using an immunodeficient mouse model of human NSCLC.

### Methods and Materials

### Cell Lines

A549 (Cat #CCL-185) and H460 (Cat #HTB-177) cell lines were purchased from American Type Culture Collection (ATCC) and cultured in Dulbecco's Modified Eagle Medium (DMEM) supplemental with 10% fetal bovine serum (FBS) (Gibco, Cat #16000044) and 1% GlutaMax (Gibco, Cat #35050061). K562 feeder cells, which co-express 4-1BBL and membrane bound IL-21, were gifted by CytoImmune Therapeutics LLC and cultured in RPMI 1640 medium supplemented with 10% FBS.

### NK Cell Isolation and Expansion

Umbilical Cord blood (UCB) units for both research and clinical grade were obtained by StemCyte, Inc. under protocols approved by the institutional review board. UCB NK cells were incubated with RosetteSep™ Human NK Cell Enrichment Cocktail (STEMCELL, Cat #15065) and then isolated by a density-gradient centrifugation with Ficoll-Paque PLUS (Cytiva, Cat #17144003). Cryopreserved primary human NK cells were stimulated with irradiated (100 Gy) K562 feeder cells at effector I/target (T) ratio of 2:1 and recombinant human IL-2 (Proleukin, USA) in complete serum-free stem cell growth medium (SCGM) (CellGenix, Cat #20802-0500) on day 0. Activated NK cells were transduced with retroviral supernatants on day 6 in recombinant human fibronectin fragment coated plates (TakaraBio, Cat #T100B). On day 9, NK cells were stimulated again with irradiated K560 feeder cells in the presence of IL-2. On day 16, NK cells were stimulated with IL-12 (10 ng/mL) and IL-18 (10 ng/mL). On day 17, NK cells were harvested and cryopreserved in liquid nitrogen (LN2) for use in the indicated assays. All assays performed in this study were performed with cryopreserved NK cells.

### NK Cell Cytotoxicity Assay

To assess cytotoxicity, cryopreserved NK cells were thawed and co-cultured with A549 and H460 at different E/T ratios. The cytotoxicity was measured by Real-Time Cell Analysis (RTCA) as previously described (15).

### The Enzyme-Linked Immunosorbent Assay (ELISA)

The concentration of human IL-15 was measured in culture medium at the day of cell harvesting according to the manufacture's manual of the kit (R&D, Cat #S1500) with triplicates for each sample.

Cryopreserved NK cells were thawed and cultured (1×10⁶/mL) with A549 and H460 cells at E/T ratio of 5:1 in RPMI 1640 medium supplemented with 10% FBS in the absence of any cytokines for 48 hours. The concentrations of IFN-γ (R&D, Cat #DY285B-05), TNF-α (R&D, Cat #DY210-05) and granzyme B (GZMB) (R&D, Cat #DY2906-05) in the supernatants were measured according to the manufacture's manuals with triplicates for each sample.

### Flow Cytometry

For cell surface staining, cells were first resuspended in FACS buffer composed of PBS (Gibco, Cat #10010023), 2 mM EDTA (Invitrogen, Cat #15575020) and 2% FBS, followed by staining with corresponding antibodies at 4° C. for 20 minutes avoiding the light. Then cells were washed twice with FACS buffer, and subject to flow cytometric analysis via Fortessa X20 (BD Biosciences). All flow antibodies were listed in Supplementary Table 1.

### NSG Xenograft Models

NOD/SCID IL-2Ry null (NSG) xenograft model, with firefly luciferase (FFLuc)-labeled NSCLC cell lines A549 and H460, were used to assess the in vivo anti-tumor efficacy of sIL15-PDL1 NK cells. Adult NSG mice (8-12 weeks old) were purchased from the Jackson Laboratory and housed at the animal facility of City of Hope National Medical center. On day −1, NSG mice were inoculated intravenously (i.v.) with A549 or H460 cells. On day 0, according to the treatment regimens, they received an i.v. infusion of different treatments including the vehicle (PBS), NT NK cells, sIL15 NK cells or sIL15-PDL1 NK cells with or without atezolizumab intraperitoneal (i.p.) treatment. Tumor growth was monitored by bioluminescence imaging (BLI) over time. At the end point, mice were euthanized by CO₂ inhalation. Anti-coagulated blood and sera were collected for complete blood count (CBC) and chemistry testing respectively. Tissues including lung, spleen and liver were harvested and prepared as formalin-fixed, paraffin-embedded (FFPE) blocks for pathology analysis.

NSG mice also were used to evaluate the safety of sIL15-PDL1⁺ TRACK NK cells. Adult NSG mice (8-12 weeks old) were purchased from the Jackson Laboratory and housed at the animal facility of City of Hope National Medical center. On day 0, they received different treatments including the vehicle (PBS), sIL15-PDL1 NK cells (2 different donors) with or without atezolizumab treatment (i.v.). Body weight and body temperature was monitored weekly on days 7, 14, 21 and 28. At each time point of days 7, 14, and 28, mice from each group were euthanized by CO₂ inhalation. Anti-coagulated blood and sera were collected for complete blood count (CBC) and chemistry testing respectively. Human NK cells were determined as CD45⁺CD56⁺ cells in mice blood. Tissues including lung, spleen and liver were harvested and prepared as FFPE blocks for pathology analysis.

All mouse experiments were performed in accordance with protocols approved by the City of Hope Animal Care and Use Committee.

### Statistical Analysis

Student paired or unpaired t test was used to compare quantitative variables between two groups. One-way ANOVA was used for comparison among multiple groups with p values adjustment. Two-way ANOVA was used for multiple comparisons between samples with longitudinal timepoints with p values adjustment. Probability of survival was calculated using the Kaplan-Meier method. Statistical significance was assessed with the GraphPad Prism 9.0 software. P values <0.05 were considered significant.

### Results

Generation of sIL15-PDL1⁺ TRACK NK Cells

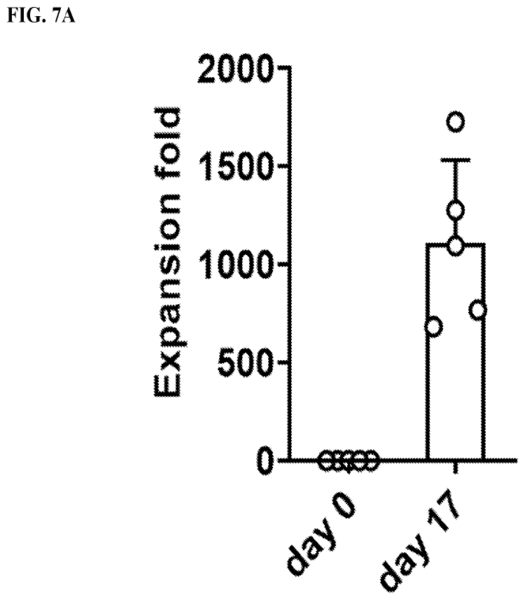

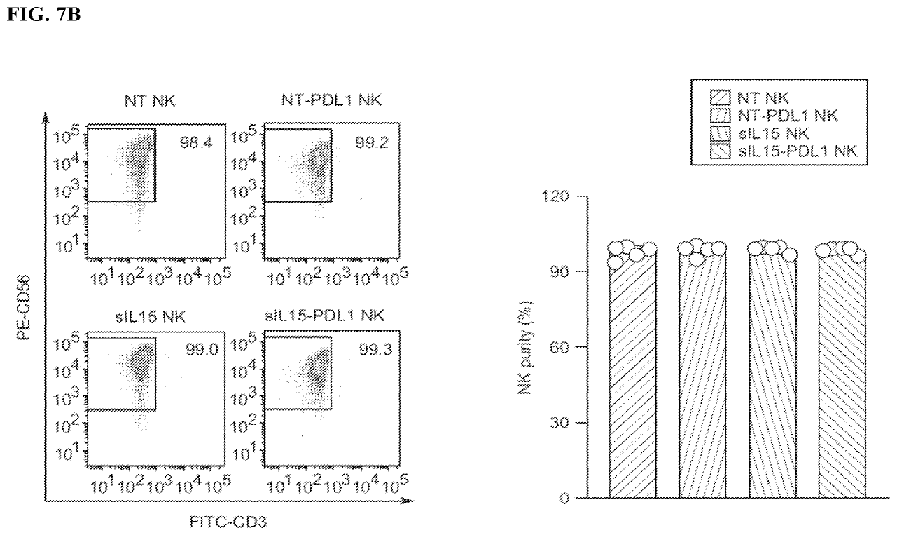

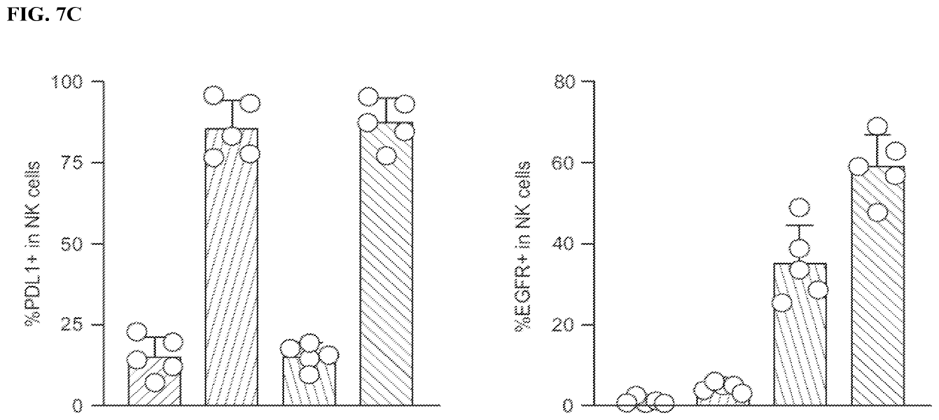

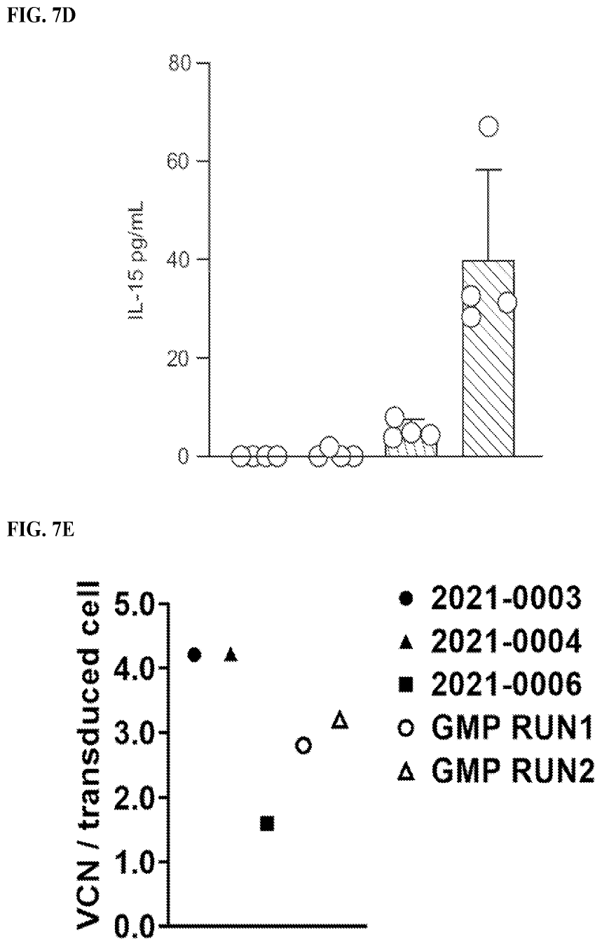

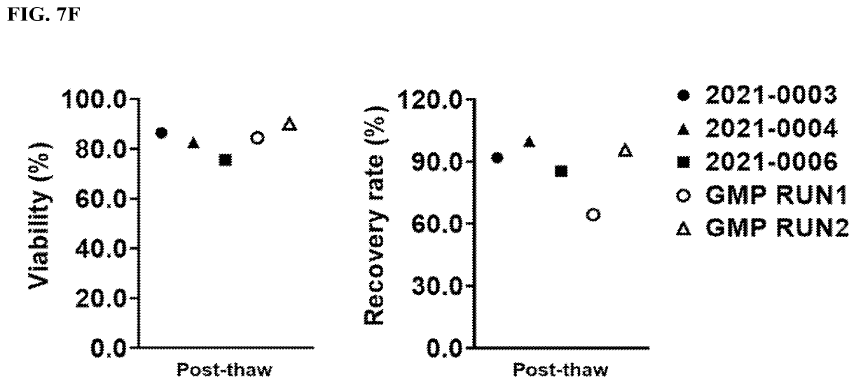

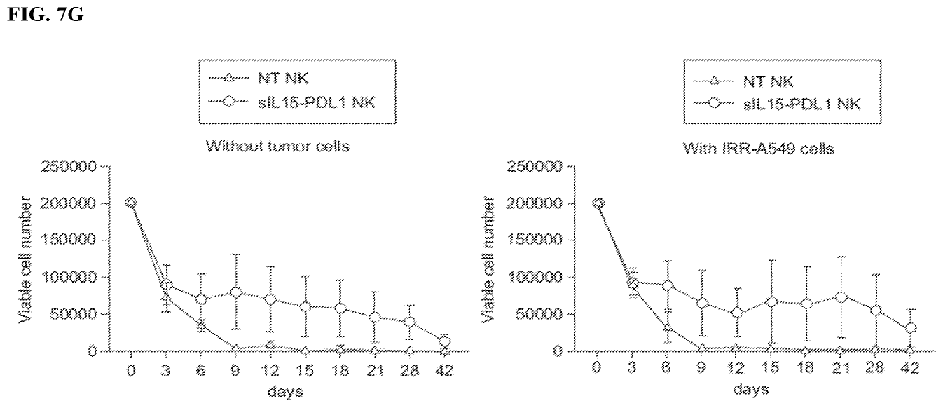

NK cells obtained from UCB were expanded with irradiated K562 feeder cells in the presence of IL-2, transduced with RVV_sIL15_tEGFR, and activated with IL-12 and IL-18 to induce the expression of PD-L1, followed by cryopreservation. As shown in FIG. 7A, sIL15-PDL1 NK cells consistently expanded over an average of 1000-fold in 17 days as did the GMP-grade NK cell product. The average NK cell purity of the sIL15-PDL1 TRACK NK⁺ cells was 98.5%, with <0.5% T cell contamination, and was similar to that in other control groups including non-transduced (NT) NK, non-transduced NK cells stimulated with IL-12 and IL-18 (NT-PDL1⁺ NK), NK cells transduced with sIL15 (sIL15 NK) but not stimulated with IL-12 and IL-18 (FIG. 7B). More than 75% of sIL15-PDL1⁺ TRACK NK cells expressed PD-L1 following overnight incubation in IL-12 and IL-18, with over 50% transduction efficiency of sIL15 as determined by the co-expression of truncated (t) EGFR (FIG. 7C). To assess the secretion of IL-15 produced by sIL15-PDL1⁺ TRACK NK cells, IL-15 concentrations were measured in the culture media supernatant on day 17. sIL15-PDL1⁺ TRACK NK cells expressed significantly higher IL-15 (mean, 39.7 pg/ml; range 28.3-66.9 pg/ml) compared to sIL15 NK cells (mean, 5.3 pg/ml; range 3.8-8.1 pg/ml), while non-transduced (NT) NK cells had undetectable levels of IL-15 regardless of PD-L1 expression (FIG. 7D). With regards to the safety of retroviral transduction, the vector copy number (VCN) of sIL15-PDL1⁺ TRACK NK cell product, which was generated with our manufacturing standard operating procedure (SOP), was assessed at <5 copies per transduced cell (FIG. 7E). Following cryopreservation, sIL15-PDL1⁺ TRACK NK cells demonstrated high recovery (mean, 87.5%) and viability (mean, 83.9%) (FIG. 7F). To investigate the possibility that the IL-15 gene in the vector may result in autonomous or dysregulated growth of NK cells, we cultured sIL15-PDL1⁺ TRACK NK cells in media without any cytokines for 42 days (n=3). Cultured sIL15-PDL1⁺ TRACK NK cells did not show any signs of abnormal growth over 6 weeks, even when co-cultured with irradiated NSCLC (A549) cells (FIG. 7G).

In Vitro Functional Activity of sIL15-PDL1⁺ TRACK NK Cells

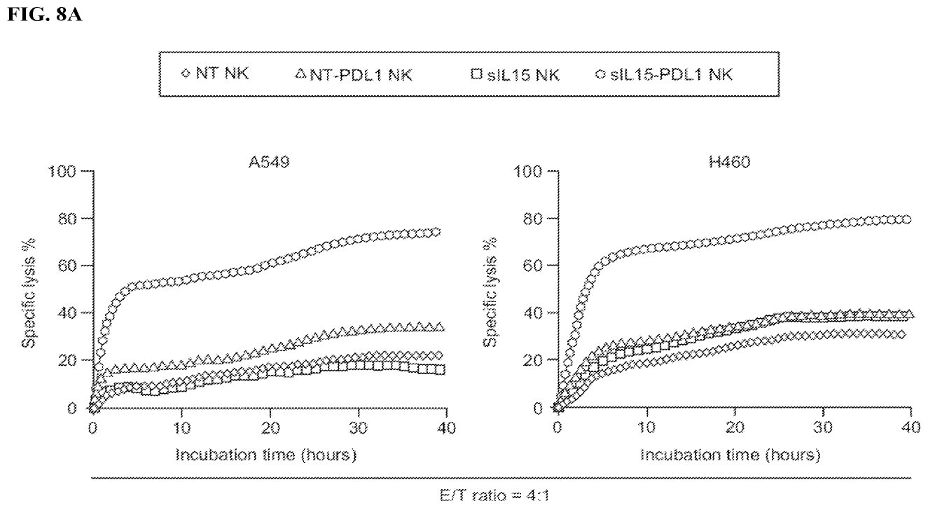

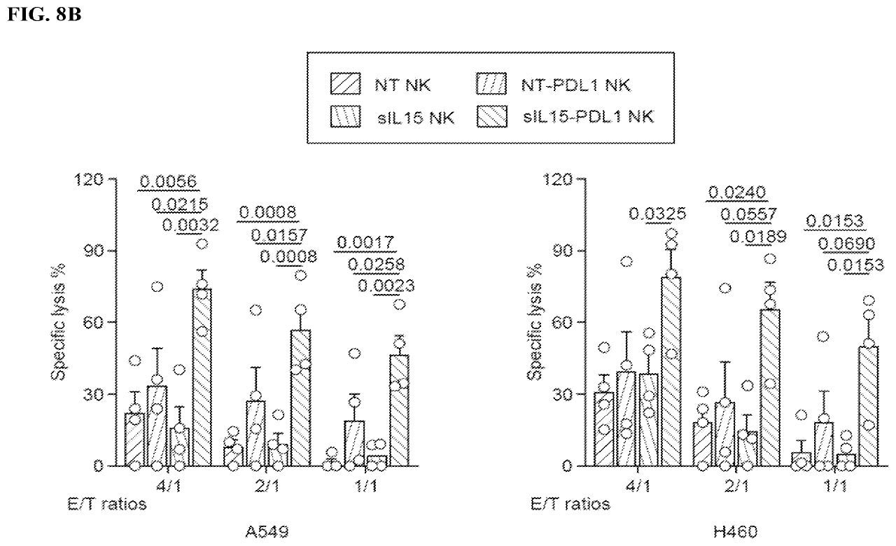

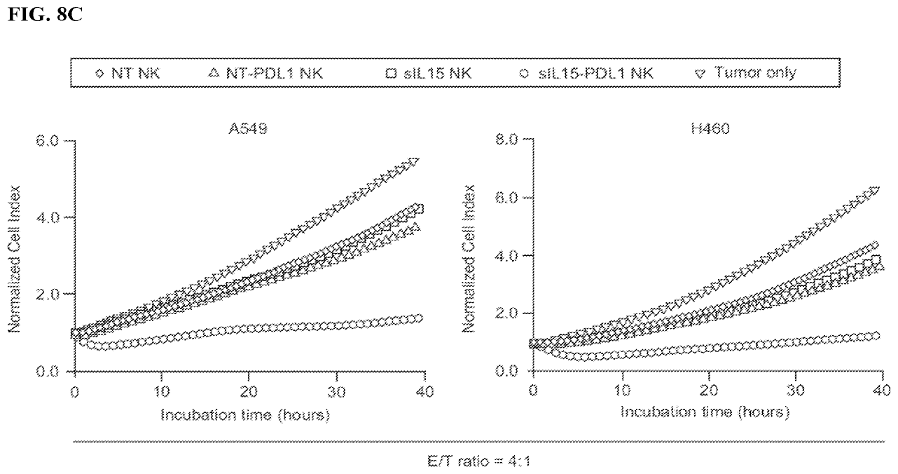

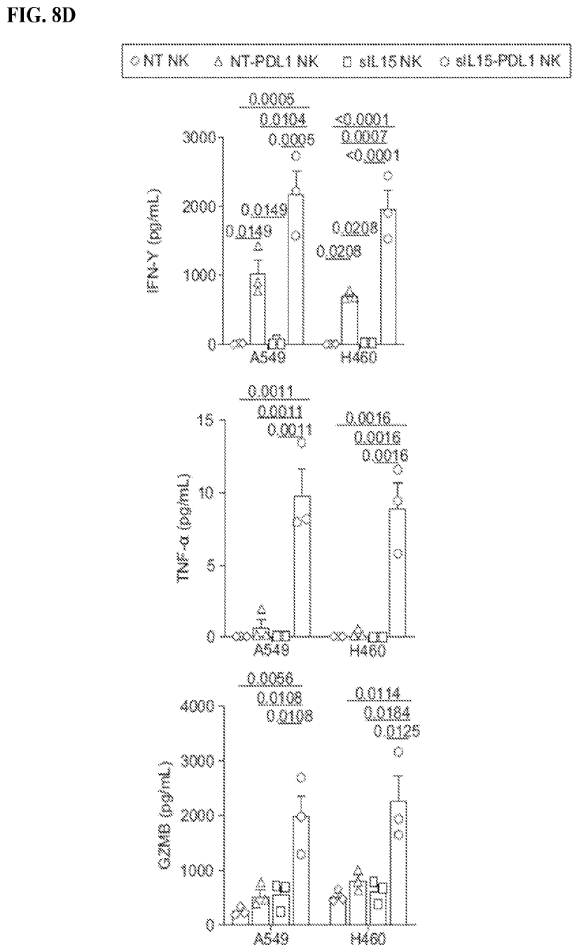

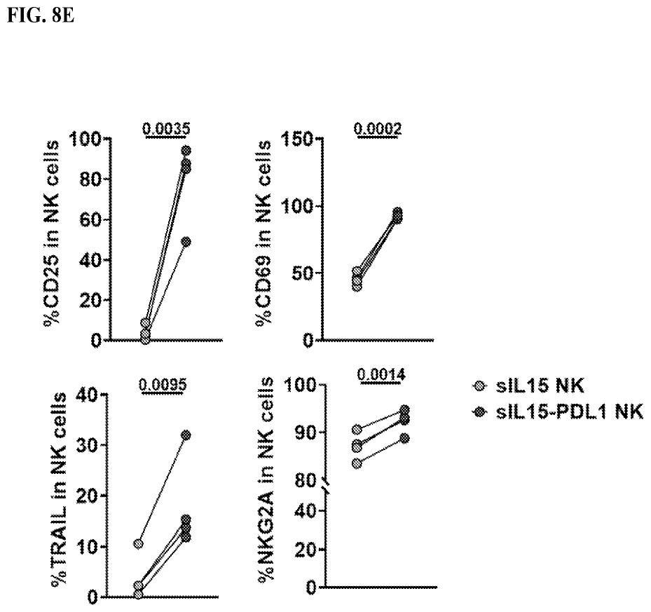

When tested against human NSCLC cell lines (A549 or H460) with Real-Time Cell Lysis Assay (RTCA) in vitro, the sIL15-PDL1⁺ TRACK NK cells lysed the NSCLC cells more efficiently than the control groups of NK cells (NT NK, NT-PDL1⁺ NK, or sIL15 NK) at different effector (E)/target (T) ratios (FIGS. 8A-8B). A549 or H460 cells, which were cocultured with sIL15-PDL1⁺ NK cells, grew significantly slower than the tumor growth seen with the control groups of NK cells (FIG. 8C). To assess the cytokine and granzyme production of sIL15-PDL1⁺ TRACK NK cells, levels of IFN-γ, TNF-α and granzyme B (GZMB) were measured in the supernatant 48 hours after NK cells were cocultured with A549 or H460 NSCLC cells. sIL15-PDL1⁺ TRACK NK cells secreted significantly higher levels of IFN-γ and TNF-α compared with NT-PDL1⁺ NK cells, while NT and sIL15 NK cells barely secreted IFN-γ and TNF-α (FIG. 8D). sIL15-PDL1⁺ TRACK NK cells consistently produced significantly higher levels of GZMB against NSCLC cells compared to all three control groups (FIG. 8D). In our previous study, we observed that the expression of CD69 and CD25 was significantly increased on PDL1⁺ NK cells compared with PDL1⁻ NK cells (14). As expected, sIL15-PDL1⁺ TRACK NK cells displayed increased expression of activation receptors, including CD69, CD25 and TRAIL, as well as inhibitory receptor NKG2A, when compared to sIL15 NK cells. However, there was no significant difference in the expression of CD16, NKG2D, NKp30, NKp44, DNAM-1, CD94 and KIR-NKAT2 between sIL15 and sIL15-PDL1⁺ TRACK NK cells. CD16 is known to shed its surface expression following activation (REF). We discovered that this shedding and resurfacing is slowed by the process of cryopreservation such that upon thawing CD16 expression is moderate, but with incubation at 37 C for 24 hours, the expression is dramatically increased, likely mimicking what occurs in vivo upon infusion of the cells. We also assessed ADCC using an anti-EGFR mAb (cetuximab) against the EGFR⁺ NSCLC cell line and showed the sIL15-PDL1⁺ TRACK NK cells lysed the NSCLC cells more efficiently than the control groups of NK cells (NT NK, NT-PDL1⁺ NK, or sIL15 NK) at different effector (E)/target (T) ratios.

In Vivo Functional Activity of sIL15-PDL1⁺ TRACK NK Cells

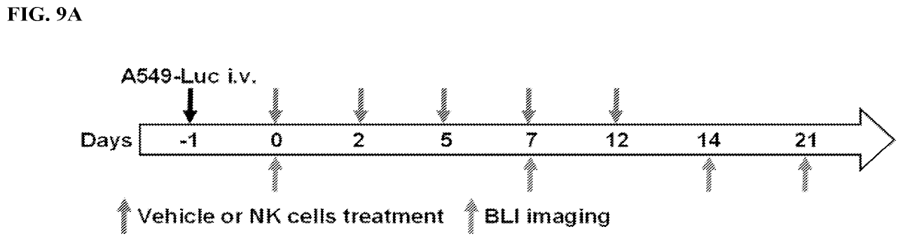

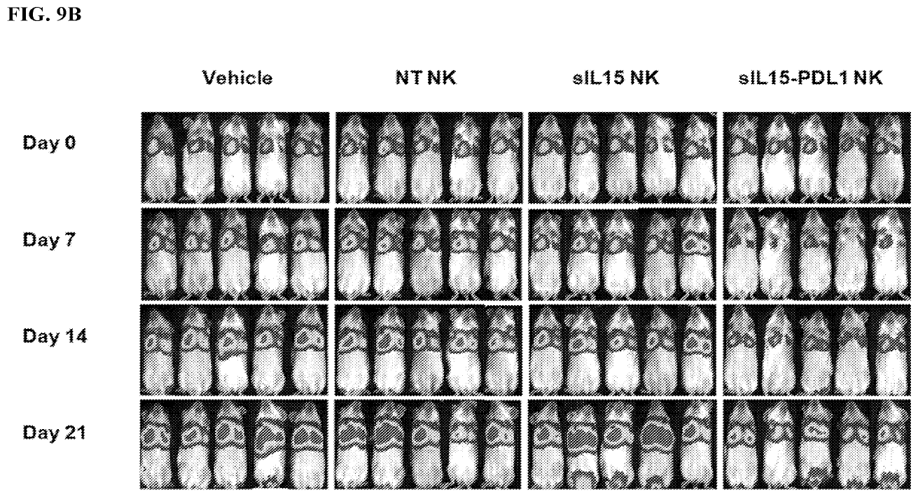

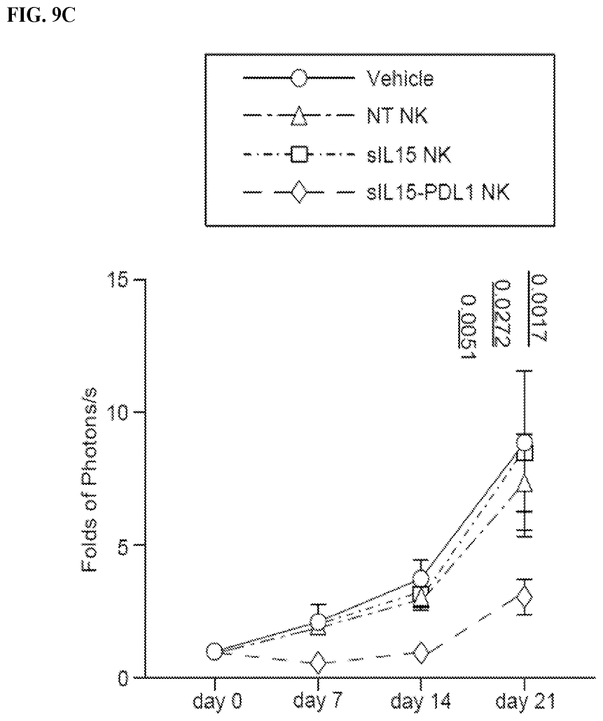

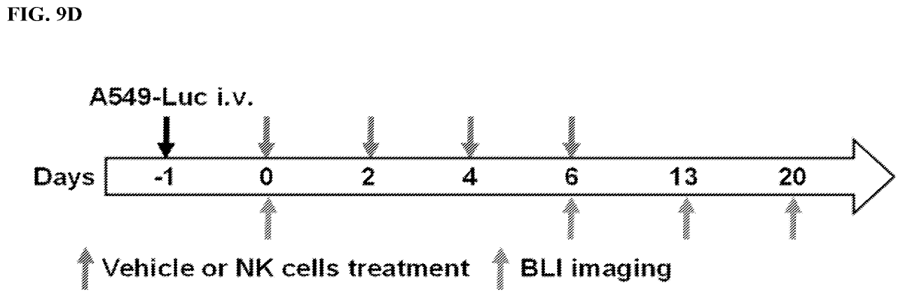

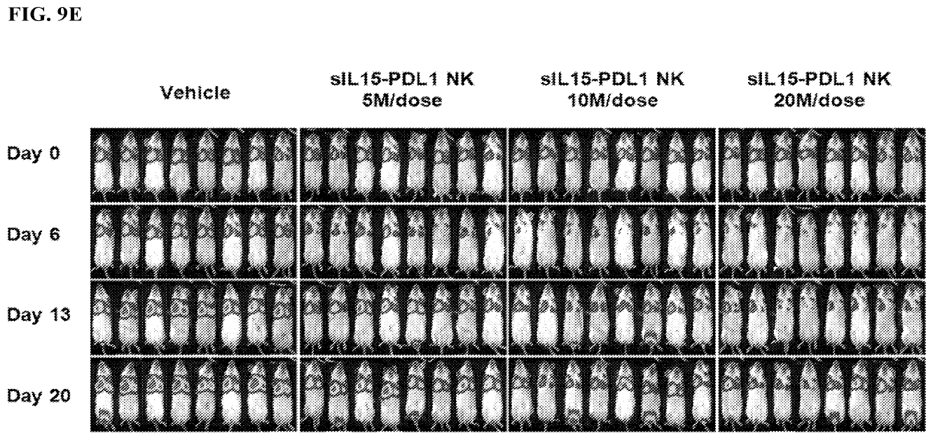

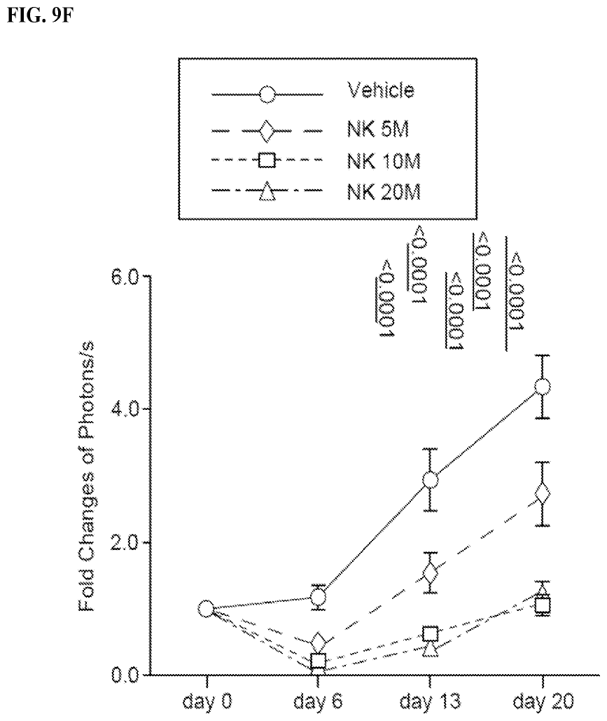

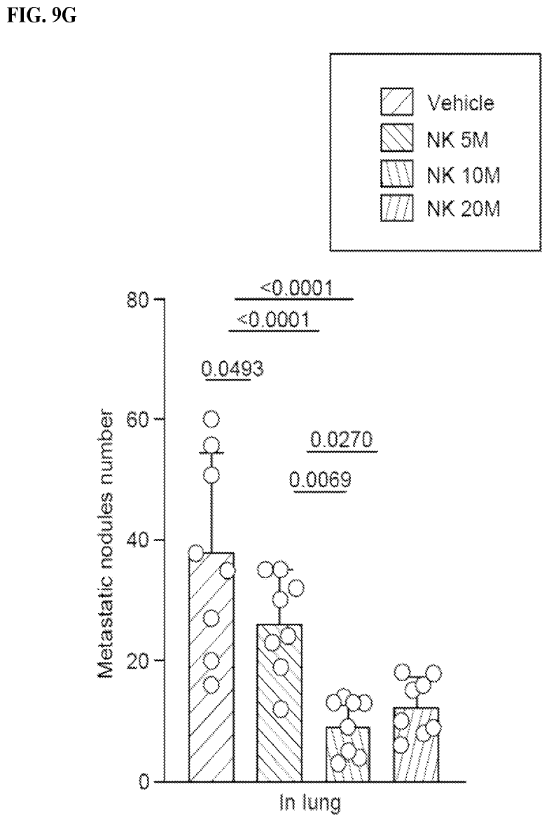

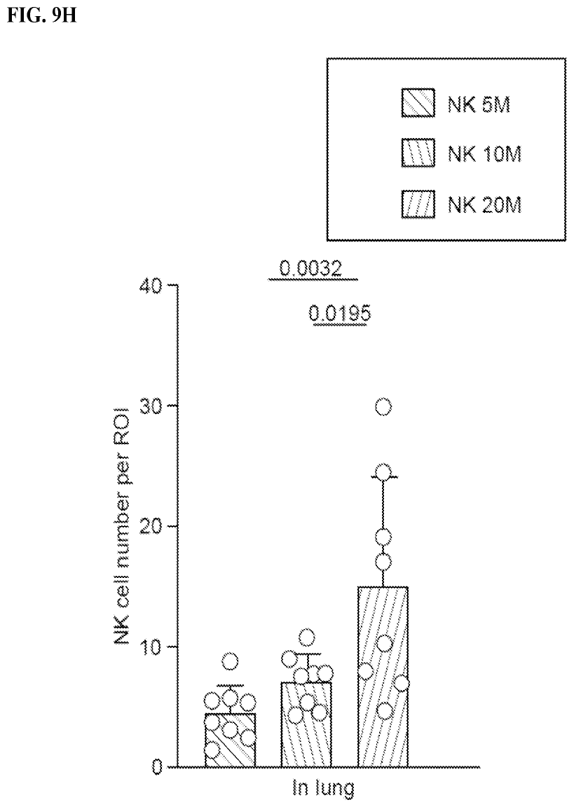

Using a NSCLC metastatic mouse model of A549 NSCLC (FIG. 9A), we next investigated whether adoptive transfer of sIL15-PDL1⁺ TRACK NK cells could control tumor progression better than the control groups of NK cells. First, mice were inoculated with A549 NSCLC cells (0.2×10⁶/mouse) on day −1, followed by five intravenous (i.v.) infusions of NK cells (10×10⁶/mouse) of either NT NK, NT-PDL1⁺ NK, sIL15 NK, or sIL15-PDL1⁺ TRACK NK cells on days 0, 2, 5, 7 and 12. The tumor burden, monitored by bioluminescence imaging (BLI), significantly decreased in the group treated with sIL15-PDL1⁺ TRACK NK cells compared to other three control groups (FIGS. 9B-9C). Next, to optimize the adoptive transfer dose of sIL15-PDL1⁺ TRACK NK cells, mice received four i.v. infusions of NK cells at three different doses (5×10⁶/mouse, 10×10⁶/mouse or 20×10⁶/mouse) under the same time schedule (FIG. 9D). We found that infusion of 10×10⁶ sIL15-PDL1⁺ TRACK NK cells provided significantly better tumor control than 5×10⁶ sIL15-PDL1⁺ TRACK NK cells, but a dose of 20×10⁶ sIL15-PDL1⁺ TRACK NK offered no better control (FIGS. 9E-9F). The number of metastatic tumor nodules in the lung decreased significantly in all NK groups compared to the group treated with vehicle only, and mice infused with 10×10⁶ or 20×10⁶ sIL15-PDL1⁺ TRACK NK cells had significantly less metastatic tumor nodules compared to those infused with 5×10⁶ sIL15-PDL1⁺ TRACK NK cells but there was no significant difference between the group infused with 10×10⁶ and 20×10⁶ sIL15-PDL1⁺ TRACK NK cells (FIG. 9G). Regarding human NK cell persistence in vivo, 20×10⁶ sIL15-PDL1⁺ TRACK NK group showed highest percentage of NK cell population in mice lung, spleen, and liver on day 21 (15 days after the final infusion) compared to mice infused with 5×10⁶ or 10×10⁶ sIL15-PDL1⁺ NK group (FIG. 9G), suggesting that in vivo, NK cells persist in tissues in a dose dependent manner.

We also investigated the safety parameters in mice on day 21, including complete blood counts (CBC), chemistry parameters and cytokine profiling. The following analyses did not have statistically significant differences in the three NK cell treatment groups compared with the vehicle control group: neutrophil (NEU), lymphocyte (LYM), red blood cell (RBC), hemoglobin (HGB) and platelet (PLT), while WBC increased significantly in mice treated with 20×10⁶ sIL15-PDL1⁺ TRACK NK cells compared to the vehicle control group. Regarding chemistry parameters, no significant increase was noted in the three NK cell treatment groups compared to the vehicle group, including alanine aminotransferase (ALT), aspartate aminotransferase (AST), alkaline phosphatase (ALP), creatine kinase (CK), albumin (ALB) and blood urea nitrogen (BUN). In the cytokine profiling analysis, the following cytokine concentrations were not statistically significantly different in three NK cell treatment groups compared to the vehicle treatment group, including human (h) IL-15, hIFNγ, hTNFα, mouse (m) IFNγ, mIL-6, and mMIP-1β.

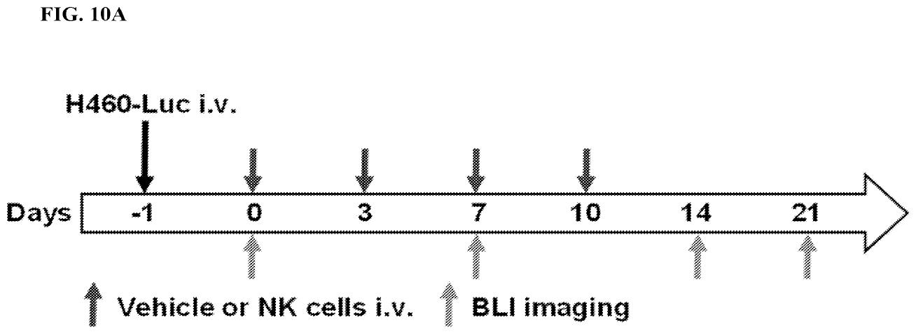

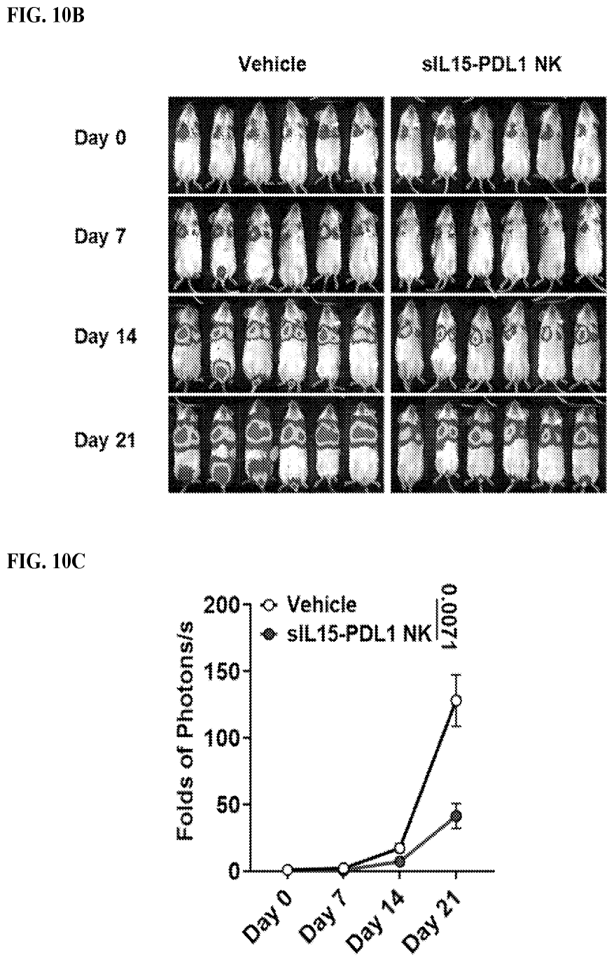

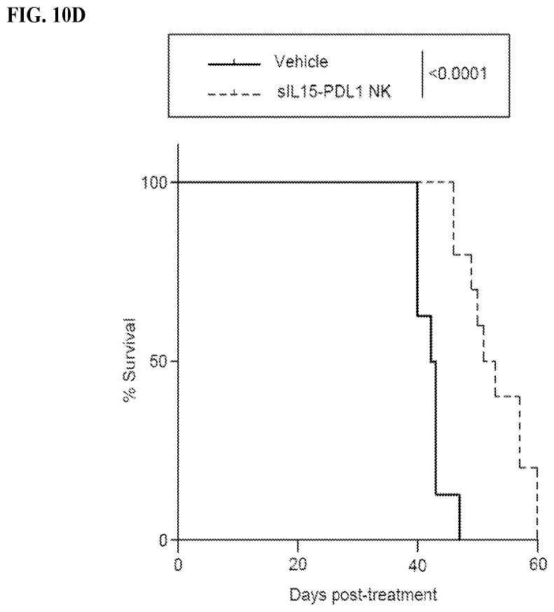

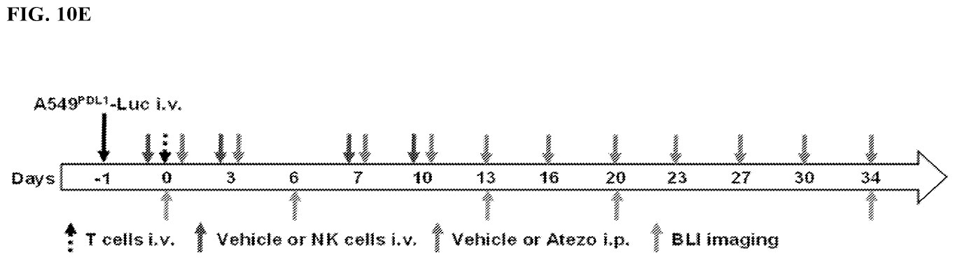

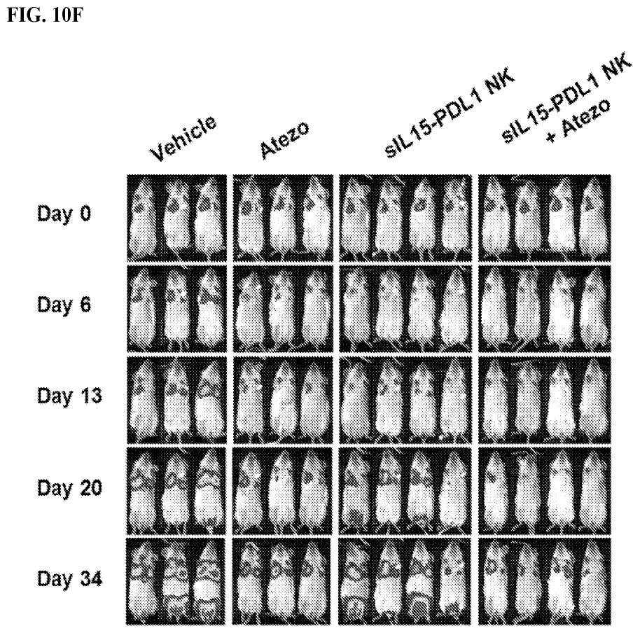

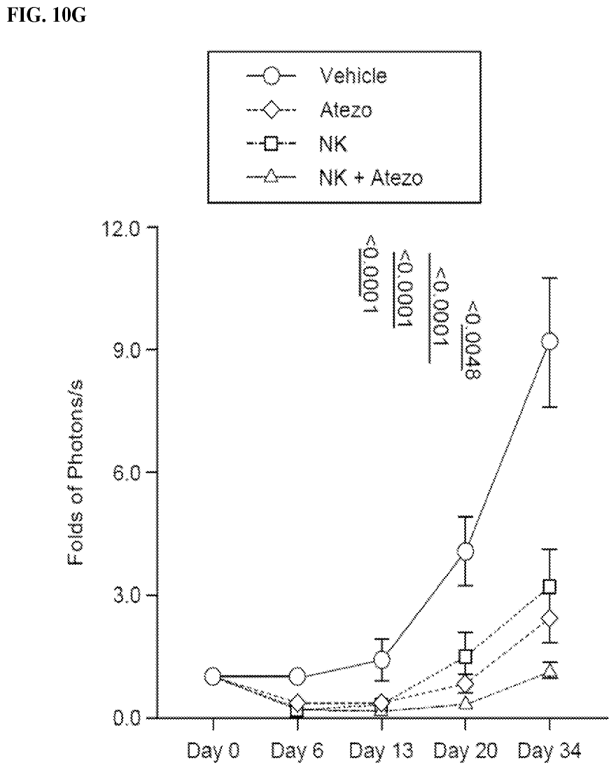

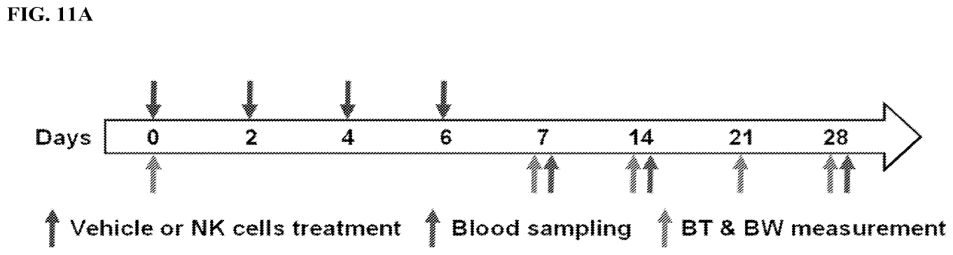

To assess sIL15-PDL1⁺ TRACK NK cells in a second NSCLC tumor model, we utilized a more aggressive metastatic mouse model with the NSCLC cell line H460 (FIG. 10A). As shown in FIG. 10B, sIL15-PDL1⁺ TRACK NK cells could suppress tumor progression better than the vehicle treated group (FIGS. 10B-10C), with a modest but significant extension of overall survival (FIG. 10D).

In Vivo Functional Activity of sIL15-PDL1⁺ TRACK NK Cells Combined with Atezolizumab

Inhibition of PD1 pathway has been used as the first-line treatment of patients with advanced NSCLC either as monotherapy or combined with chemotherapy (7). The anti-PD-L1 antibody atezolizumab has been shown to provide an overall survival benefit in patients with previously treated metastatic NSCLC regardless of PD-L1 expression (16-18). We previously demonstrated the atezolizumab activates PD-L1⁺ TRACK NK cells via a p38-NFκB pathway (14). To assess whether the addition atezolizumab can further increase the anti-tumor efficacy of the sIL15-PDL1⁺ TRACK NK cells, we treated A549-engrafted mice with sIL15-PDL1⁺ TRACK NK cells in the absence of presence of atezolizumab (FIG. 10E). Among all the treatments, the group of sIL15-PDL1⁺ TRACK NK cells in the presence of atezolizumab showed the best suppression of tumor growth compared to other three treatment groups (FIGS. 10F-10G).

Safety Evaluation of sIL15-PDL1 NK Cells

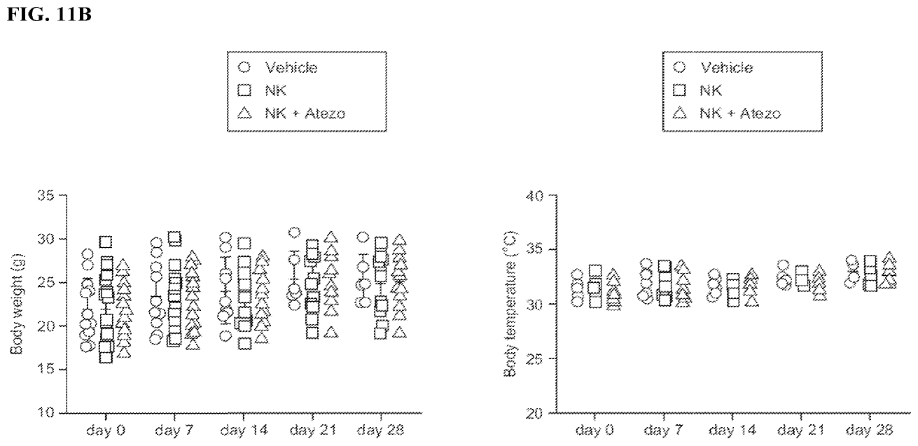

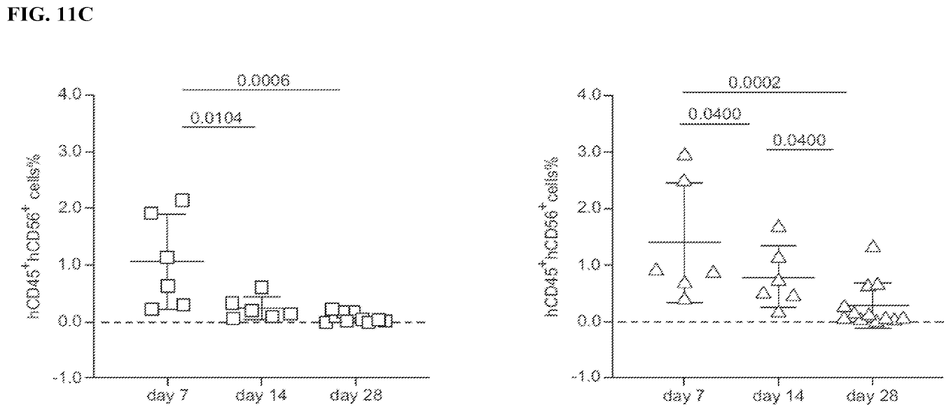

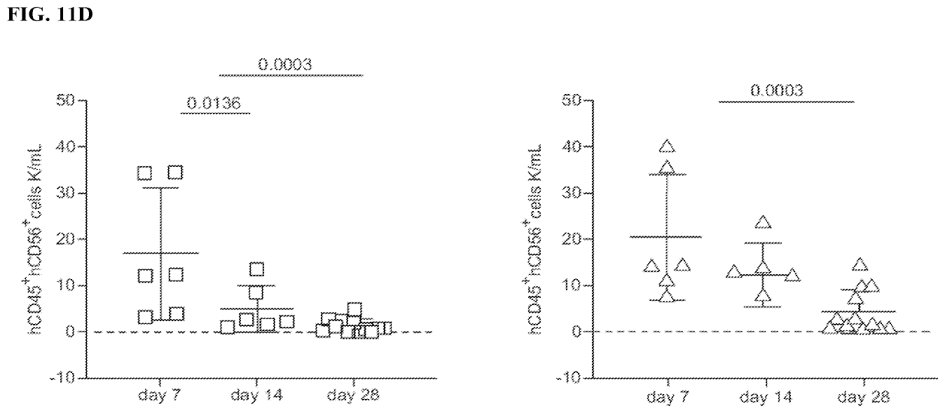

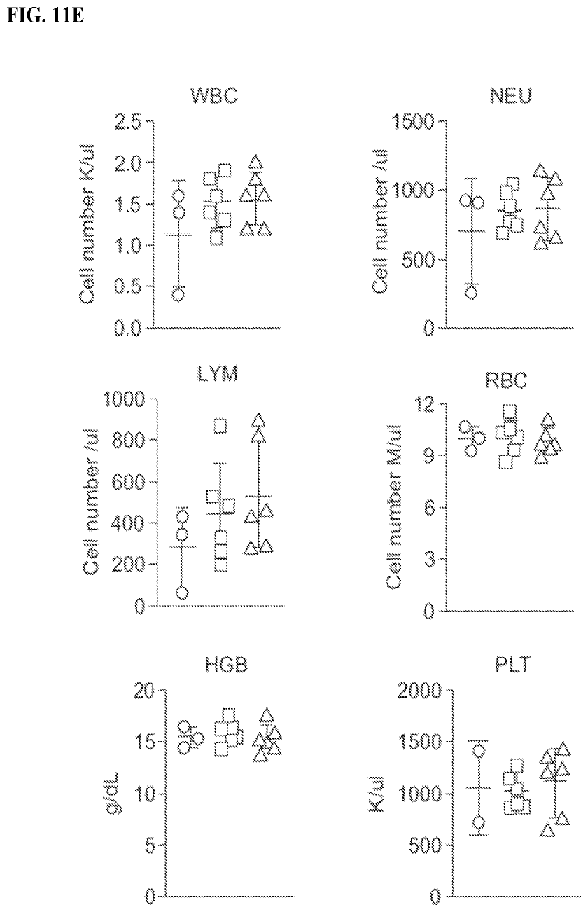

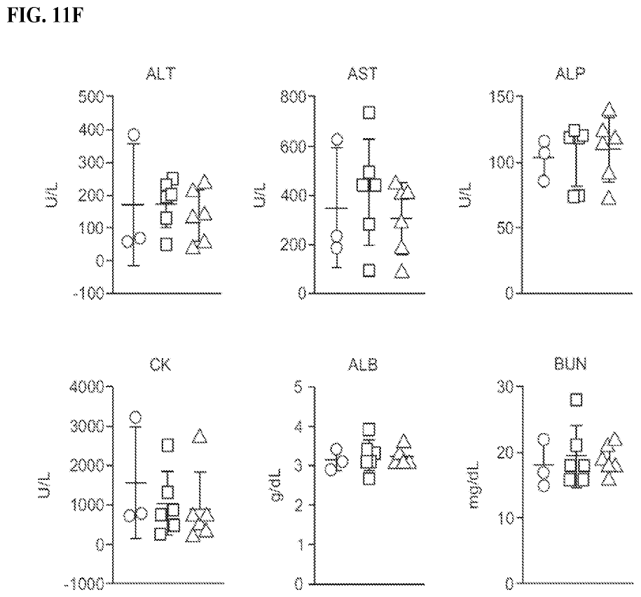

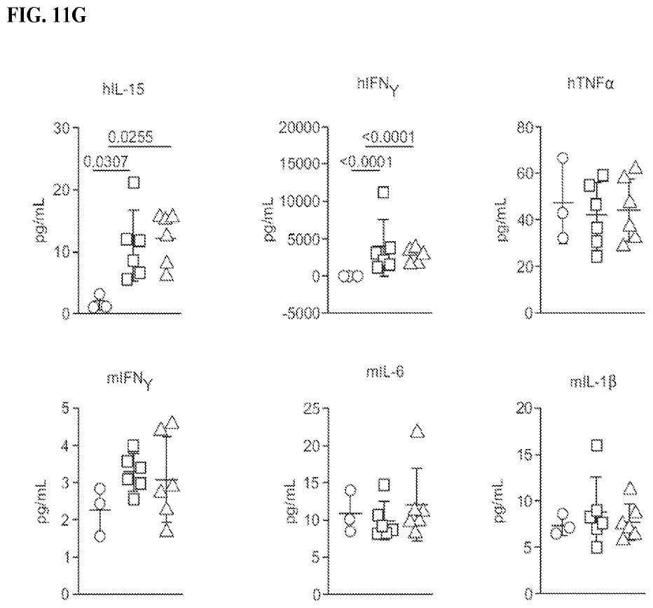

Finally, the safety evaluation of sIL15-PDL1 NK cell has been performed with a total of 60 mice (30 females, 30 males) divided into three groups: the vehicle, sIL15-PDL1⁺ TRACK NK cells and sIL15-PDL1⁺ TRACK NK cells with atezolizumab (atezo). Each group treated with sIL15-PDL1⁺ TRACK NK cells included two different donors, one of which was a GMP-grade NK cell product. No NSCLC tumors were engrafted for these safety studies. Different groups of mice were euthanized at different time points: early time point (day 7), middle time point (day 14) and end time point (day 28). CBC, chemistry parameters, cytokines profiling, and histopathology was evaluated at each time point. During the whole study, mice body weight and body temperature were monitored every week (FIG. 11A).

Overall, as shown in FIG. 11B, body weight and body temperature did not show any significant difference among the groups of the vehicle, sIL15-PDL1⁺ TRACK NK cells in the III absence or presence of atezolizumab treatment. While assessing sIL15-PDL1⁺ TRACK NK cell persistence in vivo, our data showed highest percentage of hCD45⁺hCD56⁺ population in mice blood on day 7 (24 hours post all NK cell infusions) when compared to those on day 14 (8 days post all NK cell infusions), and day 28 (22 days post all NK cell infusions) (FIG. 11C), as well as the absolute numbers of hCD45⁺hCD56⁺ cells in 1 mL mice blood (FIG. 11D), indicating sIL15-PDL1⁺ TRACK NK cells are not proliferating autonomously in vivo, consistent with in vitro results noted earlier (FIG. 7G).

We also assessed CBCs, blood chemistries and serum cytokine levels at different time points to provide more information about the safety of our sIL15-PDL1⁺ TRACK NK cell product in the absence or presence of atezolizumab. At the earliest time point of day 7 in which the human NK cells population was the highest in mice, white blood cell lineages (WBC, NEU and LYM), red blood cell parameters (RBC, HGB) and platelets did not show any differences among the groups of the vehicle, sIL15-PDL1⁺ TRACK NK cells in the absence or presence of atezolizumab (FIG. 11E). Similar findings were seen in liver function parameters (ALT, AST, ALP) and kidney function parameters (CK, ALB, BUN) (FIG. 11F). Regarding the cytokine profiling assessment, the following analyses did not show statistically significant differences among all three groups: hTNFα, mIFNγ, mIL-6 and mIL-10, while concentrations of hIL-15 and hIFNγ were significantly higher in groups treated with sIL15-PDL1⁺ TRACK NK cells regardless of the presence or absence of atezolizumab and compared to those in the vehicle group (FIG. 11G). Furthermore, we did not observe any significant difference in CBC, chemistry, and cytokine profiling testing either at the middle time point of day 14, or the end time point of day 28. Similarly, there was no evidence of tissue damage including lung, liver, and spleen in mice post s sIL15-PDL1⁺ TRACK NK cell infusion in the absence or presence of atezolizumab treatment at the end time point day 28. Altogether, these data demonstrate that the administration of sIL15-PDL1⁺ TRACK NK cells with or without atezolizumab is safe and does not induce toxicity.

### DISCUSSION

The expression of PD-L1 not only has been extensively reported on tumor cells, but the expression of PD-L1 on immune cells has also been revealed on macrophages (19), T cells (20), and NK cells (21). In our previous study, we found PD-L1 expression on primary human NK cells after encountering tumor cells or stimulated with cytokines (14). We also observed that CD69 and CD25 were significantly increased on PD-L1⁺ NK cells compared to PD-L1⁻ NK cells, whereas the expression of CD94, KLRG1, NKp44, and NKG2D did not show an obviously difference in these two subsets of NK cells. Consistently, in current study, sIL15-PDL1 NK cells, which were stimulated with IL-12/18 before harvesting, expressed significantly higher NKG2A, CD25, CD69 and TRAIL with unchanged NKp30, NKp44, NKG2D, KIR-NKAT2, DNAM-1 and CD16 when compared with sIL15 NK cells.

NK cells are classically referred to as innate cells, but they also share many properties with other adaptive lymphocytes, particularly CD8⁺ T cells, including a common lymphoid progenitor, common surface markers, secretion of perforin and granzymes to mediate cytotoxicity (22,23). Recently, many studies described “memory-like” properties of NK cells, an extended shared attributes with T cells, with providing evidence for virus-induced memory NK cells, cytokine-induced memory NK cells, and liver-restricted memory NK cells (24). Since NK cells do express receptors to response to many cytokines (e.g., IL-2, IL-12, IL-15, IL-18 etc.), Yokoyama and colleagues found that pre-activating mouse NK cells with IL-12, IL-15 and IL-18, which they defined as memory-like NK cells, exhibited enhanced responses to restimulation with cytokines after adoptive transfer into naïve Rag1^{-/-} mice compared to IL-15 activating-transferred cells (25). This pre-activation strategy also resulted in enhanced mouse NK cells accumulated and persisted in the tumor tissue with potent effector function (26). Similarly, human IL-12, IL-15, and IL-18-induced memory-like NK cells have better expansion and anti-tumor ability in response to exogenous IL-2 after adoptive transfer into immunodeficient mice (27). Regarding the phenotypic changes, memory-like NK cells express upregulated inhibitory or activating receptors including NKG2A, NKp30, NKp44, NKp46, NKG2D, CD62L, and CD25, whereas other receptors appear unchanged such as KIR, CD57, NKG2C, DNAM-1, CD137, and CD11b or decreased (e.g., NKp80) (28). Taken these together, it indicates that our sIL15-PDL1 NK cells having partially memory-like properties.

Our prior work has demonstrated ex vivo expanded human NK cells without any cytokines could not survival a long time in vivo, whereas engineered NK cells with soluble IL-15 integration had much better persistence in vivo (15). Similarly, high frequencies of CAR NK cells were identified in blood, bone marrow, liver and spleen of mice treated with anti-CD19 CAR NK cells with IL-15 expression, while CAR NK cells' persistence and homing in mice treated with conventional anti-CD19 CAR NK cells was more limited (29). Some studies have shown IL-15 causing considerable toxicity in nonhuman primates at higher doses, including weight loss and skin rash (30-32). In another study regarding IL-15 superagonist which was composed with IL-15 and soluble IL-15Rα, Guo et al. found IL-15 superagonist mediated systemic physiologic dysfunction reflected by the development of hypothermia, weight loss and acute liver injury in mice which could be partially reversed by NK cell depletion (33). Conlon et al. showed that IL-15 treatment could clear pulmonary lesions in patients with malignant melanoma, but also with some side effects including liver injury, fever, and thrombocytopenia (34). In a Phase I clinical trial of continuous intravenous infusion of rhIL-15 to treat solid tumors, eight of twenty-seven adult patients had serious adverse events and two patients died (35). Moreover, this year Christodoulou and the colleagues in this year reported that IL-15 secreting CAR-NK cells caused early mortality in mice engrafted with MV-4-11 cells, which was associated with high numbers of infiltrating NK cell, increasing levels of systemic soluble IL-15 and high levels of human TNF-α (36). However, we evaluated the safety of sIL15-PDL1 NK cells in mice with or without tumor engraftment. The frequencies and absolute numbers of sIL15-PDL1 NK cells were decreased gradually post infusion in mice no matter tumor engrafted or not. We also did not observe any significant changes in parameters of body weight, body temperature, complete blood count, liver function, kidney function, and cytokine profile, suggesting our product having low possibility to cause toxicity after moved forward to the clinic.

Nowadays cellular immunotherapy of NK cells has reached promising avenues of hematologic malignancies treatment, but so far these successes have not been successfully transferred to the treatment of solid tumors. One of reasons causing this challenge is that NK cells are hard to migrate into tumor beds and penetrate the barriers imposed by solid tumors (37). In our study, the results of immunohistochemistry staining analysis showed sIL15-PDL1 NK cells detectable in mice lung after two weeks post all infusions with a dose dependent manner, as well as in mice spleen and liver, suggesting sIL15-PDL1 NK cells having trafficking ability to solid tumor sites. Our previous work showed anti-PD-L1 mAb (atezolizumab) augmenting human PD-L1⁺ NK cells antitumor activity in vivo. Besides the addition of atezolizumab treatment could enhanced sIL15-PDL1 NK cells' tumor suppression ability in mice engrafted either with A549 or H460, we also noticed that there were more sIL15-PDL1 NK cells trafficking into mice lung, liver, and spleen in the presence of atezolizumab compared to that in the absence of atezolizumab (data not shown). By using the murine colon adenocarcinoma (MC38) and murine lymphoma (A20) models, Tang et al. observed that anti-PD-L1 antibody could be accumulated in tumor tissues, regardless of the status of PD-L1 expression on tumor cells (38). This probably could explain what we found in our current study, indicating the trafficking ability of sIL15-PDL1 NK cells in the presence of atezolizumab.

In summary, our in vitro and in vivo studies using human allogeneic off-the-shelf sIL15-PDL1 NK cells demonstrated enhanced cytotoxicity against NSCLC in vitro and enhanced control of tumor growth in vivo, further improved with atezolizumab. Moreover, the intravenous infusion of sIL15-PDL1 NK cells naturally traffic to lung in vivo. All those promising results will support our sIL15-PDL1 NK cells platform moved forward to the clinic for the treatment of relapses and refractory non-small cell lung cancer patients.

### REFERENCES

1. Herbst R S, Heymach J V, Lippman S M. Lung cancer. N Engl J Med 2008; 359(13):1367-80 doi 10.1056/NEJMra0802714.
2. Socinski M A, Jotte R M, Cappuzzo F, Orlandi F, Stroyakovskiy D, Nogami N, et al. Atezolizumab for First-Line Treatment of Metastatic Nonsquamous NSCLC. N Engl J Med 2018; 378(24):2288-301 doi 10.1056/NEJMoa1716948.
3. Reck M, Mok TSK, Nishio M, Jotte R M, Cappuzzo F, Orlandi F, et al. Atezolizumab plus bevacizumab and chemotherapy in non-small-cell lung cancer (IMpower150): key subgroup analyses of patients with EGFR mutations or baseline liver metastases in a randomised, open-label phase 3 trial. Lancet Respir Med 2019; 7(5):387-401 doi 10.1016/S2213-2600(19)30084-0.
4. Gandhi L, Rodriguez-Abreu D, Gadgeel S, Esteban E, Felip E, De Angelis F, et al. Pembrolizumab plus Chemotherapy in Metastatic Non-Small-Cell Lung Cancer. N Engl J Med 2018; 378(22):2078-92 doi 10.1056/NEJMoa1801005.
5. Paz-Ares L, Luft A, Vicente D, Tafreshi A, Gumus M, Mazieres J, et al. Pembrolizumab plus Chemotherapy for Squamous Non-Small-Cell Lung Cancer. N Engl J Med 2018; 379(21):2040-51 doi 10.1056/NEJMoa1810865.
6. West H, McCleod M, Hussein M, Morabito A, Rittmeyer A, Conter H J, et al. Atezolizumab in combination with carboplatin plus nab-paclitaxel chemotherapy compared with chemotherapy alone as first-line treatment for metastatic non-squamous non-small-cell lung cancer (IMpower130): a multicentre, randomised, open-label, phase 3 trial. Lancet Oncol 2019; 20(7):924-37 doi 10.1016/S1470-2045(19)30167-6.
7. Herbst R S, Giaccone G, de Marinis F, Reinmuth N, Vergnenegre A, Barrios C H, et al. Atezolizumab for First-Line Treatment of PD-L1-Selected Patients with NSCLC. N Engl J Med 2020; 383(14):1328-39 doi 10.1056/NEJMoa1917346.
8. Akinboro O, Larkins E, Pai-Scherf L H, Mathieu L N, Ren Y, Cheng J, et al. FDA Approval Summary: Pembrolizumab, Atezolizumab, and Cemiplimab-rwlc as single agents for first-line treatment of advanced/metastatic PD-L1 high NSCLC. Clin Cancer Res 2022 doi 10.1158/1078-0432.CCR-21-3844.
9. Yu Y, Zeng D, Ou Q, Liu S, Li A, Chen Y, et al. Association of Survival and Immune-Related Biomarkers With Immunotherapy in Patients With Non-Small Cell Lung Cancer: A Meta-analysis and Individual Patient-Level Analysis. JAIA Netw Open 2019; 2(7):e196879 doi 10.1001/jamanetworkopen.2019.6879.
10. Chang C H, Qiu J, O'Sullivan D, Buck M D, Noguchi T, Curtis J D, et al. Metabolic Competition in the Tumor Microenvironment Is a Driver of Cancer Progression. Cell 2015; 162(6):1229-41 doi 10.1016/j.cell.2015.08.016.
11. Vivier E, Raulet D H, Moretta A, Caligiuri M A, Zitvogel L, Lanier L L, et al. Innate or adaptive immunity? The example of natural killer cells. Science 2011; 331(6013):44-9 doi 10.1126/science.1198687.
12. Ruggeri L, Capanni M, Urbani E, Perruccio K, Shlomchik W D, Tosti A, et al. Effectiveness of donor natural killer cell alloreactivity in mismatched hematopoietic transplants. Science 2002; 295(5562):2097-100 doi 10.1126/science.1068440.
13. Asai O, Longo D L, Tian Z G, Hornung R L, Taub D D, Ruscetti F W, et al. Suppression of graft-versus-host disease and amplification of graft-versus-tumor effects by activated natural killer cells after allogeneic bone marrow transplantation. J Clin Invest 1998; 101(9):1835-42 doi 10.1172/JC11268.
14. Dong W, Wu X, Ma S, Wang Y, Nalin A P, Zhu Z, et al. The Mechanism of Anti-PD-L1 Antibody Efficacy against PD-L1-Negative Tumors Identifies NK Cells Expressing PD-L1 as a Cytolytic Effector. Cancer Discov 2019; 9(10):1422-37 doi 10.1158/2159-8290.CD-18-1259.
15. Lu T, Ma R, Dong W, Teng K Y, Kollath D S, Li Z, et al. Off-the-shelf CAR natural killer cells secreting IL-15 target spike in treating COVID-19. Nat Commun 2022; 13(1):2576 doi 10.1038/s41467-022-30216-8.
16. Chen D S, Irving B A, Hodi F S. Molecular pathways: next-generation immunotherapy-inhibiting programmed death-ligand 1 and programmed death-1. Clin Cancer Res 2012; 18(24):6580-7 doi 10.1158/1078-0432.CCR-12-1362.
17. Herbst R S, Soria J C, Kowanetz M, Fine G D, Hamid O, Gordon M vS, et al. Predictive correlates of response to the anti-PD-L1 antibody MPDL3280A in cancer patients. Nature 2014; 515(7528):563-7 doi 10.1038/nature14011.
18. Rittmeyer A, Barlesi F, Waterkamp D, Park K, Ciardiello F, von Pawel J, et al. Atezolizumab versus docetaxel in patients with previously treated non-small-cell lung cancer (OAK): a phase 3, open-label, multicentre randomised controlled trial. Lancet 2017; 389(10066):255-65 doi 10.1016/50140-6736(16)32517-X.
19. Hartley G P, Chow L, Ammons D T, Wheat W H, Dow S W. Programmed Cell Death Ligand 1 (PD-L1) Signaling Regulates Macrophage Proliferation and Activation. Cancer Immunol Res 2018; 6(10):1260-73 doi 10.1158/2326-6066.CIR-17-0537.
20. Latchman Y E, Liang S C, Wu Y, Chernova T, Sobel R A, Klemm M, et al. PD-L1-deficient mice show that PD-L1 on T cells, antigen-presenting cells, and host tissues negatively regulates T cells. Proc Natl Acad Sci USA 2004; 101(29):10691-6 doi 10.1073/pnas.0307252101.
21. Terme M, Ullrich E, Aymeric L, Meinhardt K, Coudert J D, Desbois M, et al. Cancer-induced immunosuppression: IL-18-elicited immunoablative NK cells. Cancer Res 2012; 72(11):2757-67 doi 10.1158/0008-5472.CAN-11-3379.
22. Sun J C, Lanier L L. NK cell development, homeostasis and function: parallels with CD8(+) T cells. Nat Rev Immunol 2011; i1(10):645-57 doi 10.1038/nri3044.
23. Kondo M, Weissman I L, Akashi K. Identification of clonogenic common lymphoid progenitors in mouse bone marrow. Cell 1997; 91(5):661-72 doi 10.1016/s0092-8674(00)80453-5.
24. Min-Oo G, Kamimura Y, Hendricks D W, Nabekura T, Lanier L L. Natural killer cells: walking three paths down memory lane. Trends Immunol 2013; 34(6):251-8 doi 10.1016/j.it.2013.02.005.
25. Cooper M A, Elliott J M, Keyel P A, Yang L, Carrero J A, Yokoyama W M. Cytokine-induced memory-like natural killer cells. Proc Natl Acad Sci USA 2009; 106(6):1915-9 doi 10.1073/pnas.0813192106.
26. Ni J, Miller M, Stojanovic A, Garbi N, Cerwenka A. Sustained effector function of IL-12/15/18-preactivated NK cells against established tumors. J Exp Med 2012; 209(13):2351-65 doi 10.1084/jem.20120944.
27. Leong J W, Chase J M, Romee R, Schneider S E, Sullivan R P, Cooper M A, et al. Preactivation with IL-12, IL-15, and IL-18 induces CD25 and a functional high-affinity IL-2 receptor on human cytokine-induced memory-like natural killer cells. Biol Blood Marrow Transplant 2014; 20(4):463-73 doi 10.1016/j.bbmt.2014.01.006.
28. Romee R, Rosario M, Berrien-Elliott M M, Wagner J A, Jewell B A, Schappe T, et al. Cytokine-induced memory-like natural killer cells exhibit enhanced responses against myeloid leukemia. Sci Transl Med 2016; 8(357):357ra123 doi 10.1126/scitranslmed.aaf2341.
29. Liu E, Tong Y, Dotti G, Shaim H, Savoldo B, Mukherjee M, et al. Cord blood NK cells engineered to express IL-15 and a CD19-targeted CAR show long-term persistence and potent antitumor activity. Leukemia 2018; 32(2):520-31 doi 10.1038/leu.2017.226.
30. Berger C, Berger M, Hackman R C, Gough M, Elliott C, Jensen M C, et al. Safety and immunologic effects of IL-15 administration in nonhuman primates. Blood 2009; 114(12):2417-26 doi 10.1182/blood-2008-12-189266.
31. Waldmann T A, Lugli E, Roederer M, Perera L P, Smedley J V, Macallister R P, et al. Safety (toxicity), pharmacokinetics, immunogenicity, and impact on elements of the normal immune system of recombinant human IL-15 in rhesus macaques. Blood 2011; 117(18):4787-95 doi 10.1182/blood-2010-10-311456.
32. Perera P Y, Lichy J H, Waldmann T A, Perera L P. The role of interleukin-15 in inflammation and immune responses to infection: implications for its therapeutic use. Microbes Infect 2012; 14(3):247-61 doi 10.1016/j.micinf.2011.10.006.
33. Guo Y, Luan L, Rabacal W, Bohannon J K, Fensterheim B A, Hernandez A, et al. IL-15 Superagonist-Mediated Immunotoxicity: Role of NK Cells and IFN-gamma. J Immunol 2015; 195(5):2353-64 doi 10.4049/jimmunol.1500300.
34. Conlon K C, Lugli E, Welles H C, Rosenberg S A, Fojo A T, Morris J C, et al. Redistribution, hyperproliferation, activation of natural killer cells and CD8 T cells, and cytokine production during first-in-human clinical trial of recombinant human interleukin-15 in patients with cancer. J Clin Oncol 2015; 33(1):74-82 doi 10.1200/JCO.2014.57.3329.
35. Conlon K C, Potter E L, Pittaluga S, Lee C R, Miljkovic M D, Fleisher T A, et al. IL15 by Continuous Intravenous Infusion to Adult Patients with Solid Tumors in a Phase I Trial Induced Dramatic NK-Cell Subset Expansion. C/in Cancer Res 2019; 25(16):4945-54 doi 10.1158/1078-0432.CCR-18-3468.
36. Christodoulou I, Ho W J, Marple A, Ravich J W, Tam A, Rahnama R, et al. Engineering CAR-NK cells to secrete IL-15 sustains their anti-AML functionality but is associated with systemic toxicities. J Immunother Cancer 2021; 9(12) doi 10.1136/jitc-2021-003894.
37. Biederstadt A, Rezvani K. Engineering the next generation of CAR-NK immunotherapies. Int J Hematol 2021; 114(5):554-71 doi 10.1007/s12185-021-03209-4.
38. Tang H, Liang Y, Anders R A, Taube J M, Qiu X, Mulgaonkar A, et al. PD-L1 on host cells is essential for PD-L1 blockade-mediated tumor regression. J Cin Invest 2018; 128(2):580-8 doi 10.1172/JC196061.

INFORMAL SEQUENCE LISTING
Nucleic acid encoding IL-2 signal peptide
SEQ ID NO: 1
ATGTACAGGATGCAACTCCTGTCTTGCATTGCACTAAGTCTTGCA
CTTGTCACAAACAGT
Nucleic acid encoding IL-15 protein
SEQ ID NO: 1
GGCATTCATGTCTTCATTTTGGGCTGTTTCAGTGCAGGGCTTCCT
AAAACAGAAGCCAACTGGGTGAATGTAATCAGCGACCTCAAGAAG
ATCGAGGACTTGATCCAGTCCATGCACATAGATGCTACTTTATAT
ACGGAAAGTGATGTTCACCCCAGTTGCAAAGTAACAGCAATGAAG
TGCTTTCTCTTGGAGTTACAAGTTATTTCACTTGAGTCCGGAGAT
GCAAGTATTCATGATACAGTAGAAAATCTGATCATCCTAGCAAAC
AACAGTTTGTCTTCTAATGGGAATGTAACAGAATCTGGATGCAAA
GAATGTGAGGAACTGGAGGAAAAAAATATTAAAGAATTTTTGCAG
AGTTTTGTACATATTGTCCAAATGTTCATCAACACTTCT
Nucleic acid encoding soluble IL-15
SEQ ID NO: 3
ATGTACAGGATGCAACTCCTGTCTTGCATTGCACTAAGTCTTGCA
CTTGTCACAAACAGTGGCATTCATGTCTTCATTTTGGGCTGTTTC
AGTGCAGGGCTTCCTAAAACAGAAGCCAACTGGGTGAATGTAATC
AGCGACCTCAAGAAGATCGAGGACTTGATCCAGTCCATGCACATA
GATGCTACTTTATATACGGAAAGTGATGTTCACCCCAGTTGCAAA
GTAACAGCAATGAAGTGCTTTCTCTTGGAGTTACAAGTTATTTCA
CTTGAGTCCGGAGATGCAAGTATTCATGATACAGTAGAAAATCTG
ATCATCCTAGCAAACAACAGTTTGTCTTCTAATGGGAATGTAACA
GAATCTGGATGCAAAGAATGTGAGGAACTGGAGGAAAAAAATATT
AAAGAATTTTTGCAGAGTTTTGTACATATTGTCCAAATGTTCATC
AACACTTCT
IL-2 signal peptide
SEQ ID NO: 4
MYRMQLLSCIALSLALVTNS
IL-15 protein
SEQ ID NO: 5
GIHVFILGCFSAGLPKTEANWVNVISDLKKIEDLIQSMHIDATLY
TESDVHPSCKVTAMKCFLLELQVISLESGDASIHDTVENLIILAN
NSLSSNGNVTESGCKECEELEEKNIKEFLQSFVHIVQMFINTS
soluble IL-15
SEQ ID NO: 6
MYRMQLLSCIALSLALVTNSGIHVFILGCFSAGLPKTEANWVNVI
SDLKKIEDLIQSMHIDATLYTESDVHPSCKVTAMKCFLLELQVIS
LESGDASIHDTVENLIILANNSLSSNGNVTESGCKECEELEEKNI
KEFLQSFVHIVQMFINTS

1. A PD-L1(+) natural killer cell, wherein the PD-L1(+) natural killer cell expresses soluble IL-15.

2. The PD-L1(+) natural killer cell of claim 1, wherein the PD-L1(+) natural killer cell expresses soluble IL-15 and truncated EGFR.

3-77. (canceled)

78. The PD-L1(+) natural killer cell of claim 1, wherein:
(a) the PD-L1(+) natural killer cell is an activated cord blood natural killer cell;
(b) the PD-L1(+) natural killer cell was derived from an umbilical cord blood natural killer cell; or
(c) the PD-L1(+) natural killer cell does not express a CD19 chimeric antigen receptor.

79. A population of PD-L1(+) natural killer cells of claim 1.

80. A pharmaceutical composition comprising the PD-L1(+) natural killer cell of claim 1.

81. A method of treating cancer in a patient in need thereof, the method comprising administering to the patient an effective amount the PD-L1(+) natural killer cell of claim 1.

82. The method of claim 81, wherein the effective amount is from about 1×10⁶ to about 1×10¹² of PD-L1(+) natural killer cells.

83. The method of claim 81, wherein the effective amount is from about 1×10⁷ to about 1×10¹² of the PD-L1(+) natural killer cells; from about 2×10⁷ to about 1×10¹⁰ of the PD-L1(+) natural killer cells; from about 4×10⁷ to about 2×10⁹ of the PD-L1(+) natural killer cells; from about 1×10⁸ to about 2×10⁹ of the PD-L1(+) natural killer cells; about 5×10⁷ of the PD-L1(+) natural killer cells; about 1×10⁸ of the PD-L1(+) natural killer cells; about 2×10⁸ of the PD-L1(+) natural killer cells; about 4×10⁸ of the PD-L1(+) natural killer cells; about 5×10⁸ of the PD-L1(+) natural killer cells; or about 1×10⁹ of the PD-L1(+) natural killer cells.

84. The method of claim 81, comprising administering the PD-L1(+) natural killer cells to the patient about twice per week; about once per day; about once every two days; about once every three days; about once every four days; about once every five days; about once every six days; about once every three or four days; about once per week; once per week for four weeks; once per week for five weeks; once per week for six weeks; once per week for seven weeks; once per week for eight weeks; twice per week for four weeks; twice per week for five weeks; twice per week for six weeks; twice per week for seven weeks; or twice per week for eight weeks.

85. The method of claim 81, wherein:
(a) the effective amount of PD-L1(+) natural killer cells are administered to the patient once per week for four weeks; wherein administration of the PD-L1(+) natural killer cells is discontinued for 4 weeks; and wherein another 8 week treatment cycle is optionally initiated one or more times thereafter;
(b) the effective amount of PD-L1(+) natural killer cells are administered to the patient once per week for five weeks; wherein administration of the PD-L1(+) natural killer cells is discontinued for 3 weeks; and wherein another 8 week treatment cycle is optionally initiated one or more times thereafter;
(c) the effective amount of PD-L1(+) natural killer cells are administered to the patient once per week for six weeks; wherein administration of the PD-L1(+) natural killer cells is discontinued for 2 weeks; and wherein another 8 week treatment cycle is optionally initiated one or more times thereafter;
(d) the effective amount of PD-L1(+) natural killer cells are administered to the patient once per week for seven weeks; wherein administration of the PD-L1(+) natural killer cells is discontinued for 1 week; and wherein another 8 week treatment cycle is optionally initiated one or more times thereafter;
(e) the effective amount of PD-L1(+) natural killer cells are administered to the patient twice per week for four weeks; wherein administration of the PD-L1(+) natural killer cells is discontinued for 4 weeks; and wherein another 8 week treatment cycle is optionally initiated one or more times thereafter;
(f) the effective amount of PD-L1(+) natural killer cells are administered to the patient twice per week for five weeks; wherein administration of the PD-L1(+) natural killer cells is discontinued for 3 weeks; and wherein another 8 week treatment cycle is optionally initiated one or more times thereafter;
(g) the effective amount of PD-L1(+) natural killer cells are administered to the patient twice per week for six weeks; wherein administration of the PD-L1(+) natural killer cells is discontinued for 2 weeks; and wherein another 8 week treatment cycle is optionally initiated one or more times thereafter; or
(h) the effective amount of PD-L1(+) natural killer cells are administered to the patient twice per week for seven weeks; wherein administration of the PD-L1(+) natural killer cells is discontinued for 1 week; and wherein another 8 week treatment cycle is optionally initiated one or more times thereafter.

86. The method of claim 81, wherein the PD-L1(+) natural killer cells are administered intravenously as an infusion for about 30 minutes to about 120 minutes.

87. The method of claim 81, further comprising pretreating the patient with a low-dose or an escalating dose of the PD-L1(+) natural killer cells prior to beginning treatment with the effective dose, wherein the low-dose or escalating dose is an amount lower than the effective dose.

88. The method of claim 81, further comprising administering to the patient an effective amount of a checkpoint inhibitor

89. The method of claim 88, wherein the checkpoint inhibitor is a PD-1 inhibitor or a PD-L1 inhibitor.

90. The method of claim 81, wherein the cancer is non-small cell lung cancer.

91. The method of claim 81, wherein the cancer is lung cancer, neuroblastoma, glioma, myelodysplastic syndrome, B cell lymphoma, chronic myeloid leukemia, acute myeloid leukemia, liver cancer, prostate cancer, pancreatic cancer, gastric cancer, head and neck cancer, multiple myeloma, biliary tract cancer, ovarian cancer, lymphoblastic leukemia, melanoma, non-Hodgkin lymphoma, or colorectal cancer.

92. A nucleic acid encoding a soluble IL-15 protein, wherein the soluble IL-15 protein comprises an IL-2 signal peptide and an IL-15 protein.

93. The nucleic acid of claim 92, wherein the nucleic acid comprises the nucleotide sequence of SEQ ID NO:1, SEQ ID NO:2, or SEQ ID NO:3.

94. A recombinant soluble IL-15 protein, wherein the soluble IL-15 protein comprises an IL-2 signal peptide and an IL-15 protein.

95. The recombinant soluble IL-15 protein of claim 94, wherein the soluble IL-15 protein comprises the amino acid sequence of SEQ ID NO:4, SEQ ID NO:5, or SEQ ID NO:6.


SEQUENCE LISTING (6 sequences)

SEQ ID NO: 1 - DNA, 60 bp, synthetic construct
  source 1..60: mol_type = other DNA
atgtacaggatgcaactcctgtcttgcattgcactaagtcttgcacttgtcacaaacagt

SEQ ID NO: 2 - DNA, 399 bp, synthetic construct
  source 1..399: mol_type = other DNA
ggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 3 - DNA, 459 bp, synthetic construct
  source 1..459: mol_type = other DNA
atgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 4 - protein, 20 aa, synthetic construct
MYRMQLLSCIALSLALVTNS

SEQ ID NO: 5 - protein, 133 aa, synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 153 aa, synthetic construct
MYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS